(19) United States
(12) Patent Application Publication
(10) Pub. No.: US 2025/0320294 A1
(43) Pub. Date: Oct. 16, 2025

(54) BISPECIFIC ANTIBODY FOR T-CELL MODULATION

(71) Applicant: The Trustees of Columbia University in the City of New York, New York, NY (US)

(72) Inventors: Adam MOR, Old Tappan, NJ (US); Yevgeniya GARTSHTEYN, Glen Rock, NJ (US); Ruijiang SONG, Forest Hills, NY (US); Shalom LERRER, Tenafly, NJ (US)

(21) Appl. No.: 19/080,584

(22) Filed: Mar. 14, 2025

Related U.S. Application Data
(63) Continuation-in-part of application No. PCT/US23/74393, filed on Sep. 15, 2023.
(60) Provisional application No. 63/375,980, filed on Sep. 16, 2022.
(60) Provisional application No. 63/696,208, filed on Sep. 18, 2024.

(51) Int. Cl. C07K 16/28

(52) U.S. Cl. CPC C07K 16/2803; C07K 16/2809; C07K 16/289; C07K 16/2896; C07K 2317/31; C07K 2317/92; C07K 2319/00

(57) ABSTRACT

The subject matter described here relates to bispecific antibodies capable of modulating T-cell activity, wherein the two arms of the bispecific antibody are against CD3 and SLAMF6, against CD45 and SLAMF6, or against CD43 and SLAMF6.

This application is a continuation-in-part of PCT/US2023/074393, filed Sep. 15, 2023, which claims the benefit of and priority to U.S. Provisional Patent Application No. 63/375,980, filed Sep. 16, 2022, the contents of each of which are hereby incorporated by reference in their entireties. This application also claims the benefit of and priority to U.S. Provisional Patent Application No. 63/696,208, filed Sep. 18, 2024, the contents of which is hereby incorporated by reference in its entirety.

This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### GOVERNMENT SUPPORT

This invention was made with government support under grant numbers AI125640, and AI150597 awarded by the National Institutes of Health. The government has certain rights in the invention.

### INCORPORATION BY REFERENCE

All documents cited herein are incorporated herein by reference in their entireties.

### SEQUENCE LISTING

The instant application contains a Sequence Listing which has been submitted electronically in XML format and is hereby incorporated by reference in its entirety. Said XML copy, created on Mar. 10, 2025, is named 0019240_01287US3_SL.xml and is 41,880 bytes in size.

### TECHNICAL FIELD

The present invention relates generally to antibodies and expression systems for producing antibodies. More particularly, the present invention relates to anti-CD3×SLAMF6, anti-CD45×SLAMF6, and anti-CD43×SLAMF6 bispecific antibodies.

### BACKGROUND

Activation of T-cells occurs when a cognate peptide engages the CD3 T-cell receptor (TCR), the latter presented on antigen-presenting cells (APCs). This interaction occurs at the immunologic synapse (IS) and is fine-tuned by co-receptors that co-localize with the CD3 in the contact zones of the IS. Co-receptor signals may be activating, as in the case of CD28, or inhibitory as in the case of PD-1 and CTLA-4. Furthermore, the recruitment or exclusion of the co-receptors to and from the IS affects how these receptors modulate T-cell functions.

Signaling lymphocyte activation molecule (SLAMF6) (also referred to as Ly108 in mice or NTB-A in humans) is a cell surface receptor expressed on a wide variety of hematopoietic cells. The homophilic interaction of SLAMF6 in trans between T-cells and APCs promotes a stable cell-cell interaction. Activation of the SLAMF6 co-receptor on T-cells results in the recruitment of SRC family kinases and subsequent phosphorylation of cytoplasmic immunoreceptor tyrosine-based switch motifs (ITSM). These secondary signals culminate in the immunomodulation of the TCR response.

The present disclosure is directed at bioengineered SLAMF6 bispecific antibodies (anti-CD3/SLAMF6, anti-CD45/SLAMF6, and anti-CD43/SLAMF6) that can modulate T-cell responses to provide a novel therapeutic target of T-cell activity in disease. The bispecific antibodies regulate T-cell activity by targeting SLAMF6 on the cell membrane.

### SUMMARY OF THE INVENTION

In certain aspects, described herein is a bispecific antibody or a fragment thereof, comprising: a first arm comprising a first variable heavy chain domain and a first variable light chain domain, wherein a portion of the first arm is capable of binding to a portion of a CD3 protein, a portion of a CD45, portion, or a portion of a CD43 protein; and a second arm comprising a second variable heavy chain domain and a second variable light chain domain, wherein a portion of the second arm is capable of binding to a portion of a SLAMF6 protein.

In some embodiments, the first arm is encoded by a first polypeptide chain and the second arm is encoded by a second polypeptide chain that associate together. In some embodiments, the first arm comprises a linker between the first variable heavy domain and first variable light chain domain. In some embodiments, the second arm comprises a linker between the first variable heavy domain and first variable light chain domain. In some embodiments, the linker is a glycine-serine linker.

In some embodiments, the first and second arms each further comprise a fragment, crystallizable (Fc) region. In some embodiments, the Fc region of the first arm comprises knob mutations and the Fc region of the second arm comprise hole mutations, or vice versa.

In some embodiments, the bispecific antibody is bivalent. In some embodiments, the first arm and second arm are encoded on a first polypeptide chain.

In some embodiments, the first polypeptide chain further comprises: a third arm comprising a third variable heavy chain domain and a third variable light chain domain that is the same as the first variable heavy chain domain and first variable light chain domain; and the second polypeptide chain further comprises: a fourth arm comprising a fourth variable heavy chain domain and a fourth variable light chain domain, that is the same as the second variable heavy chain domain and second variable light chain domain.

In some embodiments, the first arm comprises a linker between the first variable heavy domain and first variable light chain domain. In some embodiments, the second arm comprises a linker between the first variable heavy domain and first variable light chain domain. In some embodiments, the third arm comprises a linker between the third variable heavy domain and third variable light chain domain. In some embodiments, the fourth arm comprises a linker between the fourth variable heavy domain and fourth variable light chain domain. In some embodiments, the linker is a glycine-serine linker.

In some embodiments, the first and second polypeptide chains each further comprises a fragment, crystallizable (Fc) region. In some embodiments, the Fc region of the first polypeptide chain comprises knob mutations and the Fc region of the second polypeptide chain comprises hole mutations, or vice versa. In some embodiments, the Fc region of the first polypeptide chain is positioned between the first arm and third arm and the Fc region of the second polypeptide chain is positioned between the second arm and fourth arm. In some embodiments, the bispecific antibody is tetravalent.

In some embodiments, the first variable heavy chain domain comprises an amino acid sequence of the variable heavy chain portion of SEQ ID NO: 2, wherein the first variable light chain domain comprises an amino acid sequence of the variable light chain portion of SEQ ID NO: 2. In some embodiments, the first arm comprises SEQ ID NO: 2, wherein the second arm comprises SEQ ID NO: 9. In some embodiments, the first variable heavy chain domain comprises an amino acid sequence of the variable heavy chain portion of SEQ ID NO: 5, wherein the first variable light chain domain comprises an amino acid sequence of the variable light chain portion of SEQ ID NO: 5. In some embodiments, the first arm comprises SEQ ID NO: 5, wherein the second arm comprises SEQ ID NO: 9. In some embodiments, the first variable heavy chain domain comprises an amino acid sequence of the variable heavy chain portion of SEQ ID NO: 28, wherein the first variable light chain domain comprises an amino acid sequence of the variable light chain portion of SEQ ID NO: 28. In some embodiments, the first arm comprises SEQ ID NO: 28, wherein the second arm comprises SEQ ID NO: 9. In some embodiments, the first variable heavy chain domain comprises an amino acid sequence of the variable heavy chain portion of SEQ ID NO: 29, wherein the first variable light chain domain comprises an amino acid sequence of the variable light chain portion of SEQ ID NO: 29. In some embodiments, the first arm comprises SEQ ID NO: 29, wherein the second arm comprises SEQ ID NO: 9. In some embodiments, the first variable heavy chain domain comprises an amino acid sequence of the variable heavy chain portion of SEQ ID NO: 30, wherein the first variable light chain domain comprises an amino acid sequence of the variable light chain portion of SEQ ID NO: 30. In some embodiments, the first arm comprises SEQ ID NO: 30, wherein the second arm comprises SEQ ID NO: 9. In some embodiments, the first variable heavy chain domain comprises an amino acid sequence of the variable heavy chain portion of SEQ ID NO: 31, wherein the first variable light chain domain comprises an amino acid sequence of the variable light chain portion of SEQ ID NO: 31. In some embodiments, the first arm comprises SEQ ID NO: 31, wherein the second arm comprises SEQ ID NO: 9. In some embodiments, the first variable heavy chain domain comprises an amino acid sequence of the variable heavy chain portion of SEQ ID NO: 32, wherein the first variable light chain domain comprises an amino acid sequence of the variable light chain portion of SEQ ID NO: 32. In some embodiments, the first arm comprises SEQ ID NO: 32, wherein the second arm comprises SEQ ID NO: 9. In some embodiments, the first variable heavy chain domain comprises an amino acid sequence of the variable heavy chain portion of SEQ ID NO: 33, wherein the first variable light chain domain comprises an amino acid sequence of the variable light chain portion of SEQ ID NO: 33. In some embodiments, the first arm comprises SEQ ID NO: 33, wherein the second arm comprises SEQ ID NO: 9.

In some embodiments, the first variable heavy chain domain comprises an amino acid sequence of the variable heavy chain portion of SEQ ID NO: 7, wherein the first variable light chain domain comprises an amino acid sequence of the variable light chain portion of SEQ ID NO: 7. In some embodiments, the first arm comprises SEQ ID NO: 7, wherein the second arm comprises SEQ ID NO: 9.

In some embodiments, the second variable heavy chain domain comprises an amino acid sequence of the variable heavy chain portion of SEQ ID NO: 9, wherein the first variable light chain domain comprises an amino acid sequence of the variable light chain portion of SEQ ID NO: 9. In some embodiments, the first arm comprises an amino acid sequence selected from the group consisting of SEQ ID NO: 2, SEQ ID NO: 5, SEQ ID NO: 7, SEQ ID NO: 28, SEQ ID NO: 29, SEQ ID NO: 30, SEQ ID NO: 31, SEQ ID NO: 32, or SEQ ID NO: 33 and wherein the second arm comprises SEQ ID NO: 9.

In some embodiments, the portion of the first arm is capable of binding to the portion of the CD3 protein, and wherein the bispecific antibody is capable of clustering a SLAMF6 protein with a core of an immune synapse. In some embodiments, the bispecific antibody is capable of promoting downstream signaling of a SLAMF6 mediated response in a T cell. In some embodiments, the bispecific antibody is capable of enhancing T cell function. In some embodiments, the bispecific antibody enhancement of T cell function is greater than either monospecific antibody does individually or in a nonspecific combination mixture. In some embodiments, the bispecific antibody enhancement of T cell function is dose-dependent. In some embodiments, the bispecific antibody is capable of binding to the portion of the CD3 protein and to the portion of the SLAMF6 protein, wherein the CD3 protein and SLAMF6 protein are located on a same T cell.

In some embodiments, the portion of the first arm is capable of binding to the portion of the CD45 protein, and wherein the bispecific antibody is capable of localizing a SLAMF6 protein away from a core of an immune synapse.

In some embodiments, the bispecific antibody is capable of disrupting downstream signaling of inhibitory proteins and effector pathways in a T cell. In some embodiments, the bispecific antibody is capable of enhancing T cell function. In some embodiments, the bispecific antibody enhancement of T cell function is greater than either monospecific antibody does individually or in a nonspecific combination mixture. In some embodiments, the bispecific antibody enhancement of T cell function is dose-dependent.

In some embodiments, the bispecific antibody is capable of binding to the portion of the CD45 protein and to the portion of the SLAMF6 protein, wherein the CD45 protein and SLAMF6 protein are located on a same T cell. In some embodiments, the portion of the first arm is capable of binding to the portion of the CD43 protein, and wherein the bispecific antibody is capable of localizing a SLAMF6 protein away from a core of an immune synapse. In some embodiments, the bispecific antibody is capable of disrupting a downstream signaling of inhibitory proteins and effector pathways in a T cell. In some embodiments, the bispecific antibody is capable of enhancing T cell function. In some embodiments, the bispecific antibody enhancement of T cell function is greater than either monospecific antibody does individually or in a nonspecific combination mixture. In some embodiments, the bispecific antibody enhancement of T cell function is dose-dependent. In some embodiments, the bispecific antibody is capable of binding to the portion of the CD43 protein and to the portion of the SLAMF6 protein, wherein the CD43 protein and SLAMF6 protein are located on a same T cell.

In some embodiments, the SLAMF6 protein is located on a T cell, and wherein the bispecific antibody is capable of promoting binding of a SLAM associated protein (SAP) to an intracellular tail of the SLAMF6 protein on a tumor cell. In some embodiments, the bispecific antibody is capable of inducing a cytokine secretion in a T cell. In some embodiments, the cytokine secretion is a secretion of IL-2.

In certain aspects, described herein is a tetravalent bispecific antibody or a fragment thereof, comprising: (I) a first fragment antigen-binding (Fab) region, comprising: a first arm comprising a first variable heavy chain domain and a first variable light chain domain, wherein a portion of the first arm is capable of binding to a portion of a CD3 protein, a portion of a CD45 protein, or a portion of a CD43 protein; and a second arm comprising a second variable heavy chain domain and a second variable light chain domain, wherein a portion of the second arm is capable of binding to a portion of a CD3 protein, a portion of a CD45 protein, or a portion of a CD43 protein; (II) a first fragment, crystallizable (Fc) region; (III) a second Fab region, comprising: a third arm comprising a first variable heavy chain domain and a first variable light chain domain, wherein a portion of the first arm is capable of binding to a portion of a SLAMF6 protein; and a fourth arm comprising a second variable heavy chain domain and a second variable light chain domain, wherein a portion of the second arm is capable of binding to a portion of a SLAMF6 protein; and (IV) a second Fc region, wherein the first and second Fc regions associated together.

In some embodiments, one arm of the first Fab region is N-terminal to the first Fc region and the other arm of the first Fab region is C-terminal to the first Fc region, and the first arm of the second Fab region is N-terminal to the second Fc region and the other arm of the second Fab region is C-terminal to the second Fc region. In some embodiments, one arm of the first Fab region is C-terminal to the first Fc region and one arm of the second Fab region is N-terminal to the first Fc region, and the other arm of the first Fab region is C-terminal to the second Fc region and the other arm of the second Fab region is N-terminal to the second Fc region, or one arm of the first Fab region is N-terminal to the first Fc region and one arm of the second Fab region is C-terminal to the first Fc region, and the other arm of the first Fab region is N-terminal to the second Fc region and the other arm of the second Fab region is C-terminal to the second Fc region.

In certain aspects, described herein is a pharmaceutical composition comprising: the bispecific antibody or the tetravalent bispecific antibody of any of the embodiments as described above herein; and a pharmaceutically acceptable carrier.

In certain aspects, described herein is a method of preventing or treating cancer in a subject comprising administering to the subject an effective amount of the pharmaceutical composition as described above herein. In some embodiments, the cancer is selected from colorectal cancer, lung cancer, bladder cancer, breast cancer, cervical cancer, kidney cancer, leukemia, Hodgkin lymphoma, non-Hodgkin lymphoma, prostate cancer, skin cancer (e.g., melanoma), head and neck cancer, endometrial cancer, colon cancer, rectal cancer, liver cancer, thyroids cancer, esophageal cancer, renal cell cancer, and a combination thereof.

In certain aspects, described above herein is a method of preventing or treating an autoimmune disease in a subject comprising administering to the subject an effective amount of the pharmaceutical composition as described above herein. In some embodiments, the autoimmune disease is Systemic lupus erythematosus (Lupus).

In some embodiments, the enhancement of T-cell activation is indicated by IFN-γ levels. In some embodiments, the enhancement of T-cell activation is indicated by phosphorylation of CD3 zeta.

In certain aspects, described above herein is a kit for generating a bispecific antibody or fragment thereof, the kit comprising one or more vectors comprising a polynucleotide sequence encoding any of the bispecific antibodies as described above herein.

In certain aspects, described above herein is a kit for generating a tetravalent bispecific antibody or fragment thereof, the kit comprising one or more vectors comprising a polynucleotide sequence encoding any of the tetravalent bispecific antibodies as described above herein.

In certain aspects, described above herein is a kit for generating a bispecific antibody or fragment thereof, the kit comprising: a first vector comprising a polynucleotide sequence encoding a first arm of the bispecific antibody or fragment thereof, wherein a portion of the first arm is capable of binding to a portion of a CD3, protein, a portion of a CD45 protein, or a portion of a CD43 protein; and a second vector comprising a polynucleotide sequence encoding a second arm of the bispecific antibody or fragment thereof, wherein a portion of the second arm is capable of binding to a portion of a SLAMF6 protein.

In some embodiments, the first vector and the second vector are the same vector. In some embodiments, the first vector and the second vector are two different vectors. In some embodiments, a variable heavy chain domain of the first arm comprises an amino acid sequence of the variable heavy chain portion of SEQ ID NO: 2, 5, 7, 28, 29, 30, 31, 32, or 33 a first variable light chain domain of the first arm comprises an amino acid sequence of the variable light chain portion of SEQ ID NO: 2, 5, 7, 28, 29, 30, 31, 32, or 33 a variable heavy chain domain of the second arm comprises an amino acid sequence of the variable heavy chain portion of SEQ ID NO: 9, and a variable light chain domain of the second arm comprises an amino acid sequence of the variable light chain portion of SEQ ID NO: 9.

In some embodiments, the first arm comprises an amino acid sequence selected from the group consisting of SEQ ID NO: 2, SEQ ID NO: 5, SEQ ID NO: 7, SEQ ID NO: 28, SEQ ID NO: 29, SEQ ID NO: 30, SEQ ID NO: 31, SEQ ID NO: 32, and SEQ ID NO: 33 wherein the second arm comprises SEQ ID NO: 9. In some embodiments, the first arm comprises SEQ ID NO: 2. In some embodiments, the first arm comprises SEQ ID NO: 5. In some embodiments, the first arm comprises SEQ ID NO: 7. In some embodiments, the first arm comprises SEQ ID NO: 28. In some embodiments, the first arm comprises SEQ ID NO: 29. In some embodiments, the first arm comprises SEQ ID NO: 30. In some embodiments, the first arm comprises SEQ ID NO: 31. In some embodiments, the first arm comprises SEQ ID NO: 32. In some embodiments, the first arm comprises SEQ ID NO: 33.

In some embodiments, the kit for generating a tetravalent bispecific antibody or fragment thereof comprises one or more vectors comprising a polynucleotide sequence encoding an amino acid sequence of SEQ ID NO: 11, SEQ ID NO: 12, SEQ ID NO: 13, SEQ ID NO: 22, SEQ ID NO: 23, SEQ ID NO: 24, SEQ ID NO: 25, SEQ ID NO: 26, or SEQ ID NO: 27 and a polynucleotide sequence encoding an amino acid sequence of SEQ ID NO: 16.

In some embodiments, disclosed herein is one or more host cells comprising one or more vectors comprising a polynucleotide sequence encoding an amino acid sequence of SEQ ID NO: 11, SEQ ID NO: 12, SEQ ID NO: 13, SEQ ID NO: 22, SEQ ID NO: 23, SEQ ID NO: 24, SEQ ID NO: 25, SEQ ID NO: 26, or SEQ ID NO: 27 and a polynucleotide sequence encoding an amino acid sequence of SEQ ID NO: 16.

In some embodiments, disclosed herein is a composition comprising one or more vectors comprising a polynucleotide sequence encoding an amino acid sequence of SEQ ID NO: 11, SEQ ID NO: 12, SEQ ID NO: 13, SEQ ID NO: 22, SEQ ID NO: 23, SEQ ID NO: 24, SEQ ID NO: 25, SEQ ID NO: 26, or SEQ ID NO: 27 and a polynucleotide sequence encoding an amino acid sequence of SEQ ID NO: 16.

In certain aspects, described above herein is one or more host cells comprising: one or more vectors comprising a polynucleotide sequence encoding any of the bispecific antibodies or any of the tetravalent bispecific antibodies as described above herein.

In certain aspects, described above herein is one or more host cells comprising: a first vector comprising a polynucleotide sequence encoding a first arm of a bispecific antibody or fragment thereof, wherein a portion of the first arm is capable of binding to a portion of a CD3 protein, a portion of a CD45 protein, or a portion of a CD43 protein; and a second vector comprising a polynucleotide sequence encoding a second arm of the bispecific antibody or fragment thereof, wherein a portion of the second arm is capable of binding to a portion of a SLAMF6 protein.

In some embodiments, the first vector and the second vector are the same vector. In some embodiments, the first vector and the second vector are two different vectors. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 2, and the first variable light chain domain comprises an amino acid sequence of the variable light chain portion of SEQ ID NO: 2. In some embodiments, the first arm comprises SEQ ID NO: 2, wherein the second arm comprises SEQ ID NO: 9.

In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 5, and the first variable light chain domain comprises an amino acid sequence of the variable light chain portion of SEQ ID NO: 5. In some embodiments, the first arm comprises SEQ ID NO: 5, and the second arm comprises SEQ ID NO: 9.

In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 7, and the first variable light chain domain comprises an amino acid sequence of the variable light chain portion of SEQ ID NO: 7. In some embodiments, the first arm comprises SEQ ID NO: 7, and the second arm comprises SEQ ID NO: 9.

In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 28, and the first variable light chain domain comprises an amino acid sequence of the variable light chain portion of SEQ ID NO: 28. In some embodiments, the first arm comprises SEQ ID NO: 28, and the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 29, and the first variable light chain domain comprises an amino acid sequence of the variable light chain portion of SEQ ID NO: 29. In some embodiments, the first arm comprises SEQ ID NO: 29, and the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 30, and the first variable light chain domain comprises an amino acid sequence of the variable light chain portion of SEQ ID NO: 30. In some embodiments, the first arm comprises SEQ ID NO: 30, and the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 31, and the first variable light chain domain comprises an amino acid sequence of the variable light chain portion of SEQ ID NO: 31. In some embodiments, the first arm comprises SEQ ID NO: 31, and the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 32, and the first variable light chain domain comprises an amino acid sequence of the variable light chain portion of SEQ ID NO: 32. In some embodiments, the first arm comprises SEQ ID NO: 32, and the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 33, and the first variable light chain domain comprises an amino acid sequence of the variable light chain portion of SEQ ID NO: 33. In some embodiments, the first arm comprises SEQ ID NO: 10, and the second arm comprises SEQ ID NO: 9.

In certain aspects, described above herein is a method of making a bispecific antibody or fragment thereof comprising: culturing the one or more host cells as described above herein under conditions suitable for an expression of the one or more vectors; and recovering the bispecific antibody or fragment thereof or tetravalent bispecific antibody or fragment thereof.

In certain aspects, described above herein is a method of making a bispecific antibody or fragment thereof comprising: culturing the one or more host cells as described above herein under conditions suitable for an expression of the first vector and the second vector; and recovering the bispecific antibody or fragment thereof.

In certain aspects, described above herein is a composition comprising: one or more vectors comprising a polynucleotide sequence encoding any of the bispecific antibodies or any of the tetravalent bispecific antibodies as described above herein.

In certain aspects, described above herein is a composition comprising: a first vector comprising a polynucleotide sequence encoding a first arm of the bispecific antibody or fragment thereof, wherein a portion of the first arm is capable of binding to a portion of a CD3 protein, a portion of a CD45 protein, or a portion of a CD43 protein; and a second vector comprising a polynucleotide sequence encoding a second arm of the bispecific antibody or fragment thereof, wherein a portion of the second arm is capable of binding to a portion of a SLAMF6 protein.

In some embodiments, the first vector and the second vector are the same vector. In some embodiments, the first vector and the second vector are two different vectors. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 2, and the first variable light chain domain comprises an amino acid sequence of the variable light chain portion of SEQ ID NO: 2. In some embodiments, the first arm comprises SEQ ID NO: 2, and the second arm comprises SEQ ID NO: 9.

In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 5, and the first variable light chain domain comprises an amino acid sequence of the variable light chain portion of SEQ ID NO: 5. In some embodiments, the first arm comprises SEQ ID NO: 5, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 7, and the first variable light chain domain comprises an amino acid sequence of the variable light chain portion of SEQ ID NO: 7. In some embodiments, the first arm comprises SEQ ID NO: 7, and the second arm comprises SEQ ID NO: 9.

In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 28, and the first variable light chain domain comprises an amino acid sequence of the variable light chain portion of SEQ ID NO: 28. In some embodiments, the first arm comprises SEQ ID NO: 28, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 29, and the first variable light chain domain comprises an amino acid sequence of the variable light chain portion of SEQ ID NO: 29. In some embodiments, the first arm comprises SEQ ID NO: 29, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 30, and the first variable light chain domain comprises an amino acid sequence of the variable light chain portion of SEQ ID NO: 30. In some embodiments, the first arm comprises SEQ ID NO: 30, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 31, and the first variable light chain domain comprises an amino acid sequence of the variable light chain portion of SEQ ID NO: 31. In some embodiments, the first arm comprises SEQ ID NO: 31, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 32, and the first variable light chain domain comprises an amino acid sequence of the variable light chain portion of SEQ ID NO: 32. In some embodiments, the first arm comprises SEQ ID NO: 32, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 33, and the first variable light chain domain comprises an amino acid sequence of the variable light chain portion of SEQ ID NO: 33. In some embodiments, the first arm comprises SEQ ID NO: 33, wherein the second arm comprises SEQ ID NO: 9.

In certain aspects, described above herein is a means for binding: a portion of a CD3 protein, a portion of a CD45 protein, or a portion of a CD43 protein; and a portion of a SLAMF6 protein. In some embodiments, the means comprises a bispecific antibody or fragment thereof.

In some embodiments, the bispecific antibody or a fragment thereof comprises: a first arm comprising a first variable heavy chain domain and a first variable light chain domain, wherein a portion of the first arm is capable of binding to the portion of the CD3 protein, the portion of the CD45 protein, or the portion of the CD43 protein; and a second arm comprising a second variable heavy chain domain and a second variable light chain domain, wherein a portion of the second arm is capable of binding to the portion of the SLAMF6 protein. In some embodiments, the first variable heavy chain domain comprises an amino acid sequence of the variable heavy chain portion of SEQ ID NO: 2, the first variable light chain domain comprises an amino acid sequence of the variable light chain portion of SEQ ID NO: 2, and the portion of the first arm is capable of binding to the portion of the CD3 protein. In some embodiments, the first arm comprises SEQ ID NO: 2, the second arm comprises SEQ ID NO: 9, and the portion of the first arm is capable of binding to the portion of the CD3 protein.

In some embodiments, the first variable heavy chain domain comprises an amino acid sequence of the variable heavy chain portion of SEQ ID NO: 5, the first variable light chain domain comprises an amino acid sequence of the variable light chain portion of SEQ ID NO: 5, and the portion of the first arm is capable of binding to the portion of the CD45 protein. In some embodiments, the first arm comprises SEQ ID NO: 5, the second arm comprises SEQ ID NO: 9, and the portion of the first arm is capable of binding to the portion of the CD45 protein.

In some embodiments, the first variable heavy chain domain comprises an amino acid sequence of the variable heavy chain portion of SEQ ID NO: 7, the first variable light chain domain comprises an amino acid sequence of the variable light chain portion of SEQ ID NO: 7, and the portion of the first arm is capable of binding to the portion of the CD43 protein. In some embodiments, the first arm comprises SEQ ID NO: 7, wherein the second arm comprises SEQ ID NO: 9, and wherein the portion of the second arm is capable of binding to the portion of the CD43 protein.

In some embodiments, the first variable heavy chain domain comprises an amino acid sequence of the variable heavy chain portion of SEQ ID NO: 28, the first variable light chain domain comprises an amino acid sequence of the variable light chain portion of SEQ ID NO: 28, and the portion of the first arm is capable of binding to the portion of the CD45 protein. In some embodiments, the first arm comprises SEQ ID NO: 28, the second arm comprises SEQ ID NO: 9, and the portion of the first arm is capable of binding to the portion of the CD45 protein. In some embodiments, the first variable heavy chain domain comprises an amino acid sequence of the variable heavy chain portion of SEQ ID NO: 29, the first variable light chain domain comprises an amino acid sequence of the variable light chain portion of SEQ ID NO: 29, and the portion of the first arm is capable of binding to the portion of the CD45 protein. In some embodiments, the first arm comprises SEQ ID NO: 29, the second arm comprises SEQ ID NO: 9, and the portion of the first arm is capable of binding to the portion of the CD45 protein. In some embodiments, the first variable heavy chain domain comprises an amino acid sequence of the variable heavy chain portion of SEQ ID NO: 30, the first variable light chain domain comprises an amino acid sequence of the variable light chain portion of SEQ ID NO: 30, and the portion of the first arm is capable of binding to the portion of the CD45 protein. In some embodiments, the first arm comprises SEQ ID NO: 30, the second arm comprises SEQ ID NO: 9, and the portion of the first arm is capable of binding to the portion of the CD45 protein. In some embodiments, the first variable heavy chain domain comprises an amino acid sequence of the variable heavy chain portion of SEQ ID NO: 31, the first variable light chain domain comprises an amino acid sequence of the variable light chain portion of SEQ ID NO: 31, and the portion of the first arm is capable of binding to the portion of the CD45 protein. In some embodiments, the first arm comprises SEQ ID NO: 31, the second arm comprises SEQ ID NO: 9, and the portion of the first arm is capable of binding to the portion of the CD45 protein. In some embodiments, the first variable heavy chain domain comprises an amino acid sequence of the variable heavy chain portion of SEQ ID NO: 32, the first variable light chain domain comprises an amino acid sequence of the variable light chain portion of SEQ ID NO: 32, and the portion of the first arm is capable of binding to the portion of the CD45 protein. In some embodiments, the first arm comprises SEQ ID NO: 32, the second arm comprises SEQ ID NO: 9, and the portion of the first arm is capable of binding to the portion of the CD45 protein. In some embodiments, the first variable heavy chain domain comprises an amino acid sequence of the variable heavy chain portion of SEQ ID NO: 33, the first variable light chain domain comprises an amino acid sequence of the variable light chain portion of SEQ ID NO: 33, and the portion of the first arm is capable of binding to the portion of the CD45 protein. In some embodiments, the first arm comprises SEQ ID NO: 33, the second arm comprises SEQ ID NO: 9, and the portion of the first arm is capable of binding to the portion of the CD45 protein.

In some embodiments, the portion of the first arm is capable of binding to the portion of the CD3 protein, and wherein the bispecific antibody is capable of clustering a SLAMF6 protein with a core of an immune synapse. In some embodiments, the bispecific antibody is capable of promoting downstream signaling of a SLAMF6 mediated response in a T cell. In some embodiments, the bispecific antibody is capable of enhancing T cell function. In some embodiments, the bispecific antibody is capable of binding to the portion of the CD3 protein and to the portion on the SLAMF6 protein, wherein the CD3 protein and SLAMF6 protein are located on a same T cell.

In some embodiments, the portion of the first arm is capable of binding to the portion of the CD45 protein, and the bispecific antibody is capable of localizing a SLAMF6 protein away from a core of an immune synapse. In some embodiments, the bispecific antibody is capable of disrupting downstream signaling of inhibitory proteins and effector pathways in a T cell. In some embodiments, the bispecific antibody is capable of enhancing T cell function.

In some embodiments, the bispecific antibody is capable of binding to the portion of the CD45 protein and to the portion on the SLAMF6 protein, wherein the CD45 protein and SLAMF6 protein are located on a same T cell.

In some embodiments, the portion of the first arm is capable of binding to the portion of the CD43 protein, and wherein the bispecific antibody is capable of localizing a SLAMF6 protein away from a core of an immune synapse. In some embodiments, the bispecific antibody is capable of disrupting a downstream signaling of inhibitory proteins and effector pathways in a T cell. In some embodiments, the bispecific antibody is capable of enhancing T cell function. In some embodiments, the bispecific antibody is capable of binding to the portion of the CD43 protein and to the portion on the SLAMF6 protein wherein the CD43 protein and SLAMF6 protein are located on a same T cell.

In some embodiments, the SLAMF6 protein is located on a T cell, and wherein the bispecific antibody is capable of promoting binding of a SLAM associated protein (SAP) to an intracellular tail of the SLAMF6 protein on a tumor cell. In some embodiments, the bispecific antibody is capable of inducing a cytokine secretion in a T cell. In some embodiments, the cytokine secretion is a secretion of IL-2.

In some embodiments, the bispecific antibody or a fragment thereof comprises: a third arm comprising a first variable heavy chain domain and a first variable light chain domain, wherein a portion of the first arm is capable of binding to the portion of the CD3 protein, the portion of the CD45 protein, or the portion of the CD43 protein; and a fourth arm comprising a second variable heavy chain domain and a second variable light chain domain, wherein a portion of the second arm is capable of binding to the portion of the SLAMF6 protein. In some embodiments, the bispecific antibody comprises SEQ ID NO: 11 and SEQ ID NO: 16. In some embodiments, the bispecific antibody comprises SEQ ID NO: 12 and SEQ ID NO: 16. In some embodiments, the bispecific antibody comprises SEQ ID NO: 13 and SEQ ID NO: 16. In some embodiments, the bispecific antibody comprises SEQ ID NO: 22 and SEQ ID NO: 16. In some embodiments, the bispecific antibody comprises SEQ ID NO: 23 and SEQ ID NO: 16. In some embodiments, the bispecific antibody comprises SEQ ID NO: 24 and SEQ ID NO: 16. In some embodiments, the bispecific antibody comprises SEQ ID NO: 25 and SEQ ID NO: 16. In some embodiments, the bispecific antibody comprises SEQ ID NO: 26 and SEQ ID NO: 16. In some embodiments, the bispecific antibody comprises SEQ ID NO: 27 and SEQ ID NO: 16.

### Definitions

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

As used herein, the term “antibody” includes synthetic antibodies, monoclonal antibodies, oligoclonal or polyclonal antibodies, multiclonal antibodies, recombinantly produced antibodies, intrabodies, monospecific antibodies, monovalent antibodies, multispecific antibodies, multivalent antibodies, bispecific antibodies, bivalent antibodies, tetravalent antibodies, human antibodies, humanized antibodies, chimeric antibodies, CDR-grafted antibodies, primatized antibodies, Fab fragments, F(ab′) fragments, F(ab′)2 fragments, Fv fragments, single-chain FvFcs (scFv-Fc), single-chain Fvs (scFv), Dabs, nanobodies, anti-idiotypic (anti-Id) antibodies, and any other immunologically-reactive/antigen-binding fragments thereof. The term “immunoglobulin” (Ig) is used interchangeably with “antibody” herein.

The term “bispecific antibody” refers to an antibody having specificities for at least two different, typically non-overlapping, epitopes. Such epitopes may be on the same or different targets. If the epitopes are on different targets, such targets may be on the same cell or different cells or cell types. In some embodiments, the bispecific antibody comprises a first and second chain. In some embodiments, the first chain comprises an scFv with specificity for a first epitope and the second chain comprises an scFv with specificity for a second epitope. In some embodiments, the first and second chains each further comprise a Fc domain. In some embodiments, the first chain comprises an scFv with specificity for a first epitope, an Fc domain, and a second scFV with specificity for a second epitope and the second chain comprises an scFv with specificity for the first epitope, and Fc domain, and a second scFV with specificity for the second epitope. In some embodiments, the bispecific antibody comprises a first and a second heavy chain. In some embodiments, the bispecific antibody comprises a first and a second heavy chain and a first and a second light chain. In some embodiments, the bispecific antibody comprises any of the designs described in FIG. 2 of Brinkmann U, Kontermann R E, The making of bispecific antibodies, MAbs, 2017 February/March, 9(2):182-212, PMID: 28071970 the content of which is hereby incorporated by reference in its entirety. In some embodiments, the bispecific antibody binds to CD3 and SLAMF6, binds to CD45 and SLAMF6, or binds to CD43 and SLAMF6.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein, the term “subject” refers to a vertebrate animal. In one embodiment, the subject is a mammal or a mammalian species. In one embodiment, the subject is a human. In one embodiment, the subject is a healthy human adult. In other embodiments, the subject is a non-human vertebrate animal, including, without limitation, non-human primates, laboratory animals, livestock, racehorses, domesticated animals, and non-domesticated animals. In one embodiment, the term “human subjects” means a population of healthy human adults.

### BRIEF DESCRIPTION OF THE DRAWINGS

The following figures depict illustrative embodiments of the invention.

FIGS. 1A-H show that separation of SLAMF6 and CD3 inhibits T cell proliferation. (A) A schematic representation of the assays used to study SLAMF6 compartmentation. T cells were stimulated with either immobilized anti-CD3 (αCD3) and anti-SLAMF6 (αSLAMF6) on a plate surface (top) or immobilized αCD3 but soluble αSLAMF6 (bottom). (Bi-Bii) Freshly isolated primary CD3⁺ T cells were stained with CFSE and then cultured in the presence of plate-coated αCD3 or plate-coated αCD3+αSLAMF6 or plate-coated αCD3+soluble αSLAMF6. After 120 h, the cells were assayed for FITC fluorescence for three independent experiments (n=3). The data were analyzed for percent (%) of proliferating cells as depicted (black line); plate-coated αCD3+αSLAMF6 resulted in greater proliferation as compared with αCD3 alone, whereas the addition of soluble αSLAMF6 inhibited proliferation. (Ci-Cii) CD25 and PD-1 expression at 120 h was analyzed using flow cytometry. (D, E) Freshly isolated primary CD3 T cells were cultured in the presence of plate-coated αCD3+αSLAMF6 or plate-coated αCD3+soluble αSLAMF6. (D, E) The supernatant was harvested, and IL-2 levels at different time intervals over 96 h and (E) IFN-γ levels at 48 h were analyzed by ELISA. (F) Jurkat T cells were stimulated in the presence of brefeldin for 6 h, after which intracellular IL-2 was analyzed by flow cytometry. This experiment was repeated twice (n=2). (Gi-Gii) Freshly isolated primary CD3 T cells were cultured as above for 120 h. Cell differentiation was analyzed based on cell surface expression of CD45RA and CCR7. A weighed T cell maturation index was calculated as (1*Naïve+2*Central Memory+3*Effector Memory+4*Terminal Effector Memory)/4. This experiment was repeated twice with the average value shown here. (H) The Cell number was assessed by automated counting every 24 h. The experiment was done in triplicate (n=3). *P≤0.05 for an unpaired t test.

FIGS. 2Ai-C show that SLAMF6 and TCR signaling complexes share key mediators. V5-SLAMF6-expressing Jurkat T cells were stimulated with plate-coated αCD3+αSLAMF6 (plate) or plate-coated αCD3+soluble αSLAMF6 (soluble) for 15 min. The cells were then lysed and the lysate mixed with V5-coupled agarose beads to enrich for V5-tagged SLAMF6 immunoprecipitation. Pull-down lysate proteins were separated by electrophoresis and submitted for mass spectrometry analysis. (Ai-Aii) Protein enrichment pathway analysis for the plate versus soluble stimulation conditions was performed. (B) Proteins interacting with SLAMF6 were identified, listed by peptide-spectrum match score. (C) A schematic demonstrating that the SLAMF6-interacting proteins identified in the immunoprecipitation (marked by a purple star) are known to be essential for proximal TCR signal transduction, emphasizing the interconnection between the two receptors and their signaling interactome. Three independent experimental repeats were performed. (n=3).

FIGS. 3A-D show that SLAMF6 clustering in the immunologic synapse (IS) enhances cytokine secretion. (A) A schematic representation of SLAMF6 in the absence (top) or presence (bottom) of homophilic receptor ligation in a T-B cell co-culture. (B) Jurkat T cells were transfected via nucleofection with LifeAct mCherry and SLAMF6 GFP, then co-cultured with Raji B Cell APCs. Images are representative of at least 30 cells from at least two independent experiments. The scale bar is 5 μm. Actin clearance was defined as a mature IS. (C) Jurkat T cells were treated with αCD3-conjugated beads and αSLAMF6-conjugated beads versus αCD3+αSLAMF6-conjugated beads (both antibodies on the same bead). After 24 h, the supernatant was harvested, and ELISA analyzed IL-2 levels for at least three independent experiments (n=3). (D) Jurkat T cells were treated with αCD3+αSLAMF6 or αCD3+αSLAMF6+cross-linker for 24 h, after which the supernatant was harvested and ELISA analyzed IL-2 levels for at least three independent experiments (n=3). *P<0.05 for an unpaired t test.

FIGS. 4A-E show that anti-CD3/SLAMF6-bispecific antibody clusters SLAMF6 to the CD3 and augments T cell activation. (A) A schematic representation of anti-CD3/SLAMF6-bispecific antibody (αCD3/SLAMF6) binding and clustering the two receptors together. (B) αCD3/SLAMF6 antibody binding was validated using an ELISA assay: αCD3 binding was assessed against immobilized SLAMF6 KO Jurkat T cells, whereas anti-SLAMF6 binding was assessed against immobilized Raji B cells. (C) Jurkat T cells were treated with αCD3, αCD3+soluble αSLAMF6, or αCD3/SLAMF6 at three different concentrations of the bispecific antibody: 0.1, 1, and 10 μg/ml. After 24 h, IL-2 levels were analyzed by ELISA for at least two independent experiments (n=2). (D) Raji B cells were preloaded with increasing concentrations of SEE and co-cultured with Jurkat T cells in the absence (blue) or presence (magenta) of 1 μg/ml of αCD3/SLAMF6 antibody. IL-2 levels were analyzed for at least two independent experiments (n=2). (E) Raji B cells were preloaded with SEE and co-cultured with Jurkat T cells at increasing concentrations of the αCD3/SLAMF6 antibody. IL-2 levels were analyzed for at least two independent experiments (n=2). *P<0.05, **P<0.01, ***P<0.001 for an unpaired t test.

FIGS. 5A-G show that anti-CD45/SLAMF6-bispecific antibody inhibits SLAMF6 enrichment in the synapse but still augments T cell activation. (A) A schematic representation of anti-CD45/SLAMF6 (CD45/SLAMF6) binding to inhibit SLAMF6 clustering with CD3 in the IS. (B) αCD45/SLAMF6 antibody binding was quantified using an ELISA assay: αCD45 and αSLAMF6 binding was assessed against immobilized, recombinant ectodomains of CD45 and SLAMF6, respectively. (Ci-Cii) Jurkat T cells were transfected with GFP-tagged SLAMF6, and Raji B cells were stained with LifeAct Far Red and preloaded with SEE (2 ng/ml). Jurkat T cells were then co-cultured with Raji B cells for 30 min. Synapse formation with enrichment of SLAMF6 in the IS was visualized (top row). To visualize the distribution of CD45, we next transfected the Jurkat T cells with OFPSpark-tagged CD45 and GFP-tagged SLAMF6. In the Jurkat T-Raji B co-cultures, the exclusion of CD45 was coupled with the enrichment of SLAMF6 in the IS (middle row). Finally, we pretreated Jurkat T cells with anti-CD45/SLAMF6 10 μg/ml for 15 min (bottom image). The exclusion of CD45 was now associated with a lack of enrichment of SLAMF6 in the IS (bottom row). Images are representative of at least 40 cell conjugates per each experimental condition from two independent experiments. The scale bar is 5 μm. The percentage of cell conjugates with SLAMF6 enrichment in the IS was quantified; results are summarized in the bar graph. (D) Jurkat T cells were treated with αCD3 and αCD45/SLAMF6 at three concentrations: 0.1, 1, and 10 μg/ml of the bispecific antibody. After 24 h, IL-2 levels were analyzed by ELISA. (E) Raji B cells were preloaded with different concentrations of SEE and co-cultured with Jurkat T cells in the absence (blue) or presence (magenta) of 1 μg/ml of αCD45/SLAMF6 antibody. IL-2 levels were analyzed for at least three independent experiments (n=3). (F) Raji B cells were preloaded with SEE and co-cultured with T cells at increasing concentrations of αCD45/SLAMF6 antibody. IL-2 levels were analyzed for at least three independent experiments (n=3). (G) Raji B cells and Jurkat T cells were co-cultured either in the presence of, or after T cell pretreatment with, αCD45/SLAMF6. Specifically, in the first experimental condition, αCD45/SLAMF6 was added as Jurkat T-Raji B conjugates formed (supporting in trans antibody ligation). In contrast, in the second experimental condition, Jurkat T cells were pretreated with αCD4/SLAMF6 for 30 min, washed, and subsequently co-cultured with the Raji B cells, supporting in cis antibody ligation on T cells before addition of the B cells. IL-2 levels were analyzed for at least three independent experiments (n=3). *P<0.05 for an unpaired t test.

FIGS. 6A-Diii show that anti-CD45-SLAMF6 antibody enhances T cell response in primary human cell-based assay. PBMCs were treated with SEE and three different concentrations of either the monovalent (αCD45/SLAMF6) or the bivalent (αCD45-Ig-SLAMF6) anti-CD45/SLAMF6-bispecific antibody. (A, B) After 24 h, (A) IL-2 and (B) IFN-7 levels were measured by ELISA. The results of at least three independent experiments (n=3) are shown. (C, D) Primary human CD3-positive T cells were isolated from whole blood and cultured with anti-CD3 in the presence αCD45/SLAMF6. (C) After 5 min, the cells were analyzed for phosphorylation of CD3 ζ chain using flow cytometry. (Di-Diii) After 24 h, the cells were analyzed for CD69 expression, and the supernatant was analyzed for IL-2 and IFN-γ release. *P<0.05, **P<0.01 for an unpaired t test.

FIGS. 7A-D show a gating strategy for flow cytometry. (A, B) T cells were identified on the FSC-SSC plot followed by (B) selection of single cells. (C) Live cells were identified as those that did not take up UV Zombie dye. (D) CD4 and CD8 T cell populations were identified. For all subsequent markers, FMOs were used to identify the negative populations.

FIGS. 8A-B show that protein-protein interactions downstream of SLAMF6 activation differ based on stimulation condition. V5-SLAMF6-expressing Jurkat T cells were stimulated with plate-coated αCD3+αSLAMF6 (plate) or plate-coated αCD3+soluble αSLAMF6 (soluble) for 15 min. The cells were then lysed and the lysate mixed with V5-coupled agarose beads to enrich for V5-tagged SLAMF6 immunoprecipitation. Pull-down lysate proteins were separated by electrophoresis and submitted for mass spectrometry analysis. (A, B) Protein-protein interaction identified prediction models of kinases involved downstream of SLAMF6 signaling in the plate (A) and soluble (B) conditions. Kinases that were validated in the pull-down are shown in red.

FIG. 9 shows confirming purity of synthesized antibodies by electrophoresis. Bispecific antibodies, anti-CD3/SLAMF6 and anti-CD45/SLAMF6, were supplemented with 1 M HEPS buffer, and purity was determined by running PAGE gel against 1 μg of BSA as a control.

FIGS. 10A-B shows a schematic of two bispecific antibodies. (A) shows a schematic of an exemplary bispecific antibody that is monovalent for each target (i.e., it bivalent). (B) shows a schematic of an exemplary bispecific antibody that is bivalent for each target (i.e., it is tetravalent).

FIGS. 11A-B show binding curves to human-CD45RO-ECD-His-coated plates was quantified by ELISA. FIG. 11A shows binding curves for anti-CD45 antibody clones 023, 026, 027, and 028, CD45RO Monoclonal Antibody (UCHL1) (eBioscience™), anti-HEL-human IgG1 isotype control, and blank. FIG. 11B shows binding curves for anti-CD45 antibody clones 031, and 042, CD45RO Monoclonal Antibody (UCHL1) (eBioscience™), anti-HEL-human IgG1 isotype control, and blank. EC50 values were calculated with GraphPad Prism (v10.2.1).

FIGS. 12A-F shows SRP analysis of binding of anti-CD45 antibodies to captured human-CD45RO-ECD-His. FIG. 12A shows binding of CD45RO Monoclonal Antibody (UCHL1) to captured human-CD45RO-ECD-His. FIG. 12B shows binding of anti-CD45 antibody clone 23 to captured human-CD45RO-ECD-His. FIG. 12C shows binding of anti-CD45 antibody clone 26 to captured human-CD45RO-ECD-His. FIG. 12D shows binding of anti-CD45 antibody clone 27 to captured human-CD45RO-ECD-His. FIG. 12E shows binding of anti-CD45 antibody clone 28 to captured human-CD45RO-ECD-His. FIG. 12F shows binding of anti-CD45 antibody clone 31 to captured human-CD45RO-ECD-His.

FIG. 13 shows binding of anti-CD45 antibodies to cell-expressed CD45 in Jurkat cells by flow cytometry. FIG. 13 shows binding for anti-CD45 antibody clones 027, 042, 028, 031, 026, and 023 compared to unstained, sec-only, and non-relevant control.

FIG. 14 shows binding of anti-CD45 antibodies to cell-expressed CD45 in Raji cells by flow cytometry. FIG. 14 shows binding for anti-CD45 antibody clones 027, 042, 028, 031, 026, and 023 compared to unstained, sec-only, and non-relevant control.

### DETAILED DESCRIPTION

Disclosed herein are novel bispecific antibodies that in some embodiments are useful for preventing or treating disease (e.g., cancer) or infection (e.g., human immunodeficiency virus (HIV)). Described herein are novel anti-CD3×SLAMF6, CD45×SLAMF6, and anti-CD43×SLAMF6 bispecific antibodies that in some embodiments are advantageous over existing anti-CD3, anti-CD45, anti-CD43, and anti-SLAMF6 monospecific antibodies for prevention or treatment of one or more cancers. In some embodiments, the novel anti-CD3×SLAMF6, anti-CD45×SLAMF6, and anti-CD43×SLAMF6 bispecific antibodies inhibit PD-1 signaling and enhance T cell activation to a greater extent than anti-CD3, anti-CD45, anti-CD43, and anti-SLAMF6 monospecific antibodies separately or in combination.

### T Cells and The Immune Synapse

T cells have surface receptors that can act as immune checkpoint receptors, such as PD-1. See Speiser D E, Ho P C, Verdeil G. Regulatory Circuits of T Cell Function in Cancer. Nat Rev Immunol. 2016 October; 16(10): pp. 599-611, incorporated by reference in its entirety herein. These receptors act as “checkpoints” to prevent excessive immune activation. However, cancer cells can exploit these checkpoint pathways to evade immune detection and attack. See Liu J, Chen Z, Li Y, Zhao W, Wu J and Zhang Z. PD-1/PD-L1 Checkpoint Inhibitors in Tumor Immunotherapy. Front. Pharmacol. 2021 September; pp. 12: p. 731798, incorporated by reference in its entirety herein. PD-1, is expressed on the surface of activated T cells while its ligands PD-L1 and PD-L2 are expressed on various cancer cells. See Liu, et al. (2021). The immune synapse is the interface between T cells and tumor cells. This interface, or microenvironment, includes the checkpoint receptors (e.g., PD-1, PDL-1, PDL-2) and other immune receptors and ligands needed for T cells to function. The immune synapse is organized into three compartments; every protein has a specific location. For example, with respect to proteins found on T cells, the T cell receptor (TCR) is localized to the center of the synapse, PD-1 and CD28 are located in the peripheral synapse, and LFA-1, CD43, and CD45 are found in the distal compartment of the synapse. See e.g., Delon J, Kaibuchi K, Germain R N. Exclusion of CD43 from the immunological synapse is mediated by phosphorylation-regulated relocation of the cytoskeletal adaptor moesin. Immunity. 2001 November; 15(5):691-701. doi: 10.1016/s1074-7613(01)00231-x. PMID: 11728332. This organization is critical for the function of the synapse, where specific clusters of proteins are formed.

Cancer immunotherapy drugs work by blocking these checkpoint receptors on T cells, thereby unleashing the immune system to recognize and destroy cancer cells more effectively. He X, Xu C. Immune Checkpoint Signaling and Cancer Immunotherapy. Cell Res. 2020 August; 30(8): pp. 660-669, incorporated by reference in its entirety herein. For example, PD-1 inhibitors prevent PD-1 on T cells from binding to its ligands PDL-1 and PDL-2 on tumor cells, thereby preventing the tumor cells from evading the T cell immune response. However, some individuals experience immune-related adverse events (irAEs) from non-specific activation of T cells.

An alternate target for cancer immunotherapy is SLAMF6. SLAMF6 is expressed on a variety of hematopoietic cells including T cells, and it is important in fine tuning antigen driven immune responses. Gartshteyn Y, Askanase A D, Mor A, SLAM Associated Protein Signaling in T cell: Tilting the Balance Toward Autoimmunity. Front Immunol. 2021 Apr. 16; 12:654839. As part of its role in T cell activation, SLAMF6 binds with the intracellular protein SLAM associated protein (SAP) at the immune tyrosine switch motif (ITSM) domain of its cytoplasmic tail. Following SAP binding, lymphocyte specific protein tyrosine kinase (LCK) and proto-oncogene tyrosine protein kinase (FYN) are recruited to phosphorylate SLAMF6's cytoplasmic tail and bridge the signal from SLAMF6 with a CD3 T cell receptor signal at the immune synapse, promoting phosphorylation of the CD3 zeta chain and recruitment of zeta chain of T cell receptor associated protein kinase 70 (ZAP70), resulting in a net positive activation signal and release of IL-2 and IL-4.

SLAMF6 activity affects T-cell functions and can modulate the anticancer immune responses suggesting potential for pharmaceutical targeting in cancer immunotherapy. Eisenberg, G., et al., Soluble SLAMF6 Receptor Induces Strong CD8(+) T-cell Effector Function and Improves Anti-Melanoma Activity In Vivo. Cancer Immunol Res, 2018. 6(2): p. 127-138; Schenkel, J. M., et al., Conventional type I dendritic cells maintain a reservoir of proliferative tumor-antigen specific TCF-1(+) CD8(+) T cells in tumor-draining lymph nodes. Immunity, 2021. 54(10): p. 2338-2353 e6. However, the exact role of SLAMF6 in T-cell signaling is not well understood and contradictory reports of SLAMF6 enhancing or dampening T-cell activity have been described. Yigit, B., et al., SLAMF6 as a Regulator of Exhausted CD8(+) T Cells in Cancer. Cancer Immunol Res, 2019. 7(9): p. 1485-1496; Hajaj, E., et al., SLAMF6 deficiency augments tumor killing and skews toward an effector phenotype revealing it as a novel T cell checkpoint. Elife, 2020. 9.

In vitro antibody ligation of SLAMF6 cross-linked with activated CD3 results in increased T cell proliferation and interferon gamma (IFN-γ) release, suggesting that SLAMF6 is a co-stimulatory receptor. Valdez, P. A., et al., NTB-A, a new activating receptor in T cells that regulates autoimmune disease. J Biol Chem, 2004. 279(18): p. 18662-9. It was also previously demonstrated that SLAMF6 mediated T-cell activation is dependent on the clustering of SLAMF6 endodomain with CD3, highlighting that spatial proximity of the receptors and their downstream signaling molecules within the IS is necessary for T-cell activation. Dragovich, M. A., et al., SLAMF6 clustering is required to augment T cell activation. PLoS One, 2019. 14(6): p. e0218109.

Without intending to be bound by any particular theory, the design of antibodies for the prevention or treatment of cancer is improved by taking into account the localization of SLAMF6 on the surface of the immune cells in the context of the immune synapse. Without intending to be bound by any particular theory, it is hypothesized that clustering SLAMF6 with CD3 at the immune synapse promotes T cell activation and disrupts PD-1 mediated inhibitory signals, and localizing SLAMF6 with CD45 or CD43 away from the immune synapse draws with it proteins necessary for inhibition of T cell activation. Additionally, without intending to be bound by any particular theory, localizing SLAMF6 with CD45 or CD43 away from the immune synapse can avoid activating all T cells and instead only activate those T cells forming a synapse with a cancer cell, reducing the risk of nonspecific T cell activation and immune related adverse events. Thus, changing the location of SLAMF6 within the different compartments of the immune synapse could serve as an alternative, efficient, and safer approach to treating cancer patients.

Described herein are improved versions of SLAMF6 antibodies. A panel of novel anti-CD3×SLAMF6 bispecific antibodies, anti-CD45×SLAMF6 bispecific antibodies, and anti-CD43×SLAMF6 bispecific antibodies were designed and are described herein. Without intending to be bound by any particular theory, the rationale behind the design of the bispecific antibodies disclosed herein was that disruption of selected protein interactions through altered localization would serve as a strategy to affect T cell function better. The SLAMF6 pathway provides a clear example to demonstrate how changing the localization of a protein on the cell surface can impact T cell function. Without intending to be bound by any particular theory, it is hypothesized that clustering SLAMF6 with CD3 at the immune synapse via bispecific antibodies promotes T cell activation and disrupts PD-1 mediated inhibitory signals, and localizing SLAMF6 with CD45 or CD43 away from the immune synapse via bispecific antibodies draws with it proteins necessary for inhibition of T cell activation. Additionally, without intending to be bound by any particular theory, localizing SLAMF6 with CD45 or CD43 away from the immune synapse can avoid activating all T cells and instead only activate those T cells forming a synapse with a cancer cell, reducing the risk of nonspecific T cell activation and immune related adverse events. Without intending to be bound by any particular theory, it is hypothesized that a bispecific antibody to CD3 would bind to CD3, which would serve as an anchor, within the synapse, drawing SLAMF6 to the core of the synapse. Similarly, it is hypothesized that a bispecific antibody to CD45 (or to CD43) and to SLAMF6 would bind to CD45 (or CD43), which would serve as an anchor, outside the synapse, and pull SLAMF6 away from the core of the synapse. In some embodiments, the novel bispecific antibodies disclosed herein bind to SLAMF6 and CD3 on the same cells (binding in cis) and not across cells (binding in trans, i.e., where one arm of the antibody binds to SLAMF6 on one cell, and the other arm of the antibody binds to CD3 on another cell). In some embodiments, the novel bispecific antibodies disclosed herein bind to SLAMF6 and CD45 on the same cells (binding in cis) and not across cells (binding in trans, i.e., where one arm of the antibody binds to SLAMF6 on one cell, and the other arm of the antibody binds to CD45 on another cell). In some embodiments, the novel bispecific antibodies disclosed herein bind to SLAMF6 and CD43 on the same cells (binding in cis) and not across cells (binding in trans, i.e., where one arm of the antibody binds to SLAMF6 on one cell, and the other arm of the antibody binds to CD43 on another cell).

In some embodiments, described herein are anti-CD3×SLAMF6, anti-CD45×SLAMF6, and anti-CD43×SLAMF6 bispecific antibodies for addressing the limitation of SLAMF6 localization. In some embodiments, the anti-CD3×SLAMF6 bispecific antibody described herein functions by binding to CD3 and pulling SLAMF6 to the core of the synapse, promoting downstream signaling of SLAMF6 and effector pathways, and/or enhancing T-cell function. In some embodiments, the anti-CD45×SLAMF6 bispecific antibody described herein functions by binding to CD45 and pulling SLAMF6 away from the core of the synapse, disrupting downstream signaling of inhibitory proteins such as PD-1 and effector pathways, and/or enhancing T-cell function. In some embodiments, the anti-CD43×SLAMF6 bispecific antibody described herein functions by binding to CD43 and pulling SLAMF6 away from the core of the synapse, disrupting downstream signaling of inhibitory proteins such as PD-1 and effector pathways, and/or enhancing T-cell function.

In some embodiments, disclosed herein are anti-CD3×SLAMF6 bispecific antibodies that bind to CD3 and cluster SLAMF6 in the core of the synapse. In some embodiments, clustering SLAMF6 in the core of the synapse disrupts downstream signaling of inhibitory proteins such as PD-1 and effector pathways, and enhanced T-cell function. In some embodiments, disclosed herein are anti-CD45×SLAMF6 bispecific antibodies that bind to CD45 and localize SLAMF6 away from the core of the synapse. In some embodiments, disclosed herein are anti-CD43×SLAMF6 bispecific antibodies that bind to CD43 and localize SLAMF6 away from the core of the synapse. In some embodiments, localizing SLAMF6 away from the core of the synapse disrupts downstream signaling of inhibitory proteins such as PD-1 and effector pathways, and enhances T-cell function.

In some embodiments, the use of anti-CD3×SLAMF6 bispecific antibodies disclosed herein have one or more of the following advantages over monospecific antibodies: 1) they cluster SLAMF6 to the core of the synapse and thereby inhibit PD-1 mediated inhibitory responses, 2) they promote binding of a SLAM associated protein (SAP) to an intracellular tail of the SLAMF6 protein, 3) the bispecific antibody enhancement of T cell function is greater than either monospecific antibody does individually or in a nonspecific combination mixture, and 4) the bispecific antibody enhancement of T cell function is dose-dependent. In some embodiments, using one or more of the innovative bispecific antibodies describes herein offer a more potent and safer technology to treat cancer patients resistant to current immunotherapies.

In some embodiments, the use of anti-CD45×SLAMF6 and/or anti-CD43×SLAMF6 bispecific antibodies disclosed herein have one or more of the following advantages over monospecific antibodies: 1) they localize SLAMF6 away from the synapse and thereby removing inhibitory factors from the core of the synapse, 2) they promote binding of a SLAM associated protein (SAP) to an intracellular tail of the SLAMF6 protein, 3) the bispecific antibody enhancement of T cell function is greater than either monospecific antibody does individually or in a nonspecific combination mixture, and 4) the bispecific antibody enhancement of T cell function is dose-dependent. In some embodiments, using one or more of the innovative bispecific antibodies describes herein offer a more potent and safer technology to treat cancer patients resistant to current immunotherapies.

In some embodiments, an anti-CD3×SLAMF6, anti-CD45×SLAMF6, and/or an anti-CD43×SLAMF6 bispecific antibody, as disclosed herein, is used to prevent or treat cancer in a subject. In some embodiments, the cancer is selected from colorectal cancer, lung cancer, bladder cancer, breast cancer, cervical cancer, kidney cancer, leukemia, Hodgkin lymphoma, non-Hodgkin lymphoma, prostate cancer, skin cancer (e.g., melanoma), head and neck cancer, endometrial cancer, colon cancer, rectal cancer, liver cancer, thyroids cancer, esophageal cancer, renal cell cancer, and a combination thereof. In some embodiments, an anti-CD3×SLAMF6, anti-CD45×SLAMF6, and/or an anti-CD43×SLAMF6 bispecific antibody, as disclosed herein, is used to prevent or treat disease caused by any solid tumor that is not able to repair errors in its DNA that occur when the DNA is copied.

In some embodiments, an anti-CD3×SLAMF6, anti-CD45×SLAMF6, and/or an anti-CD43×SLAMF6 bispecific antibody, as disclosed herein, is used to prevent or treat an autoimmune disease. In some embodiments, the autoimmune disease is Lupus in a subject.

### Antibodies

There are five classes of human antibodies (i.e., IgA, IgD, IgE, IgG, and IgM) and each have various isotypes (e.g., IgG1, IgG2, IgG3, IgG4, IgA1, and IgA2). In some embodiments, the antibodies disclosed herein belong to the IgG class. IgG can be further divided into four subclasses: IgG1, IgG2, IgG3, and IgG4. Each subclass has a unique profile with respect to antigen binding, immune complex formation, complement activation, triggering of effector cells, half-life, and placental transport. E.g., see Gestur Vidarsson, et al., IgG Subclasses and Allotypes: From Structure to Effector Functions, 5 Frontiers in Immunology 520 (2014), incorporated by reference herein in its entirety.

The IgG immunoglobulin molecule consists of four polypeptide chains, two identical light (L) chains and two identical heavy (H) chains. The four chains are joined by disulfide bonds in a “Y” configuration wherein the light chains bracket the heavy chains starting at the mouth of the “Y” and continuing through the variable region to the dual ends of the “Y”. Each L chain is linked to an H chain by one covalent disulfide bond, while the two H chains are linked to each other by one or more disulfide bonds depending on the H chain isotype. Each H and L chain also has regularly spaced intrachain disulfide bridges. Each heavy chain consists of an N-terminal variable domain (VH) and three constant domains (CH1, CH2, CH3), with an additional “hinge region” between CH1 and CH2. Similarly, the light chains consist of an N-terminal variable domain (VL) and a constant domain (CL). The variable domains of the heavy chain and light chain may be referred to as “VH” and “VL”, respectively. These domains are generally the most variable parts of the antibody (relative to other antibodies of the same class) and contain the antigen binding sites. The VL is aligned with the VH and the CL is aligned with the first constant domain of the heavy chain (CH1). The pairing of a VH and VL together forms a single antigen-binding site. The part of the antibody formed by the lower hinge region and the CH2/CH3 domains of the heavy chain is called “Fc” (“fragment crystalline”). See e.g., Basic and Clinical Immunology, 8th Edition, Daniel P. Sties, Abba I. Terr and Tristram G. Parsolw (eds), Appleton & Lange, Norwalk, CT, 1994, page 71 and Chapter 6, incorporated by reference herein in its entirety.

The variability in an antibody sequence is concentrated in three segments called complementarity determining regions (CDRs) (also called hypervariable regions (HVRs)) both in the light-chain and the heavy-chain variable domains. The more highly conserved portions of variable domains are called the framework regions (FR). The variable domains of native heavy and light chains each comprise four FRs, largely adopting a beta-sheet configuration, connected by three CDRs, which form loops connecting, and in some cases forming part of, the beta-sheet structure. The CDRs in each chain are held together in close proximity by the FRs and, with the CDRs from the other chain, contribute to the formation of the antigen binding site of antibodies. See Kabat et al, Sequences of Immunological Interest, Fifth Edition, National Institute of Health, Bethesda, MD (1991), incorporated by reference in its entirety herein. The constant domains are not involved directly in the binding of antibody to an antigen, but exhibit various effector functions, such as participation of the antibody in antibody-dependent cellular toxicity.

By way of example, CDRs may be defined using the nomenclature described by Kabat et al. (1991, NIH Publication 91-3242, National Technical Information Service, Springfield, Va.), incorporated by reference in its entirety herein. Specifically, residues 31-35 (CDR-H1), 50-65 (CDR-H2), and 95-102 (CDR-H3) in the heavy chain variable region and residues 24-34 (CDR-L1), 50-56 (CDR-L2), and 89-97 (CDR-L3) in the light chain variable region.

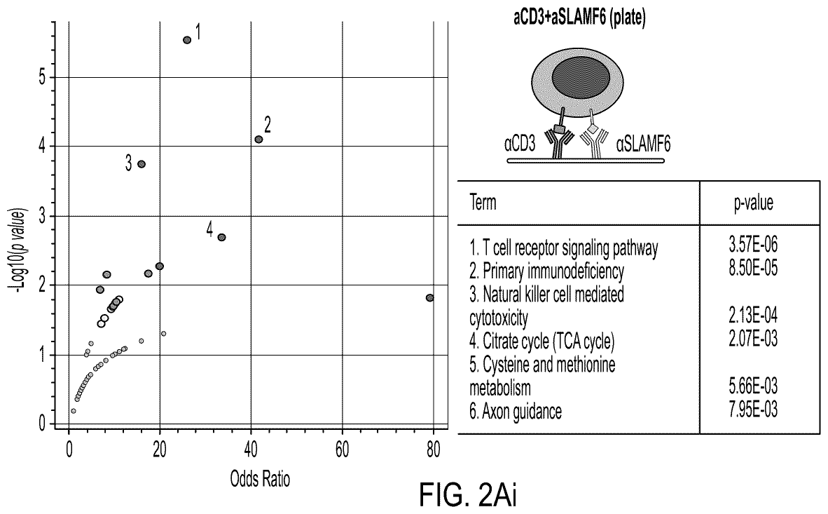

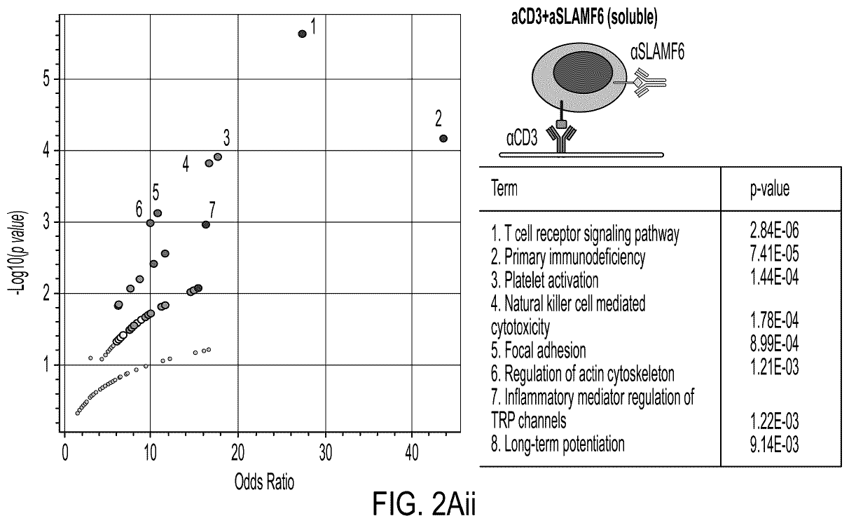

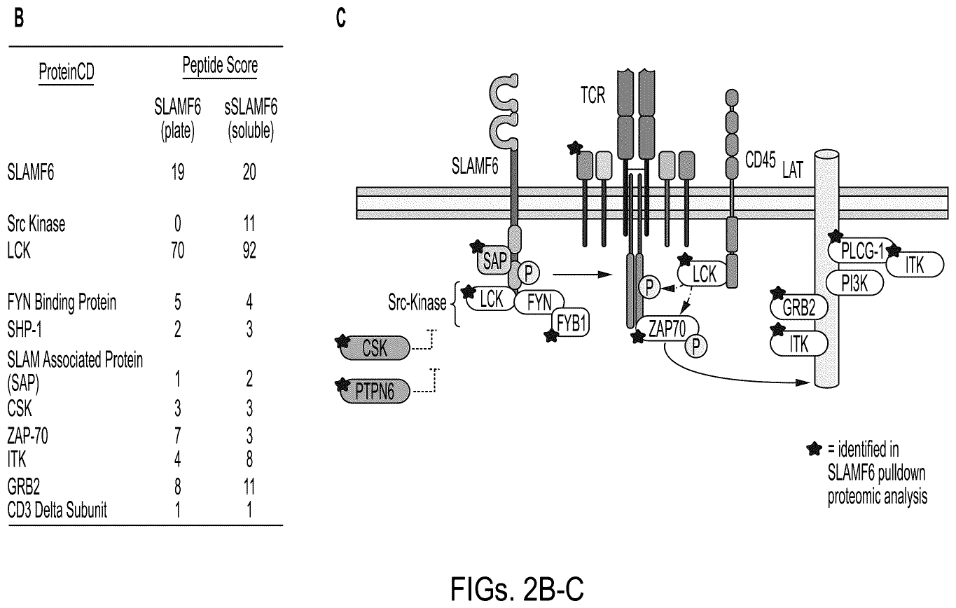

However, the antibodies disclosed herein (e.g., bispecific antibodies) are not limited to full-length antibodies. The antibodies of the various embodiments disclosed herein can include one or more of synthetic antibodies, monoclonal antibodies, oligoclonal or polyclonal antibodies, multiclonal antibodies, recombinantly produced antibodies, intrabodies, monospecific antibodies, monovalent antibodies, multispecific antibodies, multivalent antibodies, bispecific antibodies, bivalent antibodies, tetravalent antibodies, human antibodies, humanized antibodies, chimeric antibodies, CDR-grafted antibodies, primatized antibodies, Fab fragments, F(ab′) fragments, F(ab′)2 fragments, Fv fragments, single-chain FvFcs (scFv-Fc), single-chain Fvs (scFv), Dabs, nanobodies, anti-idiotypic (anti-Id) antibodies, and any other immunologically-reactive/antigen-binding fragments thereof. In some embodiments, the antibody is a bispecific antibody. In some embodiments, the bispecifc antibody comprises a first and second chain. In some embodiments, the first chain comprises an scFv with specificity for a first epitope and the second chain comprises an scFv with specificity for a second epitope. In some embodiments, the first and second chains each further comprise a Fc domain. In some embodiments, the first chain comprises an scFv with specificity for a first epitope, an Fc domain, and a second scFV with specificity for a second epitope and the second chain comprises an scFv with specificity for the first epitope, and Fc domain, and a second scFV with specificity for the second epitope. In some embodiments, the bispecific antibody comprises a first and a second heavy chain and a first and a second light chain. The pairing of the first VH and first VL together forms a single antigen-binding site specific for a first epitope and the pairing of the second VH and second VL together forms a single antigen-binding site specific for a second epitope. Such epitopes may be on the same or different targets. If the epitopes are on different targets, such targets may be on the same cell or different cells or cell types. In some embodiments, the bispecific antibody comprises a first and a second heavy chain and does not comprise any light chains, wherein the first VH forms a single antigen-binding site specific for a first epitope and the second VH forms a single antigen-binding site specific for a second epitope. Such epitopes may be on the same or different targets. If the epitopes are on different targets, such targets may be on the same cell or different cells or cell types. In some embodiments, the bispecific antibody comprises any of the designs described in FIG. 2 of Brinkmann U, Kontermann R E, The making of bispecific antibodies, MAbs, 2017 February/March, 9(2):182-212, PMID: 28071970 the content of which is hereby incorporated by reference in its entirety.

In some embodiments, the bispecific antibody is a tetravalent antibody comprising a first and a second heavy chain and a first and a second light chain N-terminal to an Fc region of an antibody, and a third and a fourth heavy chain and a third and a fourth light chain C-terminal to an Fc region of an antibody. In some embodiments, the first and the second heavy chains and the first and the second light chains contain portions that bind to a portion of a first epitope, and the third and the fourth heavy chains and the third and the fourth light chains contain portions that bind to a portion of a second epitope. In some embodiments, the first and the third heavy chains and the first and the third light chains contain portions that bind to a portion of a first epitope, and the second and the fourth heavy chains and the second and the fourth light chains contain portions that bind to a portion of a second epitope. In some embodiments, the bispecific antibody is a tetravalent antibody comprising, a first chain comprising an scFv with specificity for a first epitope, an Fc domain, and a second scFV with specificity for a second epitope and the second chain comprises an scFv with specificity for the first epitope, and Fc domain, and a second scFV with specificity for the second epitope.

In some embodiments, the bispecific antibodies disclosed herein contain various modifications, substitutions, additions, or deletions to the variable or binding regions of one or more arms of an anti-CD3×SLAMF6 antibody, anti-CD45×SLAMF6 antibody, or an anti-CD43×SLAMF6 antibody disclosed herein. In some embodiments, the bispecific antibodies disclosed herein may contain substitutions or modifications of the constant region (i.e., the Fc region). The antibodies disclosed herein may contain one or more additional amino acid residue substitutions, mutations and/or modifications, which result in a compound with preferred characteristics including, but not limited to: altered pharmacokinetics, increased serum half-life, increase binding affinity, reduced binding affinity, reduced immunogenicity, increased production, altered Fc ligand binding, enhanced or reduced ADCC or CDC activity, altered glycosylation and/or disulfide bonds and modified binding specificity.

### Amino Acid Sequences of Anti-CD3 Portions of Anti-CD3×SLAMF6 Bispecific Antibodies

In some embodiments, the anti-CD3 portion of the anti-CD3×SLAMF6 bispecific antibody is a single-chain variable fragment (scFv) with a fragment crystallizable (Fc) region (i.e., scFv-Fc antibody) comprising a signal peptide, a variable heavy chain (VH) domain, a linker, a variable light chain (VL) domain, and an Fc portion. Underlined and bolded amino acids represent the respective complementarity determining regions (CDRs). Double underlined amino acids represent a linker sequence between the variable heavy chain and the variable light chain portions. Underlined amino acids represent the fragment, crystallizable (Fc) region. Bolded, underlined, and italicized amino acids represent the “hole” mutations for the production of knob-in-hole antibodies. In some embodiments, instead of the “hole” mutations, the amino acid sequence comprising the anti-CD3 portion of an anti-CD3×SLAMF6 bispecific antibody can comprise the “knob” mutations, while the “hole” mutations are present on an anti-SLAMF6 portion of an anti-CD3×SLAMF6 bispecific antibody.

In some embodiments, the amino acid sequence of the anti-CD3 portion (scFv-Fc) of the anti-CD3×SLAMF6 bispecific antibody comprises a signal peptide comprising SEQ ID NO: 1. MGWSCIILFLVATATGVHS (SEQ ID NO: 1). In some embodiments, the amino acid sequence of the anti-CD3 portion (scFv-Fc) of an anti-CD3×SLAMF6 bispecific antibody (without the signal peptide) comprises SEQ ID NO:2.

(SEQ ID NO: 2)
QVQLVQSGAEVKKPGASVKVSCKASGYTFTRYTMHWVRQAPGQGLEWMGYINPSRGY
TNYNQKFKDRVTITADKSTSTAYMELSSLRSEDTAVYYCARYYDDHYCLDYWGQGTTV
TVSSGGGGSGGGGSGGGGSGSEIVLTQSPATLSLSPGERATLSCSASSSVSYMNWYQQKPG
KAPKRLIYDTSKLASGVPSRFSGSGSGTDFTLTINSLEAEDAATYYCQQWSSNPFTFGQGT
KVEIKRLEPKSCDKTHTCPPCPAPEFQGGPSVFLFPPKPKDTLYITREPEVTCVVVDVSHEDP
EVKFNWYVDGVEVHNAKTKPREEQYNSTYRVVSVLTVLHQDWLNGKEYKCQVSNKALP
APIEKTISKAKGQPREPQVYVYPPSRDELTKNQVSLTCLVKGFYPSDIAVEWESNGQPENNY
KTTPPVLDSDGSFALVSKLTVDKSRWQQGNVFSCSVMHEALHNHYTQKSLSLSPG.

In some embodiments, the amino acid sequence of the anti-CD3 portion (scFv-Fc) of an anti-CD3×SLAMF6 bispecific antibody comprises SEQ ID NO: 1 immediately followed by SEQ ID NO: 2. In some embodiments, the signal peptide is cleaved during post-translational modifications that occur in vitro or in vivo.

In some embodiments, the signal peptide of the anti-CD3 portion (scFv-Fc) of the anti-CD3×SLAMF6 bispecific antibody comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-CD3 portion (scFv-Fc) of the anti-CD3×SLAMF6 bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 2. In some embodiments, the anti-CD3 portion of an anti-CD3×SLAMF6 bispecific antibody comprises an amino acid sequence comprising the CDRs of the variable heavy chain domain and the CDRs of the variable light chain domain of the anti-CD3 portion of the anti-CD3×SLAMF6 bispecific antibody wherein the CDR sequences are indicated above (in bold and underline). In some embodiments, the framework regions (FRs) of the variable heavy chain domain and the FRs of the variable light chain domain of the anti-CD3 portion of the anti-CD3×SLAMF6 bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 2.

Although the sequence above is depicted with a linker “GGGGSGGGGSGGGGS” (SEQ ID NO: 14) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker is between about 2 and 25 amino acids in length. In some embodiments, the glycine-serine linker is about 15 amino acids in length. Further discussion of the anti-CD43 portion of the anti-CD3×SLAMF6 bispecific antibodies can be found in Alegre M L et al. Effect of a single amino acid mutation on the activating and immunosuppressive properties of a “humanized” OKT3 monoclonal antibody. J Immunol. 1992 Jun. 1; 148(11):3461-8. PMID: 1534096, incorporated herein by reference in its entirety.

### Nucleic Acid Sequences of Anti-CD3 Portions of Anti-CD3×SLAMF6 Bispecific Antibodies

The amino acid sequences of the respective CDRs, linker portions, VH portion, VL portion, Fc portion, and knob/hole mutations are provided herein. Nucleic acid sequences encoding the respective CDRs, linker portions, VH portion, VL portion, Fc portion and knob/hole mutations can be determined from the amino acid sequence by a person of skill in the art. Nucleic acid sequences encoding the respective CDRs, linker portions, VH portion, VL portion, Fc portion and knob/hole mutations in SEQ ID NO:4 can be determined from the amino acid sequence of SEQ ID NO: 2 by a person of skill in the art. In some embodiments, instead of the “hole” mutations, the nucleic acid sequence comprising the anti-CD3 portion of an anti-CD3×SLAMF6 bispecific antibody can comprise a nucleic acid sequence encoding the “knob” mutations, while the “hole” mutations are present on an anti-SLAMF6 portion of an anti-CD3×SLAMF6 bispecific antibody. In some embodiments, the nucleic acid sequences may be codon optimized.

In some embodiments, the nucleic acid sequence of the anti-CD3 portion (scFv-Fc) of the anti-CD3×SLAMF6 bispecific antibody comprises a nucleic acid sequence encoding a signal peptide comprising SEQ ID NO: 3. ATGGGCTGGAGCTGCATTATCCTGTTCCTGGTGGCCACAGCCACCGGCGTGCACAGC (SEQ ID NO: 3). In some embodiments, the nucleic acid sequence encoding the anti-CD3 portion (scFv-Fc) of an anti-CD3×SLAMF6 bispecific antibody (without the signal peptide) comprises SEQ ID NO: 4. CAGGTGCAGCTGGTGCAGAGCGGAGCAGAGGTGAAGAAGCCAGGAGCCTCTGTGAAG GTGAGCTGCAAGGCCTCCGGCTACACCTTCACACGGTATACCATGCACTGGGTGAGAC AGGCACCTGGACAGGGCCTGGAGTGGATGGGCTACATCAACCCAAGCCGGGGCTACAC AAACTATAATCAGAAGTTTAAGGACAGAGTGACCATCACAGCCGATAAGAGCACCTCC ACAGCCTATATGGAGCTGAGCTCCCTGAGGTCCGAGGACACCGCCGTGTACTATTGCG CCCGCTACTATGACGATCACTACTGTCTGGATTATTGGGGCCAGGGCACCACAGTGAC AGTGTCTAGCGGCGGAGGAGGCTCTGGAGGAGGAGGCAGCGGCGGCGGCGGCTCTGG CAGCGAGATCGTGCTGACCCAGTCCCCAGCCACACTGTCCCTGTCTCCAGGAGAGAGG GCCACCCTGAGCTGCTCCGCCTCCTCTAGCGTGTCTTACATGAATTGGTATCAGCAGAA GCCCGGCAAGGCCCCTAAGAGGCTGATCTACGACACCTCTAAGCTGGCAAGCGGAGTG CCCTCCCGCTTCTCTGGCAGCGGCTCCGGCACCGACTTTACCCTGACAATCAACTCCCT GGAGGCCGAGGATGCCGCCACATACTATTGTCAGCAGTGGTCCTCTAATCCTTTCACCT TTGGCCAGGGCACAAAGGTGGAGATCAAGCGGCTCGAGCCCAAGAGCTGCGACAAGA CCCACACCTGTCCTCCATGTCCTGCTCCAGAGTTTCAAGGCGGCCCTTCCGTGTTCCTGT TTCCTCCAAAGCCTAAGGACACCCTGTACATCACCCGCGAGCCTGAAGTGACCTGTGTG GTGGTGGATGTGTCCCACGAGGACCCCGAAGTGAAGTTCAATTGGTACGTGGACGGCG TGGAAGTGCACAACGCCAAGACCAAGCCTAGAGAGGAACAGTACAACAGCACCTACA GAGTGGTGTCCGTGCTGACCGTGCTGCACCAGGATTGGCTGAACGGCAAAGAGTACAA GTGCCAGGTGTCCAACAAGGCCCTGCCTGCTCCTATCGAGAAAACCATCAGCAAGGCC AAGGGCCAGCCTAGGGAACCTCAAGTGTACGTGTACCCTCCTAGCCGGGACGAGCTGA CCAAGAATCAGGTGTCCCTGACCTGCCTCGTGAAGGGCTTCTACCCTTCCGACATCGCC GTGGAATGGGAGAGCAATGGCCAGCCTGAGAACAACTACAAGACAACCCCTCCTGTGC TGGACAGCGACGGCTCTTTTGCCCTGGTGTCCAAGCTGACAGTGGACAAGTCCAGATG GCAGCAGGGCAACGTGTTCAGCTGCAGCGTGATGCACGAGGCCCTGCACAACCACTAC ACCCAGAAGTCCCTGAGCCTGTCTCCTGGATGA (SEQ ID NO: 4). In some embodiments, the nucleic acid sequence encoding the anti-CD3 portion (scFv-Fc) of an anti-CD3×SLAMF6 bispecific antibody comprises SEQ ID NO: 1 immediately followed by SEQ ID NO: 4. In some embodiments, the signal peptide is cleaved during post-translational modifications that occur in vitro or in vivo.

In some embodiments, the nucleic acid sequence encoding a signal peptide of the anti-CD3 portion (scFv-Fc) of the anti-CD3×SLAMF6 bispecific antibody comprise a nucleic acid sequence 80, 81, 82, 83, 84, 85, 86, 87, 88, 89, 90, 91, 92, 93, 94, 95, 96, 97, 98 or 99% identical to SEQ ID NO: 1. In some embodiments, the nucleic acid sequence encoding an anti-CD3 portion (scFv-Fc) of the anti-CD3×SLAMF6 bispecific antibody (without the signal peptide) comprise a nucleic acid sequence 80, 81, 82, 83, 84, 85, 86, 87, 88, 89, 90, 91, 92, 93, 94, 95, 96, 97, 98 or 99% identical to SEQ ID NO: 4. In some embodiments, the nucleic acid sequence encodes CDRs of the variable heavy chain domain and the CDRs of the variable light chain domain of the anti-CD3 portion (scFv-Fc) of the anti-CD3×SLAMF6 bispecific antibody wherein the CDR sequences are indicated above (in bold and underline). In some embodiments, the nucleic acid sequence encoding framework regions (FRs) of the variable heavy chain domain and the FRs of the variable light chain domain of the anti-CD3 portion of the anti-CD3×SLAMF6 bispecific antibody comprise a nucleic acid sequence 80, 81, 82, 83, 84, 85, 86, 87, 88, 89, 90, 91, 92, 93, 94, 95, 96, 97, 98 or 99% identical to the FRs of SEQ ID NO: 4.

Although the sequence above is depicted with a nucleic acid sequence encoding a linker “GGAGGCGGAGGTTCTGGCGGCGGAGGAAGTGGTGGCGGAGGCTCT” (SEQ ID NO: 15) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker comprises a nucleic acid sequence encoding a linker that is between about 2 and 25 amino acids in length. In some embodiments, the nucleic acid sequence encodes a glycine-serine linker that is about 15 amino acids in length.

### Amino Acid Sequences of Anti-CD45 Portions of Anti-CD45×SLAMF6 Bispecific Antibodies

In some embodiments, the anti-CD45 portion of the anti-CD45×SLAMF6 bispecific antibody is a single-chain variable fragment (scFv) with a fragment crystallizable (Fc) region (i.e., scFv-Fc antibody) comprising a signal peptide, a variable heavy chain (VH) domain, a linker, a variable light chain (VL) domain, and an Fc portion. Underlined and bolded amino acids represent the respective complementarity determining regions (CDRs). Double underlined amino acids represent a linker sequence between the variable heavy chain and the variable light chain portions. Underlined amino acids represent the fragment, crystallizable (Fc) region. Bolded, underlined, and italicized amino acids represent the “hole” mutations for the production of knob-in-hole antibodies. In some embodiments, instead of the “hole” mutations, the amino acid sequence comprising the anti-CD45 portion of an anti-CD45×SLAMF6 bispecific antibody can comprise the “knob” mutations, while the “hole” mutations are present on an anti-SLAMF6 portion of an anti-CD45×SLAMF6 bispecific antibody.

In some embodiments, the amino acid sequence of the anti-CD45 portion (scFv-Fc) of the anti-CD45×SLAMF6 bispecific antibody comprises a signal peptide comprising SEQ ID NO: 1. MGWSCIILFLVATATGVHS (SEQ ID NO: 1). In some embodiments, the amino acid sequence of the anti-CD45 portion (scFv-Fc) of an anti-CD45×SLAMF6 bispecific antibody (without the signal peptide) comprises SEQ ID NO: 5.

(SEQ ID NO: 5)
EVQLVESGGGLVQPGGSLRLSCAASGFTFNNYWMTWVRQAPGKGLEWVASISSSGGSIY
YPDSVKGRFTISRDNSKNTLYLQMNSLRAEDTAVYYCARDERWAGAMDAWGQGTTVTV
SSGGGGSGGGGSGGGGSDIQMTQSPSSLSASVGDRVTITCKASQNINKNLDWYQQKPGKA
PKLLIYETNNLQTGVPSRFSGSGSGTDFTLTISSLQPEDFATYYCYQHNSRFTFGGGTKLEI
KRLEPKSCDKTHTCPPCPAPEFQGGPSVFLFPPKPKDTLYITREPEVTCVVVDVSHEDPEVK
FNWYVDGVEVHNAKTKPREEQYNSTYRVVSVLTVLHQDWLNGKEYKCQVSNKALPAPIE
KTISKAKGQPREPQVYVYPPSRDELTKNQVSLTCLVKGFYPSDIAVEWESNGQPENNYKTT
PPVLDSDGSFALVSKLTVDKSRWQQGNVFSCSVMHEALHNHYTQKSLSLSPG.

In some embodiments, the amino acid sequence of the anti-CD45 portion (scFv-Fc) of an anti-CD45×SLAMF6 bispecific antibody comprises SEQ ID NO: 1 immediately followed by SEQ ID NO: 5. In some embodiments, the signal peptide is cleaved during post-translational modifications that occur in vitro or in vivo.

In some embodiments, the amino acid sequence of the anti-CD45 portion (scFv-Fc) of the anti-CD45×SLAMF6 bispecific antibody comprises a signal peptide comprising SEQ ID NO: 1. MGWSCIILFLVATATGVHS (SEQ ID NO: 1). In some embodiments, the amino acid sequence of the anti-CD45 portion (scFv-Fc) of an anti-CD45×SLAMF6 bispecific antibody (without the signal peptide) comprises SEQ ID NO: 28.

(SEQ ID NO: 28)
EVQLQQSGPELEKPGASVKISCKASGYSFTGYNMNWVKQSNGKSLEWIGNIDPYYGGSD
YSQKFLGKATLTVDKSSSTAYMHLKSLTSEDSAVYYCARSNMYDGEYYVMDYWGQGTS
VTVSSGGGGSGGGGSGGGGSDIVLTQSPASLAVSLGQRATISCRASESVDNFGVSFMNWF
QQKPGQPPKLLIYASSNRGSGVPARFSGSGSGTDFSLNVHPMEEDDAAMYFCQQSKAVPR
TFGGGTKLEIKPKSCDKTHTCPPCPAPEFQGGPSVFLFPPKPKDTLYITREPEVTCVVVDVSH
EDPEVKFNWYVDGVEVHNAKTKPREEQYNSTYRVVSVLTVLHQDWLNGKEYKCQVSNK
ALPAPIEKTISKAKGQPREPQVYVYPPSRDELTKNQVSLTCLVKGFYPSDIAVEWESNGQPE
NNYKTTPPVLDSDGSFALVSKLTVDKSRWQQGNVFSCSVMHEALHNHYTQKSLSLSPG.

In some embodiments, the amino acid sequence of the anti-CD45 portion (scFv-Fc) of an anti-CD45×SLAMF6 bispecific antibody comprises SEQ ID NO: 1 immediately followed by SEQ ID NO: 28. In some embodiments, the signal peptide is cleaved during post-translational modifications that occur in vitro or in vivo.

In some embodiments, the amino acid sequence of the anti-CD45 portion (scFv-Fc) of an anti-CD45×SLAMF6 bispecific antibody (without the signal peptide) comprises SEQ ID NO: 29.

(SEQ ID NO: 29)
DVQLVESGGGLVQPGGSRKLSCAASGFTFSNFGMHWVRQAPEKGLEWVAYVSRGSTTF
YYADTVKGRFTISRDNPKNTLFLQMTSLRSEDTAMYYCARSATATWTMDYWGHGTSVT
VSSGGGGSGGGGSGGGGSNIMMTQSPSSLAVSAGEKVTMSCKSSQSVFSSSNHMNYLAW
YQQKPGQSPKLLIYWASTRESGVPDRFTGSGSGTDFTLTISSVQAEDLAVYYCHQYLSSW
TFGGGSKLEIKPKSCDKTHTCPPCPAPEFQGGPSVFLFPPKPKDTLYITREPEVTCVVVDVSH
EDPEVKFNWYVDGVEVHNAKTKPREEQYNSTYRVVSVLTVLHQDWLNGKEYKCQVSNK
ALPAPIEKTISKAKGQPREPQVYVYPPSRDELTKNQVSLTCLVKGFYPSDIAVEWESNGQPE
NNYKTTPPVLDSDGSFALVSKLTVDKSRWQQGNVFSCSVMHEALHNHYTQKSLSLSPG.

In some embodiments, the amino acid sequence of the anti-CD45 portion (scFv-Fc) of an anti-CD45×SLAMF6 bispecific antibody comprises SEQ ID NO: 1 immediately followed by SEQ ID NO: 29. In some embodiments, the signal peptide is cleaved during post-translational modifications that occur in vitro or in vivo.

In some embodiments, the amino acid sequence of the anti-CD45 portion (scFv-Fc) of an anti-CD45×SLAMF6 bispecific antibody (without the signal peptide) comprises SEQ ID NO: 30.

(SEQ ID NO: 30)
QVQLQQSAPELARPGASVRMSCKASGYTFTTYTMNWLTQRPGQGLEWIGYINPSSGYTE
YNQKFKDKTSLTADTSSSTAYMQLSSLTSEHSAVYYCARASGYSSWFAYWGQGTLVTVS
AGGGGSGGGGSGGGGSNIVMTQTPKFLLVSAGDRVTITCKASQSVNNDVGWYQQKPGQS
PKLLIYYASNRYTGVPDRFTGSGYGTDFTFTISTVQAEDLAVYFCQQDYNSPLTFGAGTKL
ELKPKSCDKTHTCPPCPAPEFQGGPSVFLFPPKPKDTLYITREPEVTCVVVDVSHEDPEVKF
NWYVDGVEVHNAKTKPREEQYNSTYRVVSVLTVLHQDWLNGKEYKCQVSNKALPAPIEK
TISKAKGQPREPQVYVYPPSRDELTKNQVSLTCLVKGFYPSDIAVEWESNGQPENNYKTTPP
VLDSDGSFALVSKLTVDKSRWQQGNVFSCSVMHEALHNHYTQKSLSLSPG.

In some embodiments, the amino acid sequence of the anti-CD45 portion (scFv-Fc) of an anti-CD45×SLAMF6 bispecific antibody comprises SEQ ID NO: 1 immediately followed by SEQ ID NO: 30. In some embodiments, the signal peptide is cleaved during post-translational modifications that occur in vitro or in vivo.

In some embodiments, the amino acid sequence of the anti-CD45 portion (scFv-Fc) of an anti-CD45×SLAMF6 bispecific antibody (without the signal peptide) comprises SEQ ID NO: 31.

(SEQ ID NO: 31)
QVQLQQSGAELARPGASVKMSCKASGYTFTFYAILWVKQMPGQGLEWIGFINPSSGYTSY
NQKFKDKATLTADKSSSTAYMQLSSLTSEDSAVYYCARDYGLDYWGQGTTLAVSSGGGG
SGGGGSGGGGSDIVMTQAAPSVPVTPGESVSISCRSSKSLLHSNGNTYLYWFLQRPGQSPQ
LLIYRMSNLASGVPDRFSGSGSGTAFTLGISRVEAGDVGVYYCMQHLEYPFTFGSGTKLEI
KPKSCDKTHTCPPCPAPEFQGGPSVFLFPPKPKDTLYITREPEVTCVVVDVSHEDPEVKFNW
YVDGVEVHNAKTKPREEQYNSTYRVVSVLTVLHQDWLNGKEYKCQVSNKALPAPIEKTIS
KAKGQPREPQVYVYPPSRDELTKNQVSLTCLVKGFYPSDIAVEWESNGQPENNYKTTPPVL
DSDGSFALVSKLTVDKSRWQQGNVFSCSVMHEALHNHYTQKSLSLSPG.

In some embodiments, the amino acid sequence of the anti-CD45 portion (scFv-Fc) of an anti-CD45×SLAMF6 bispecific antibody comprises SEQ ID NO: 1 immediately followed by SEQ ID NO: 31. In some embodiments, the signal peptide is cleaved during post-translational modifications that occur in vitro or in vivo.

In some embodiments, the amino acid sequence of the anti-CD45 portion (scFv-Fc) of an anti-CD45×SLAMF6 bispecific antibody (without the signal peptide) comprises SEQ ID NO: 32.

(SEQ ID NO: 32)
DVQLVESGGGLVQPGGSRKLSCAASGFTFSSFGMHWVRQAPEKGLEWVA
YINSGSTTFYYADTVKGRFTISRDNPKNTLFLQMTSLGSEDTAMYYCAR
SATATWTMDYWGQGTSVTVSSGGGGSGGGGSGGGGSNIMMTQSPSSLAV
SAGEKVTMSCKSSQSVFVSSNQKNYLAWYQQKPGQSPKLLIYWASTRES
AVPDRFTGSGSGTDFTLTIGSVQAEDLAVYYCHQYLSSWTFGGGTKLEI
KPKSCDKTHTCPPCPAPEFQGGPSVFLFPPKPKDTLYITREPEVTCVVV
DVSHEDPEVKFNWYVDGVEVHNAKTKPREEQYNSTYRVVSVLTVLHQDW
LNGKEYKCQVSNKALPAPIEKTISKAKGQPREPQVYVYPPSRDELTKNQ
VSLTCLVKGFYPSDIAVEWESNGQPENNYKTTPPVLDSDGSFALVSKLT
VDKSRWQQGNVFSCSVMHEALHNHYTQKSLSLSPG.

In some embodiments, the amino acid sequence of the anti-CD45 portion (scFv-Fc) of an anti-CD45×SLAMF6 bispecific antibody comprises SEQ ID NO: 1 immediately followed by SEQ ID NO: 32. In some embodiments, the signal peptide is cleaved during post-translational modifications that occur in vitro or in vivo.

In some embodiments, the amino acid sequence of the anti-CD45 portion (scFv-Fc) of an anti-CD45×SLAMF6 bispecific antibody (without the signal peptide) comprises SEQ ID NO: 33.

(SEQ ID NO: 33)
EVQLQQSGAELVKPGASVKLSCTASGFNIKDTFMHWVKLRPEQGLEWIG
RIDPANGYTKYDPRFQGKATIIADTSSNTAYLQLSSLTSEDTAVYYCAS
GEYYALDYWGQGTSVTVSSGGGGSGGGGSGGGGSDIVLTQSPASLTVSL
GQRATISCRASKSVSTSGYSYMHWYQQKPGQPPKLLIYLASNLESGVPA
RFSGSGSGTDFTLNIHPVEEEDAATYYCQHNRELPYTFGGGTKLEIKPK
SCDKTHTCPPCPAPEFQGGPSVFLFPPKPKDTLYITREPEVTCVVVDVS
HEDPEVKFNWYVDGVEVHNAKTKPREEQYNSTYRVVSVLTVLHQDWLNG
KEYKCQVSNKALPAPIEKTISKAKGQPREPQVYVYPPSRDELTKNQVSL
TCLVKGFYPSDIAVEWESNGQPENNYKTTPPVLDSDGSFALVSKLTVDK
SRWQQGNVFSCSVMHEALHNHYTQKSLSLSPG.

In some embodiments, the amino acid sequence of the anti-CD45 portion (scFv-Fc) of an anti-CD45×SLAMF6 bispecific antibody comprises SEQ ID NO: 1 immediately followed by SEQ ID NO: 33. In some embodiments, the signal peptide is cleaved during post-translational modifications that occur in vitro or in vivo.

In some embodiments, the signal peptide of the anti-CD45 portion (scFv-Fc) of the anti-CD45×SLAMF6 bispecific antibody comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-CD45 portion (scFv-Fc) of the anti-CD45×SLAMF6 bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 5. In some embodiments, the anti-CD45 portion (scFv-Fc) of the anti-CD45×SLAMF6 bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 28. In some embodiments, the anti-CD45 portion (scFv-Fc) of the anti-CD45×SLAMF6 bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 29. In some embodiments, the anti-CD45 portion (scFv-Fc) of the anti-CD45×SLAMF6 bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 30. In some embodiments, the anti-CD45 portion (scFv-Fc) of the anti-CD45×SLAMF6 bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 31. In some embodiments, the anti-CD45 portion (scFv-Fc) of the anti-CD45×SLAMF6 bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 32. In some embodiments, the anti-CD45 portion (scFv-Fc) of the anti-CD45×SLAMF6 bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 33. In some embodiments, the anti-CD45 portion of an anti-CD45×SLAMF6 bispecific antibody comprises an amino acid sequence comprising the CDRs of the variable heavy chain domain and the CDRs of the variable light chain domain of the anti-CD45 portion of the anti-CD45×SLAMF6 bispecific antibody wherein the CDR sequences are indicated above (in bold and underline). In some embodiments, the framework regions (FRs) of the variable heavy chain domain and the FRs of the variable light chain domain of the anti-CD45 portion of the anti-CD45×SLAMF6 bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 5. In some embodiments, the framework regions (FRs) of the variable heavy chain domain and the FRs of the variable light chain domain of the anti-CD45 portion of the anti-CD45×SLAMF6 bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 28. In some embodiments, the framework regions (FRs) of the variable heavy chain domain and the FRs of the variable light chain domain of the anti-CD45 portion of the anti-CD45×SLAMF6 bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 29. In some embodiments, the framework regions (FRs) of the variable heavy chain domain and the FRs of the variable light chain domain of the anti-CD45 portion of the anti-CD45×SLAMF6 bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 30. In some embodiments, the framework regions (FRs) of the variable heavy chain domain and the FRs of the variable light chain domain of the anti-CD45 portion of the anti-CD45×SLAMF6 bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 31. In some embodiments, the framework regions (FRs) of the variable heavy chain domain and the FRs of the variable light chain domain of the anti-CD45 portion of the anti-CD45×SLAMF6 bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 32. In some embodiments, the framework regions (FRs) of the variable heavy chain domain and the FRs of the variable light chain domain of the anti-CD45 portion of the anti-CD45×SLAMF6 bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 33.

Although the sequence above is depicted with a linker “GGGGSGGGGSGGGGS” (SEQ ID NO: 14) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker is between about 2 and 25 amino acids in length. In some embodiments, the glycine-serine linker is about 15 amino acids in length.

### Nucleic Acid Sequences of Anti-CD45 Portions of Anti-CD45×SLAMF6 Bispecific Antibodies

The amino acid sequences of the respective CDRs, linker portions, VH portion, VL portion, Fc portion, and knob/hole mutations are provided herein. Nucleic acid sequences encoding the respective CDRs, linker portions, VH portion, VL portion, Fc portion and knob/hole mutations can be determined from the amino acid sequence by a person of skill in the art. Nucleic acid sequences encoding the respective CDRs, linker portions, VH portion, VL portion, Fc portion and knob/hole mutations in SEQ ID NO:6 can be determined from the amino acid sequence of SEQ ID NO: 5 by a person of skill in the art. In some embodiments, instead of the “hole” mutations, the nucleic acid sequence comprising the anti-CD45 portion of an anti-CD45×SLAMF6 bispecific antibody can comprise a nucleic acid sequence encoding the “knob” mutations, while the “hole” mutations are present on an anti-SLAMF6 portion of an anti-CD45×SLAMF6 bispecific antibody. In some embodiments, the nucleic acid sequences may be codon optimized.

In some embodiments, the nucleic acid sequence of the anti-CD45 portion (scFv-Fc) of the anti-CD45×SLAMF6 bispecific antibody comprises a nucleic acid sequence encoding a signal peptide comprising SEQ ID NO: 3. ATGGGCTGGAGCTGCATTATCCTGTTCCTGGTGGCCACAGCCACCGGCGTGCACAGC (SEQ ID NO: 3). In some embodiments, the nucleic acid sequence encoding the anti-CD45 portion (scFv-Fc) of an anti-CD45×SLAMF6 bispecific antibody (without the signal peptide) comprises SEQ ID NO: 6. GAGGTGCAGCTGGTTGAATCTGGCGGAGGACTGGTTCAGCCTGGCGGATCTCTGAGAC TGTCTTGTGCCGCCAGCGGCTTCACCTTCAACAACTACTGGATGACCTGGGTCCGACAG GCCCCTGGCAAAGGACTTGAATGGGTCGCCAGCATCTCTAGCAGCGGCGGCAGCATCT ACTACCCCGATTCTGTGAAGGGCAGATTCACCATCAGCCGGGACAACAGCAAGAACAC CCTGTACCTGCAGATGAACAGCCTGAGAGCCGAGGACACCGCCGTGTACTACTGTGCC AGAGATGAGAGATGGGCTGGCGCCATGGATGCTTGGGGACAGGGAACAACCGTGACC GTTTCTTCTGGCGGCGGAGGAAGCGGAGGCGGAGGCTCCGGTGGTGGTGGATCTGACA TCCAGATGACACAGAGCCCCAGCAGCCTGTCTGCCTCTGTGGGAGACAGAGTGACCAT CACATGCAAGGCCAGCCAGAACATCAACAAGAACCTGGATTGGTATCAGCAGAAGCCC GGCAAGGCCCCTAAGCTGCTGATCTACGAGACAAACAACCTGCAGACCGGCGTGCCCA GCAGATTTTCTGGCTCTGGCAGCGGCACCGACTTCACCCTGACCATATCTAGCCTGCAG CCTGAGGACTTCGCCACCTACTACTGCTACCAGCACAACAGCCGGTTCACCTTTGGCGG AGGCACCAAGCTGGAAATCAAGCGGCTCGAGCCCAAGAGCTGCGACAAGACCCACAC CTGTCCTCCATGTCCTGCTCCAGAGTTTCAAGGCGGCCCTTCCGTGTTCCTGTTTCCTCC AAAGCCTAAGGACACCCTGTACATCACCCGCGAGCCTGAAGTGACCTGTGTGGTGGTG GATGTGTCCCACGAGGACCCCGAAGTGAAGTTCAATTGGTACGTGGACGGCGTGGAAG TGCACAACGCCAAGACCAAGCCTAGAGAGGAACAGTACAACAGCACCTACAGAGTGG TGTCCGTGCTGACCGTGCTGCACCAGGATTGGCTGAACGGCAAAGAGTACAAGTGCCA GGTGTCCAACAAGGCCCTGCCTGCTCCTATCGAGAAAACCATCAGCAAGGCCAAGGGC CAGCCTAGGGAACCTCAAGTGTACGTGTACCCTCCTAGCCGGGACGAGCTGACCAAGA ATCAGGTGTCCCTGACCTGCCTCGTGAAGGGCTTCTACCCTTCCGACATCGCCGTGGAA TGGGAGAGCAATGGCCAGCCTGAGAACAACTACAAGACAACCCCTCCTGTGCTGGACA GCGACGGCTCTTTTGCCCTGGTGTCCAAGCTGACAGTGGACAAGTCCAGATGGCAGCA GGGCAACGTGTTCAGCTGCAGCGTGATGCACGAGGCCCTGCACAACCACTACACCCAG AAGTCCCTGAGCCTGTCTCCTGGATGA (SEQ ID NO: 6). In some embodiments, the nucleic acid sequence encoding the anti-CD45 portion (scFv-Fc) of an anti-CD45×SLAMF6 bispecific antibody comprises SEQ ID NO: 3 immediately followed by SEQ ID NO: 6. In some embodiments, the signal peptide is cleaved during post-translational modifications that occur in vitro or in vivo.

In some embodiments, the nucleic acid sequence of the anti-CD45 portion (scFv-Fc) of the anti-CD45×SLAMF6 bispecific antibody comprises a nucleic acid sequence encoding a signal peptide comprising SEQ ID NO: 3. ATGGGCTGGAGCTGCATTATCCTGTTCCTGGTGGCCACAGCCACCGGCGTGCACAGC (SEQ ID NO: 3). In some embodiments, the nucleic acid sequence encoding the anti-CD45 portion (scFv-Fc) of an anti-CD45×SLAMF6 bispecific antibody (without the signal peptide) comprises SEQ ID NO: 34.

(SEQ ID NO: 34)
GAAGTGCAGCTGCAGCAGAGCGGCCCGGAACTGGAAAAACCGGGCGCGA
GCGTGAAAATTAGCTGCAAAGCGAGCGGCTATAGCTTTACCGGCTATAA
CATGAACTGGGTGAAACAGAGCAACGGCAAAAGCCTGGAATGGATTGGC
AACATTGATCCGTATTATGGCGGCAGCGATTATAGCCAGAAATTTCTGG
GCAAAGCGACCCTGACCGTGGATAAAAGCAGCAGCACCGCGTATATGCA
TCTGAAAAGCCTGACCAGCGAAGATAGCGCGGTGTATTATTGCGCGCGC
AGCAACATGTATGATGGCGAATATTATGTGATGGATTATTGGGGCCAGG
GCACCAGCGTGACCGTGAGCAGCGGCGGCGGCGGCAGCGGCGGCGGCGG
CAGCGGCGGCGGCGGCAGCGATATTGTGCTGACCCAGAGCCCGGCGAGC
CTGGCGGTGAGCCTGGGCCAGCGCGCGACCATTAGCTGCCGCGCGAGCG
AAAGCGTGGATAACTTTGGCGTGAGCTTTATGAACTGGTTTCAGCAGAA
ACCGGGCCAGCCGCCGAAACTGCTGATTTATGCGAGCAGCAACCGCGGC
AGCGGCGTGCCGGCGCGCTTTAGCGGCAGCGGCAGCGGCACCGATTTTA
GCCTGAACGTGCATCCGATGGAAGAAGATGATGCGGCGATGTATTTTTG
CCAGCAGAGCAAAGCGGTGCCGCGCACCTTTGGCGGCGGCACCAAACTG
GAAATTAAACCGAAAAGCTGCGATAAAACCCATACCTGCCCGCCGTGCC
CGGCGCCGGAATTTCAGGGCGGCCCGAGCGTGTTTCTGTTTCCGCCGAA
ACCGAAAGATACCCTGTATATTACCCGCGAACCGGAAGTGACCTGCGTG
GTGGTGGATGTGAGCCATGAAGATCCGGAAGTGAAATTTAACTGGTATG
TGGATGGCGTGGAAGTGCATAACGCGAAAACCAAACCGCGCGAAGAACA
GTATAACAGCACCTATCGCGTGGTGAGCGTGCTGACCGTGCTGCATCAG
GATTGGCTGAACGGCAAAGAATATAAATGCCAGGTGAGCAACAAAGCGC
TGCCGGCGCCGATTGAAAAAACCATTAGCAAAGCGAAAGGCCAGCCGCG
CGAACCGCAGGTGTATGTGTATCCGCCGAGCCGCGATGAACTGACCAAA
AACCAGGTGAGCCTGACCTGCCTGGTGAAAGGCTTTTATCCGAGCGATA
TTGCGGTGGAATGGGAAAGCAACGGCCAGCCGGAAAACAACTATAAAAC
CACCCCGCCGGTGCTGGATAGCGATGGCAGCTTTGCGCTGGTGAGCAAA
CTGACCGTGGATAAAAGCCGCTGGCAGCAGGGCAACGTGTTTAGCTGCA
GCGTGATGCATGAAGCGCTGCATAACCATTATACCCAGAAAAGCCTGAG
CCTGAGCCCGGGC.

In some embodiments, the nucleic acid sequence encoding the anti-CD45 portion (scFv-Fc) of an anti-CD45×SLAMF6 bispecific antibody comprises SEQ ID NO: 3 immediately followed by SEQ ID NO: 34. In some embodiments, the signal peptide is cleaved during post-translational modifications that occur in vitro or in vivo.

In some embodiments, the nucleic acid sequence of the anti-CD45 portion (scFv-Fc) of the anti-CD45×SLAMF6 bispecific antibody comprises a nucleic acid sequence encoding a signal peptide comprising SEQ ID NO: 3. ATGGGCTGGAGCTGCATTATCCTGTTCCTGGTGGCCACAGCCACCGGCGTGCACAGC (SEQ ID NO: 3). In some embodiments, the nucleic acid sequence encoding the anti-CD45 portion (scFv-Fc) of an anti-CD45×SLAMF6 bispecific antibody (without the signal peptide) comprises SEQ ID NO: 35. GATGTGCAGCTGGTGGAAAGCGGCGGCGGCCTGGTGCAGCCGGGCGGCAGCCGCAAA CTGAGCTGCGCGGCGAGCGGCTTTACCTTTAGCAACTTTGGCATGCATTGGGTGCGCCA GGCGCCGGAAAAAGGCCTGGAATGGGTGGCGTATGTGAGCCGCGGCAGCACCACCTTT TATTATGCGGATACCGTGAAAGGCCGCTTTACCATTAGCCGCGATAACCCGAAAAACA CCCTGTTTCTGCAGATGACCAGCCTGCGCAGCGAAGATACCGCGATGTATTATTGCGCG CGCAGCGCGACCGCGACCTGGACCATGGATTATTGGGGCCATGGCACCAGCGTGACCG TGAGCAGCGGCGGCGGCGGCAGCGGCGGCGGCGGCAGCGGCGGCGGCGGCAGCAACA TTATGATGACCCAGAGCCCGAGCAGCCTGGCGGTGAGCGCGGGCGAAAAAGTGACCAT GAGCTGCAAAAGCAGCCAGAGCGTGTTTAGCAGCAGCAACCATATGAACTATCTGGCG TGGTATCAGCAGAAACCGGGCCAGAGCCCGAAACTGCTGATTTATTGGGCGAGCACCC GCGAAAGCGGCGTGCCGGATCGCTTTACCGGCAGCGGCAGCGGCACCGATTTTACCCT GACCATTAGCAGCGTGCAGGCGGAAGATCTGGCGGTGTATTATTGCCATCAGTATCTG AGCAGCTGGACCTTTGGCGGCGGCAGCAAACTGGAAATTAAACCGAAAAGCTGCGATA AAACCCATACCTGCCCGCCGTGCCCG GCGCCGGAATTTCAGGGCGGCCCGAGCGTGTTTCTGTTTCCGCCGAAACCGAAAGATA CCCTGTATATTACCCGCGAACCGGAAGTGACCTGCGTGGTGGTGGATGTGAGCCATGA AGATCCGGAAGTGAAATTTAACTGGTATGTGGATGGCGTGGAAGTGCATAACGCGAAA ACCAAACCGCGCGAAGAACAGTATAACAGCACCTATCGCGTGGTGAGCGTGCTGACCG TGCTGCATCAGGATTGGCTGAACGGCAAAGAATATAAATGCCAGGTGAGCAACAAAGC GCTGCCGGCGCCGATTGAAAAAACCATTAGCAAAGCGAAAGGCCAGCCGCGCGAACC GCAGGTGTATGTGTATCCGCCGAGCCGCGATGAACTGACCAAAAACCAGGTGAGCCTG ACCTGCCTGGTGAAAGGCTTTTATCCGAGCGATATTGCGGTGGAATGGGAAAGCAACG GCCAGCCGGAAAACAACTATAAAACCACCCCGCCGGTGCTGGATAGCGATGGCAGCTT TGCGCTGGTGAGCAAACTGACCGTGGATAAAAGCCGCTGGCAGCAGGGCAACGTGTTT AGCTGCAGCGTGATGCATGAAGCGCTGCATAACCATTATACCCAGAAAAGCCTGAGCC TGAGCCCGGGC (SEQ ID NO: 35). In some embodiments, the nucleic acid sequence encoding the anti-CD45 portion (scFv-Fc) of an anti-CD45×SLAMF6 bispecific antibody comprises SEQ ID NO: 3 immediately followed by SEQ ID NO: 35. In some embodiments, the signal peptide is cleaved during post-translational modifications that occur in vitro or in vivo.

In some embodiments, the nucleic acid sequence of the anti-CD45 portion (scFv-Fc) of the anti-CD45×SLAMF6 bispecific antibody comprises a nucleic acid sequence encoding a signal peptide comprising SEQ ID NO: 3. In some embodiments, the nucleic acid sequence encoding the anti-CD45 portion (scFv-Fc) of an anti-CD45×SLAMF6 bispecific antibody (without the signal peptide) comprises SEQ ID NO: 36. CAGGTGCAGCTGCAGCAGAGCGGCGCGGAACTGGCGCGCCCGGGCGCGAGCGTGAAA ATGAGCTGCAAAGCGAGCGGCTATACCTTTACCTTTTATGCGATTCTGTGGGTGAAACA GATGCCGGGCCAGGGCCTGGAATGGATTGGCTTTATTAACCCGAGCAGCGGCTATACC AGCTATAACCAGAAATTTAAAGATAAAGCGACCCTGACCGCGGATAAAAGCAGCAGC ACCGCGTATATGCAGCTGAGCAGCCTGACCAGCGAAGATAGCGCGGTGTATTATTGCG CGCGCGATTATGGCCTGGATTATTGGGGCCAGGGCACCACCCTGGCGGTGAGCAGCGG CGGCGGCGGCAGCGGCGGCGGCGGCAGCGGCGGCGGCGGCAGCGATATTGTGATGAC CCAGGCGGCGCCGAGCGTGCCGGTGACCCCGGGCGAAAGCGTGAGCATTAGCTGCCGC AGCAGCAAAAGCCTGCTGCATAGCAACGGCAACACCTATCTGTATTGGTTTCTGCAGC GCCCGGGCCAGAGCCCGCAGCTGCTGATTTATCGCATGAGCAACCTGGCGAGCGGCGT GCCGGATCGCTTTAGCGGCAGCGGCAGCGGCACCGCGTTTACCCTGGGCATTAGCCGC GTGGAAGCGGGCGATGTGGGCGTGTATTATTGCATGCAGCATCTGGAATATCCGTTTAC CTTTGGCAGCGGCACCAAACTGGAAATTAAACCGAAAAGCTGCGATAAAACCCATACC TGCCCGCCGTGCCCGGCGCCGGAATTTCAGGGCGGCCCGAGCGTGTTTCTGTTTCCGCC GAAACCGAAAGATACCCTGTATATTACCCGCGAACCGGAAGTGACCTGCGTGGTGGTG GATGTGAGCCATGAAGATCCGGAAGTGAAATTTAACTGGTATGTGGATGGCGTGGAAG TGCATAACGCGAAAACCAAACCGCGCGAAGAACAGTATAACAGCACCTATCGCGTGGT GAGCGTGCTGACCGTGCTGCATCAGGATTGGCTGAACGGCAAAGAATATAAATGCCAG GTGAGCAACAAAGCGCTGCCGGCGCCGATTGAAAAAACCATTAGCAAAGCGAAAGGC CAGCCGCGCGAACCGCAGGTGTATGTGTATCCGCCGAGCCGCGATGAACTGACCAAAA ACCAGGTGAGCCTGACCTGCCTGGTGAAAGGCTTTTATCCGAGCGATATTGCGGTGGA ATGGGAAAGCAACGGCCAGCCGGAAAACAACTATAAAACCACCCCGCCGGTGCTGGA TAGCGATGGCAGCTTTGCGCTGGTGAGCAAACTGACCGTGGATAAAAGCCGCTGGCAG CAGGGCAACGTGTTTAGCTGCAGCGTGATGCATGAAGCGCTGCATAACCATTATACCC AGAAAAGCCTGAGCCTGAGCCCGGGC (SEQ ID NO: 36). In some embodiments, the nucleic acid sequence encoding the anti-CD45 portion (scFv-Fc) of an anti-CD45×SLAMF6 bispecific antibody comprises SEQ ID NO: 3 immediately followed by SEQ ID NO: 36. In some embodiments, the signal peptide is cleaved during post-translational modifications that occur in vitro or in vivo.

In some embodiments, the nucleic acid sequence of the anti-CD45 portion (scFv-Fc) of the anti-CD45×SLAMF6 bispecific antibody comprises a nucleic acid sequence encoding a signal peptide comprising SEQ ID NO: 3. In some embodiments, the nucleic acid sequence encoding the anti-CD45 portion (scFv-Fc) of an anti-CD45×SLAMF6 bispecific antibody (without the signal peptide) comprises SEQ ID NO: 37. CAGGTGCAGCTGCAGCAGAGCGGCGCGGAACTGGCGCGCCCGGGCGCGAGCGTGAAA ATGAGCTGCAAAGCGAGCGGCTATACCTTTACCTTTTATGCGATTCTGTGGGTGAAACA GATGCCGGGCCAGGGCCTGGAATGGATTGGCTTTATTAACCCGAGCAGCGGCTATACC AGCTATAACCAGAAATTTAAAGATAAAGCGACCCTGACCGCGGATAAAAGCAGCAGC ACCGCGTATATGCAGCTGAGCAGCCTGACCAGCGAAGATAGCGCGGTGTATTATTGCG CGCGCGATTATGGCCTGGATTATTGGGGCCAGGGCACCACCCTGGCGGTGAGCAGCGG CGGCGGCGGCAGCGGCGGCGGCGGCAGCGGCGGCGGCGGCAGCGATATTGTGATGAC CCAGGCGGCGCCGAGCGTGCCGGTGACCCCGGGCGAAAGCGTGAGCATTAGCTGCCGC AGCAGCAAAAGCCTGCTGCATAGCAACGGCAACACCTATCTGTATTGGTTTCTGCAGC GCCCGGGCCAGAGCCCGCAGCTGCTGATTTATCGCATGAGCAACCTGGCGAGCGGCGT GCCGGATCGCTTTAGCGGCAGCGGCAGCGGCACCGCGTTTACCCTGGGCATTAGCCGC GTGGAAGCGGGCGATGTGGGCGTGTATTATTGCATGCAGCATCTGGAATATCCGTTTAC CTTTGGCAGCGGCACCAAACTGGAAATTAAACCGAAAAGCTGCGATAAAACCCATACC TGCCCGCCGTGCCCGGCGCCGGAATTTCAGGGCGGCCCGAGCGTGTTTCTGTTTCCGCC GAAACCGAAAGATACCCTGTATATTACCCGCGAACCGGAAGTGACCTGCGTGGTGGTG GATGTGAGCCATGAAGATCCGGAAGTGAAATTTAACTGGTATGTGGATGGCGTGGAAG TGCATAACGCGAAAACCAAACCGCGCGAAGAACAGTATAACAGCACCTATCGCGTGGT GAGCGTGCTGACCGTGCTGCATCAGGATTGGCTGAACGGCAAAGAATATAAATGCCAG GTGAGCAACAAAGCGCTGCCGGCGCCGATTGAAAAAACCATTAGCAAAGCGAAAGGC CAGCCGCGCGAACCGCAGGTGTATGTGTATCCGCCGAGCCGCGATGAACTGACCAAAA ACCAGGTGAGCCTGACCTGCCTGGTGAAAGGCTTTTATCCGAGCGATATTGCGGTGGA ATGGGAAAGCAACGGCCAGCCGGAAAACAACTATAAAACCACCCCGCCGGTGCTGGA TAGCGATGGCAGCTTTGCGCTGGTGAGCAAACTGACCGTGGATAAAAGCCGCTGGCAG CAGGGCAACGTGTTTAGCTGCAGCGTGATGCATGAAGCGCTGCATAACCATTATACCC AGAAAAGCCTGAGCCTGAGCCCGGGC (SEQ ID NO: 37). In some embodiments, the nucleic acid sequence encoding the anti-CD45 portion (scFv-Fc) of an anti-CD45×SLAMF6 bispecific antibody comprises SEQ ID NO: 3 immediately followed by SEQ ID NO: 37. In some embodiments, the signal peptide is cleaved during post-translational modifications that occur in vitro or in vivo.

In some embodiments, the nucleic acid sequence of the anti-CD45 portion (scFv-Fc) of the anti-CD45×SLAMF6 bispecific antibody comprises a nucleic acid sequence encoding a signal peptide comprising SEQ ID NO: 3. In some embodiments, the nucleic acid sequence encoding the anti-CD45 portion (scFv-Fc) of an anti-CD45×SLAMF6 bispecific antibody (without the signal peptide) comprises SEQ ID NO: 38. GATGTGCAGCTGGTGGAAAGCGGCGGCGGCCTGGTGCAGCCGGGCGGCAGCCGCAAA CTGAGCTGCGCGGCGAGCGGCTTTACCTTTAGCAGCTTTGGCATGCATTGGGTGCGCCA GGCGCCGGAAAAAGGCCTGGAATGGGTGGCGTATATTAACAGCGGCAGCACCACCTTT TATTATGCGGATACCGTGAAAGGCCGCTTTACCATTAGCCGCGATAACCCGAAAAACA CCCTGTTTCTGCAGATGACCAGCCTGGGCAGCGAAGATACCGCGATGTATTATTGCGCG CGCAGCGCGACCGCGACCTGGACCATGGATTATTGGGGCCAGGGCACCAGCGTGACCG TGAGCAGCGGCGGCGGCGGCAGCGGCGGCGGCGGCAGCGGCGGCGGCGGCAGCAACA TTATGATGACCCAGAGCCCGAGCAGCCTGGCGGTGAGCGCGGGCGAAAAAGTGACCAT GAGCTGCAAAAGCAGCCAGAGCGTGTTTGTGAGCAGCAACCAGAAAAACTATCTGGCG TGGTATCAGCAGAAACCGGGCCAGAGCCCGAAACTGCTGATTTATTGGGCGAGCACCC GCGAAAGCGCGGTGCCGGATCGCTTTACCGGCAGCGGCAGCGGCACCGATTTTACCCT GACCATTGGCAGCGTGCAGGCGGAAGATCTGGCGGTGTATTATTGCCATCAGTATCTG AGCAGCTGGACCTTTGGCGGCGGCACCAAACTGGAAATTAAACCGAAAAGCTGCGATA AAACCCATACCTGCCCGCCGTGCCCG GCGCCGGAATTTCAGGGCGGCCCGAGCGTGTTTCTGTTTCCGCCGAAACCGAAAGATA CCCTGTATATTACCCGCGAACCGGAAGTGACCTGCGTGGTGGTGGATGTGAGCCATGA AGATCCGGAAGTGAAATTTAACTGGTATGTGGATGGCGTGGAAGTGCATAACGCGAAA ACCAAACCGCGCGAAGAACAGTATAACAGCACCTATCGCGTGGTGAGCGTGCTGACCG TGCTGCATCAGGATTGGCTGAACGGCAAAGAATATAAATGCCAGGTGAGCAACAAAGC GCTGCCGGCGCCGATTGAAAAAACCATTAGCAAAGCGAAAGGCCAGCCGCGCGAACC GCAGGTGTATGTGTATCCGCCGAGCCGCGATGAACTGACCAAAAACCAGGTGAGCCTG ACCTGCCTGGTGAAAGGCTTTTATCCGAGCGATATTGCGGTGGAATGGGAAAGCAACG GCCAGCCGGAAAACAACTATAAAACCACCCCGCCGGTGCTGGATAGCGATGGCAGCTT TGCGCTGGTGAGCAAACTGACCGTGGATAAAAGCCGCTGGCAGCAGGGCAACGTGTTT AGCTGCAGCGTGATGCATGAAGCGCTGCATAACCATTATACCCAGAAAAGCCTGAGCC TGAGCCCGGGC (SEQ ID NO: 38). In some embodiments, the nucleic acid sequence encoding the anti-CD45 portion (scFv-Fc) of an anti-CD45×SLAMF6 bispecific antibody comprises SEQ ID NO: 3 immediately followed by SEQ ID NO: 38. In some embodiments, the signal peptide is cleaved during post-translational modifications that occur in vitro or in vivo.

In some embodiments, the nucleic acid sequence of the anti-CD45 portion (scFv-Fc) of the anti-CD45×SLAMF6 bispecific antibody comprises a nucleic acid sequence encoding a signal peptide comprising SEQ ID NO: 3. In some embodiments, the nucleic acid sequence encoding the anti-CD45 portion (scFv-Fc) of an anti-CD45×SLAMF6 bispecific antibody (without the signal peptide) comprises SEQ ID NO: 39. GAAGTGCAGCTGCAGCAGAGCGGCGCGGAACTGGTGAAACCGGGCGCGAGCGTGAAA CTGAGCTGCACCGCGAGCGGCTTTAACATTAAAGATACCTTTATGCATTGGGTGAAACT GCGCCCGGAACAGGGCCTGGAATGGATTGGCCGCATTGATCCGGCGAACGGCTATACC AAATATGATCCGCGCTTTCAGGGCAAAGCGACCATTATTGCGGATACCAGCAGCAACA CCGCGTATCTGCAGCTGAGCAGCCTGACCAGCGAAGATACCGCGGTGTATTATTGCGC GAGCGGCGAATATTATGCGCTGGATTATTGGGGCCAGGGCACCAGCGTGACCGTGAGC AGCGGCGGCGGCGGCAGCGGCGGCGGCGGCAGCGGCGGCGGCGGCAGCGATATTGTG CTGACCCAGAGCCCGGCGAGCCTGACCGTGAGCCTGGGCCAGCGCGCGACCATTAGCT GCCGCGCGAGCAAAAGCGTGAGCACCAGCGGCTATAGCTATATGCATTGGTATCAGCA GAAACCGGGCCAGCCGCCGAAACTGCTGATTTATCTGGCGAGCAACCTGGAAAGCGGC GTGCCGGCGCGCTTTAGCGGCAGCGGCAGCGGCACCGATTTTACCCTGAACATTCATCC GGTGGAAGAAGAAGATGCGGCGACCTATTATTGCCAGCATAACCGCGAACTGCCGTAT ACCTTTGGCGGCGGCACCAAACTGGAAATTAAACCGAAAAGCTGCGATAAAACCCATA CCTGCCCGCCGTGCCCGGCGCCGGAATTTCAGGGCGGCCCGAGCGTGTTTCTGTTTCCG CCGAAACCGAAAGATACCCTGTATATTACCCGCGAACCGGAAGTGACCTGCGTGGTGG TGGATGTGAGCCATGAAGATCCGGAAGTGAAATTTAACTGGTATGTGGATGGCGTGGA AGTGCATAACGCGAAAACCAAACCGCGCGAAGAACAGTATAACAGCACCTATCGCGT GGTGAGCGTGCTGACCGTGCTGCATCAGGATTGGCTGAACGGCAAAGAATATAAATGC CAGGTGAGCAACAAAGCGCTGCCGGCGCCGATTGAAAAAACCATTAGCAAAGCGAAA GGCCAGCCGCGCGAACCGCAGGTGTATGTGTATCCGCCGAGCCGCGATGAACTGACCA AAAACCAGGTGAGCCTGACCTGCCTGGTGAAAGGCTTTTATCCGAGCGATATTGCGGT GGAATGGGAAAGCAACGGCCAGCCGGAAAACAACTATAAAACCACCCCGCCGGTGCT GGATAGCGATGGCAGCTTTGCGCTGGTGAGCAAACTGACCGTGGATAAAAGCCGCTGG CAGCAGGGCAACGTGTTTAGCTGCAGCGTGATGCATGAAGCGCTGCATAACCATTATA CCCAGAAAAGCCTGAGCCTGAGCCCGGGC (SEQ ID NO: 39). In some embodiments, the nucleic acid sequence encoding the anti-CD45 portion (scFv-Fc) of an anti-CD45×SLAMF6 bispecific antibody comprises SEQ ID NO: 3 immediately followed by SEQ ID NO: 39. In some embodiments, the signal peptide is cleaved during post-translational modifications that occur in vitro or in vivo.

In some embodiments, the nucleic acid sequence encoding a signal peptide of the anti-CD45 portion (scFv-Fc) of the anti-CD45×SLAMF6 bispecific antibody comprise a nucleic acid sequence 80, 81, 82, 83, 84, 85, 86, 87, 88, 89, 90, 91, 92, 93, 94, 95, 96, 97, 98 or 99% identical to SEQ ID NO: 3. In some embodiments, the nucleic acid sequence encoding a anti-CD45 portion (scFv-Fc) of the anti-CD45×SLAMF6 bispecific antibody (without the signal peptide) comprise a nucleic acid sequence 80, 81, 82, 83, 84, 85, 86, 87, 88, 89, 90, 91, 92, 93, 94, 95, 96, 97, 98 or 99% identical to SEQ ID NO: 6. In some embodiments, the nucleic acid sequence encoding any of the anti-CD45 portions (scFv-Fc) of the anti-CD45×SLAMF6 bispecific antibody (without the signal peptide) comprise a nucleic acid sequence 80, 81, 82, 83, 84, 85, 86, 87, 88, 89, 90, 91, 92, 93, 94, 95, 96, 97, 98 or 99% identical to SEQ ID NOs: 34-39. In some embodiments, the nucleic acid sequence encodes CDRs of the variable heavy chain domain and the CDRs of the variable light chain domain of the anti-CD45 portion (scFv-Fc) of the anti-CD45×SLAMF6 bispecific antibody wherein the CDR sequences are indicated above (in bold and underline). In some embodiments, the nucleic acid sequence encoding framework regions (FRs) of the variable heavy chain domain and the FRs of the variable light chain domain of the anti-CD45 portion of the anti-CD45×SLAMF6 bispecific antibody comprise a nucleic acid sequence 80, 81, 82, 83, 84, 85, 86, 87, 88, 89, 90, 91, 92, 93, 94, 95, 96, 97, 98 or 99% identical to the FRs of SEQ ID NO: 6. In some embodiments, the nucleic acid sequence encoding framework regions (FRs) of the variable heavy chain domain and the FRs of the variable light chain domain of the anti-CD45 portion of the anti-CD45×SLAMF6 bispecific antibody comprise a nucleic acid sequence 80, 81, 82, 83, 84, 85, 86, 87, 88, 89, 90, 91, 92, 93, 94, 95, 96, 97, 98 or 99% identical to the FRs of SEQ ID NOs: 34-39.

Although the sequence above is depicted with a nucleic acid sequence encoding a linker “GGAGGCGGAGGTTCTGGCGGCGGAGGAAGTGGTGGCGGAGGCTCT” (SEQ ID NO: 15) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker comprises a nucleic acid sequence encoding a linker that is between about 2 and 25 amino acids in length. In some embodiments, the nucleic acid sequence encodes a glycine-serine linker that is about 15 amino acids in length.

### Amino Acid Sequences of Anti-CD43 Portion of Anti-CD43×SLAMF6 Bispecific Antibodies

In some embodiments, the anti-CD43 portion of the anti-CD43×SLAMF6 bispecific antibody comprises a single-chain variable fragment (scFv) with a fragment crystallizable (Fc) region (i.e., scFv-Fc antibody) comprising a signal peptide, a variable heavy chain domain, a linker, a variable light chain domain, and an Fc portion. Underlined and bolded amino acids represent the respective complementarity determining regions (CDRs). Double underlined amino acids represent a linker sequence between the variable heavy chain and the variable light chain portions. Italicized amino acids represent the Fc portion. Underlined amino acids represent the fragment, crystallizable (Fc) region. Bolded, underlined, and italicized amino acids represent the “hole” mutations for the production of knob-in-hole antibodies. In some embodiments, instead of the “hole” mutations, the amino acid sequence comprising the anti-CD43 portion of an anti-CD43×SLAMF6 bispecific antibody can comprise the “knob” mutations, while the “hole” mutations are present on an anti-SLAMF6 portion of an anti-CD43×SLAMF6 bispecific antibody.

In some embodiments, the amino acid sequence of the anti-CD43 portion (scFv-Fc) of the anti-CD43×SLAMF6 bispecific antibody comprises a signal peptide comprising SEQ ID NO: 17. MGWSCIILFLVATATGVHS (SEQ ID NO: 1). In some embodiments, the amino acid sequence of the anti-CD43 portion (scFv-Fc) of an anti- anti-CD43×SLAMF6 bispecific antibody (without the signal peptide) comprises SEQ ID NO: 7.

(SEQ ID NO: 7)
EVQLVESGGGLVQPGGSLRLSCVASGFTFSSFGMHWVRQAPGKGLEWVA
YISSGSGNFYYVDTVKGRFTISRDNAKNSLYLQMNSLRAEDTAVYYCAR
STYYHGSRGAMDYWGQGTLVTVSSGGGGSGGGGSGGGGSDVVMTQSPAF
LSVTPGEKVTITCSASSSVSSMYWYQQKPDQAPKLLIYDTSKMASGVPS
RFSGSGSGTDFTFTISSLEAEDAATYYCQQWSSYPPITFGGGTKLEIKR
LEPKSCDKTHTCPPCPAPEFQGGPSVFLFPPKPKDTLYITREPEVTCVV
VDVSHEDPEVKFNWYVDGVEVHNAKTKPREEQYNSTYRVVSVLTVLHQD
WLNGKEYKCQVSNKALPAPIEKTISKAKGQPREPQVYVYPPSRDELTKN
QVSLTCLVKGFYPSDIAVEWESNGQPENNYKTTPPVLDSDGSFALVSKL
TVDKSRWQQGNVFSCSVMHEALHNHYTQKSLSLSPG.

In some embodiments, the amino acid sequence of the anti-CD43 portion (scFv-Fc) of an anti-CD43×SLAMF6 bispecific antibody comprises SEQ ID NO: 1 immediately followed by SEQ ID NO: 7. In some embodiments, the signal peptide is cleaved during post-translational modifications that occur in vitro or in vivo.

In some embodiments, the signal peptide of the anti-CD43 portion (scFv-Fc) of the anti-CD43×SLAMF6 bispecific antibody comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-CD43 portion (scFv-Fc) of the anti-CD43×SLAMF6 bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 7. In some embodiments, the anti-CD43 portion of an anti-CD43×SLAMF6 bispecific antibody comprises an amino acid sequence comprising the CDRs of the variable heavy chain domain and the CDRs of the variable light chain domain of the anti-CD43 portion (scFv-Fc) of the anti-CD43×SLAMF6 bispecific antibody wherein the CDR sequences are indicated above (in bold and underline). In some embodiments, the framework regions (FRs) of the variable heavy chain domain and the FRs of the variable light chain domain of the anti-CD43 portion of the anti-CD43×SLAMF6 bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 7.

Although the sequence above is depicted with a linker “GGGGSGGGGSGGGGS” (SEQ ID NO: 14) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker is between about 2 and 25 amino acids in length. In some embodiments, the glycine-serine linker is about 15 amino acids in length.

### Nucleic Acid Sequences of Anti-CD43 Portion of Anti-CD43×SLAMF6 Bispecific Antibodies

The amino acid sequences of the respective CDRs, linker portions, VH portion, VL portion, Fc portion, and knob/hole mutations are provided herein. Nucleic acid sequences encoding the respective CDRs, linker portions, VH portion, VL portion, Fc portion and knob/hole mutations can be determined from the amino acid sequence by a person of skill in the art. Nucleic acid sequences encoding the respective CDRs, linker portions, VH portion, VL portion, Fc portion and knob/hole mutations in SEQ ID NO:8 can be determined from the amino acid sequence of SEQ ID NO: 7 by a person of skill in the art. In some embodiments, instead of the “hole” mutations, the nucleic acid sequence comprising the anti-CD43 portion of an anti-CD43×SLAMF6 bispecific antibody can comprise a nucleic acid sequence encoding the “knob” mutations, while the “hole” mutations are present on an anti-SLAMF6 portion of an anti-CD43×SLAMF6 bispecific antibody. In some embodiments, the nucleic acid sequences may be codon optimized.

In some embodiments, the nucleic acid sequence of the anti-CD43 portion (scFv-Fc) of the anti-CD43×SLAMF6 bispecific antibody comprises a signal peptide comprising SEQ ID NO: 3. ATGGGCTGGAGCTGCATTATCCTGTTCCTGGTGGCCACAGCCACCGGCGTGCACAGC (SEQ ID NO: 3). In some embodiments, the nucleic acid sequence of the anti-CD43 portion (scFv-Fc) of an anti-CD43×SLAMF6 bispecific antibody (without the signal peptide) comprises SEQ ID NO: 8. AGGTGCAGCTGGTTGAATCTGGCGGAGGACTGGTTCAGCCTGGCGGATCTCTGAGACT GAGCTGTGTGGCCAGCGGCTTCACCTTCAGCAGCTTTGGCATGCACTGGGTCCGACAG GCCCCTGGCAAAGGACTTGAGTGGGTCGCCTACATCAGCAGCGGCAGCGGCAACTTCT ACTACGTGGACACCGTGAAGGGCAGATTCACCATCTCCAGAGACAACGCCAAGAACAG CCTGTACCTGCAGATGAACTCCCTGAGAGCCGAGGACACCGCCGTGTACTACTGTGCC AGAAGCACCTACTACCACGGCAGCAGAGGCGCCATGGATTATTGGGGACAGGGCACCC TGGTCACCGTTTCTAGCGGAGGCGGAGGTTCTGGCGGCGGAGGAAGTGGTGGCGGAGG CTCTGATGTGGTCATGACTCAGAGCCCTGCCTTCCTGTCTGTGACCCCTGGCGAGAAAG TGACCATCACCTGTAGCGCCAGCAGCAGCGTGTCCAGCATGTACTGGTATCAGCAGAA GCCCGATCAGGCTCCCAAACTGCTGATCTACGACACCAGCAAGATGGCCTCTGGCGTG CCCAGCAGATTTTCTGGCTCTGGCAGCGGAACCGACTTCACCTTTACCATCAGCTCCCT GGAAGCCGAAGATGCCGCCACCTACTATTGCCAGCAGTGGTCTAGCTACCCTCCTATCA CCTTTGGAGGCGGCACCAAGCTGGAAATCAAGCGGCTCGAGCCCAAGAGCTGCGACAA GACCCACACCTGTCCTCCATGTCCTGCTCCAGAGTTTCAAGGCGGCCCTTCCGTGTTCC TGTTTCCTCCAAAGCCTAAGGACACCCTGTACATCACCCGCGAGCCTGAAGTGACCTGT GTGGTGGTGGATGTGTCCCACGAGGACCCCGAAGTGAAGTTCAATTGGTACGTGGACG GCGTGGAAGTGCACAACGCCAAGACCAAGCCTAGAGAGGAACAGTACAACAGCACCT ACAGAGTGGTGTCCGTGCTGACCGTGCTGCACCAGGATTGGCTGAACGGCAAAGAGTA CAAGTGCCAGGTGTCCAACAAGGCCCTGCCTGCTCCTATCGAGAAAACCATCAGCAAG GCCAAGGGCCAGCCTAGGGAACCTCAAGTGTACGTGTACCCTCCTAGCCGGGACGAGC TGACCAAGAATCAGGTGTCCCTGACCTGCCTCGTGAAGGGCTTCTACCCTTCCGACATC GCCGTGGAATGGGAGAGCAATGGCCAGCCTGAGAACAACTACAAGACAACCCCTCCTG TGCTGGACAGCGACGGCTCTTTTGCCCTGGTGTCCAAGCTGACAGTGGACAAGTCCAG ATGGCAGCAGGGCAACGTGTTCAGCTGCAGCGTGATGCACGAGGCCCTGCACAACCAC TACACCCAGAAGTCCCTGAGCCTGTCTCCTGGATGA (SEQ ID NO: 8). In some embodiments, the nucleic acid sequence encoding the anti-CD43 portion (scFv-Fc) of an anti-CD43×SLAMF6 bispecific antibody comprises SEQ ID NO: 3 immediately followed by SEQ ID NO: 8. In some embodiments, the signal peptide is cleaved during post-translational modifications that occur in vitro or in vivo.

In some embodiments, the nucleic acid sequence encoding a signal peptide of the anti-CD43 portion (scFv-Fc) of the anti-CD43×SLAMF6 bispecific antibody comprises a nucleic acid sequence 80, 81, 82, 83, 84, 85, 86, 87, 88, 89, 90, 91, 92, 93, 94, 95, 96, 97, 98 or 99% identical to SEQ ID NO: 3. In some embodiments, the nucleic acid sequence encoding an anti-CD43 portion (scFv-Fc) of the anti-CD43×SLAMF6 bispecific antibody (without the signal peptide) comprises a nucleic acid sequence 80, 81, 82, 83, 84, 85, 86, 87, 88, 89, 90, 91, 92, 93, 94, 95, 96, 97, 98 or 99% identical to SEQ ID NO: 8. In some embodiments, the nucleic acid sequence encodes CDRs of the variable heavy chain domain and the CDRs of the variable light chain domain of the anti-CD43 portion (scFv-Fc) of the anti-CD43×SLAMF6 bispecific antibody wherein the CDR sequences are indicated above (in bold and underline). In some embodiments, the nucleic acid sequence encoding framework regions (FRs) of the variable heavy chain domain and the FRs of the variable light chain domain of the anti-CD43 portion of the anti-CD43×SLAMF6 bispecific antibody comprise a nucleic acid sequence 80, 81, 82, 83, 84, 85, 86, 87, 88, 89, 90, 91, 92, 93, 94, 95, 96, 97, 98 or 99% identical to the FRs of SEQ ID NO: 8.

Although the sequence above is depicted with a nucleic acid sequence encoding a linker “GGAGGCGGAGGTTCTGGCGGCGGAGGAAGTGGTGGCGGAGGCTCT” (SEQ ID NO: 15) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker comprises a nucleic acid sequence encoding a linker that is between about 2 and 25 amino acids in length. In some embodiments, the nucleic acid sequence encodes a glycine-serine linker that is about 15 amino acids in length.

### Amino Acid Sequences of Anti-SLAMF6 Portions of Anti-CD3×SLAMF6, Anti-CD45×SLAMF6, or Anti-CD43×SLAMF6 Bispecific Antibodies

In some embodiments, the anti-SLAMF6 portion of the anti-CD3×SLAMF6, anti-CD45×SLAMF6, or anti-CD43×SLAMF6 bispecific antibody comprises a single-chain variable fragment (scFv) with a fragment crystallizable (Fc) region (i.e., scFv-Fc antibody) comprising a signal peptide, a variable heavy chain domain, a linker, a variable light chain domain, and an Fc portion. Underlined and bolded amino acids represent the respective complementarity determining regions (CDRs). Double underlined amino acids represent a linker sequence between the variable heavy chain and the variable light chain portions. Italicized amino acids represent the Fc portion. Underlined amino acids represent the fragment, crystallizable (Fc) region. Bolded, underlined, and italicized amino acids represent the “knob” mutations for the production of knob-in-hole antibodies. In some embodiments, instead of the “knob” mutations, the amino acid sequence comprising the anti-SLAMF6 portion of an anti-CD45×SLAMF6 bispecific antibody can comprise the “hole” mutations, while the “knob” mutations are present on an anti-CD3 portion of an anti-CD3×SLAMF6 bispecific antibody, an anti-CD45 portion of an anti-CD45×SLAMF6 bispecific antibody, or an anti-CD43 portion of an anti-CD43×SLAMF6 bispecific antibody.

In some embodiments, the amino acid sequence of the anti-SLAMF6 portion (scFv-Fc) of an antiCD3×SLAMF6, an anti-CD45×SLAMF6, or an anti-CD43×SLAMF6 bispecific antibody comprises a signal peptide comprising SEQ ID NO: 1. MGWSCIILFLVATATGVHS (SEQ ID NO: 1). In some embodiments, the amino acid sequence of the anti-SLAMF6 portion (scFv-Fc) of an anti-CD3×SLAMF6, an anti-CD45×SLAMF6, or anti-CD43×SLAMF6 bispecific antibody (without the signal peptide) comprises SEQ ID NO: 9.

(SEQ ID NO: 9)
QVQLVQSGSELKKPGASVKVSCKASGYTFTNFGMNWVRQAPGQGLEWMG
WINTYSGEATYADDFKGRFVFSLDTSVSTAYLQISSLKAEDTAVYYCAR
RGGTAEFDYWGQGTLVTVSSGGGGSGGGGSGGGGSEIVLTQSPDFQSVT
PKEKVTITCSASSSISSNFLHWYQQKPDQSPKLLIYRTSKLASGVPSRF
SGSGSGTDFTLTINSLEAEDAATYYCQQGIYMPLTFGGGTKLEIKRLEP
KSCDKTHTCPPCPAPEFQGGPSVFLFPPKPKDTLYITREPEVTCVVVDV
SHEDPEVKFNWYVDGVEVHNAKTKPREEQYNSTYRVVSVLTVLHQDWLN
GKEYKCQVSNKALPAPIEKTISKAKGQPREPQVYVLPPSRDELTKNQVS
LLCLVKGFYPSDIAVEWESNGQPENNYLTWPPVLDSDGSFFLYSKLTVD
KSRWQQGNVFSCSVMHEALHNHYTQKSLSLSPG.

In some embodiments, the amino acid sequence of the anti-SLAMF6 portion (scFv-Fc) of an anti-CD3×SLAMF6, an anti-CD45×SLAMF6, or anti-CD43×SLAMF6 bispecific antibody comprises SEQ ID NO: 1 immediately followed by SEQ ID NO: 9. In some embodiments, the signal peptide is cleaved during post-translational modifications that occur in vitro or in vivo.

In some embodiments, the signal peptide of the anti-SLAMF6 portion (scFv-Fc) of the antiCD3×SLAMF6, the anti-CD45×SLAMF6, or the anti-CD43×SLAMF6 bispecific antibody comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-SLAMF6 portion (scFv-Fc) of the antiCD3×SLAMF6, the anti-CD45×SLAMF6, or the anti-CD43×SLAMF6 bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 9. In some embodiments, the anti-SLAMF6 portion of an anti-CD3×SLAMF6, an anti-CD45×SLAMF6, or an anti-CD43×SLAMF6 bispecific antibody comprises an amino acid sequence comprising the CDRs of the variable heavy chain domain and the CDRs of the variable light chain domain of the anti-SLAMF6 portion (scFv-Fc) of the antiCD3×SLAMF6, the anti-CD45×SLAMF6, or the anti-CD43×SLAMF6 bispecific antibody wherein the CDR sequences are indicated above (in bold and underline). In some embodiments, the framework regions (FRs) of the variable heavy chain domain and the FRs of the variable light chain domain of the anti-SLAMF6 portion of the antiCD3×SLAMF6, the anti-CD45×SLAMF6, or anti-CD43×SLAMF6 bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 9.

Although the sequence above is depicted with a linker “GGGGSGGGGSGGGGS” (SEQ ID NO: 14) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker is between about 2 and 25 amino acids in length. In some embodiments, the glycine-serine linker is about 15 amino acids in length.

### Nucleic Acid Sequences of Anti-SLAMF6 Portions of Anti-CD3×SLAMF6, Anti-CD45×SLAMF6, or Anti-CD43×SLAMF6 Bispecific Antibodies

The amino acid sequences of the respective CDRs, linker portions, VH portion, VL portion, Fc portion, and knob/hole mutations are provided herein. Nucleic acid sequences encoding the respective CDRs, linker portions, VH portion, VL portion, Fc portion and knob/hole mutations can be determined from the amino acid sequence by a person of skill in the art. Nucleic acid sequences encoding the respective CDRs, linker portions, VH portion, VL portion, Fc portion and knob/hole mutations in SEQ ID NO:10 can be determined from the amino acid sequence of SEQ ID NO: 9 by a person of skill in the art. In some embodiments, instead of the “knob” mutations, the nucleic acid sequence comprising the anti-SLAMF6 portion of an antiCD3×SLAMF6, an anti-CD45×SLAMF6, or an antiCD3×SLAMF6 bispecific antibody can comprise the “hole” mutations, while the “knob” mutations are present on an anti-CD3 portion of an anti-CD3×SLAMF6 bispecific antibody, an anti-CD45 portion of an anti-CD45×SLAMF6 bispecific antibody, or an anti-CD43 portion of an anti-CD43×SLAMF6 bispecific antibody.

In some embodiments, the nucleic acid sequence encoding the anti-SLAMF6 portion (scFv-Fc) of the antiCD3×SLAMF6, the anti-CD45×SLAMF6, or the anti-CD43×SLAMF6 bispecific antibody comprises a signal peptide comprising SEQ ID NO: 3. ATGGGCTGGAGCTGCATTATCCTGTTCCTGGTGGCCACAGCCACCGGCGTGCACAGC (SEQ ID NO: 3). In some embodiments, the nucleic acid sequence encoding the anti-SLAMF6 portion (scFv-Fc) of an anti-CD3×SLAMF6, an anti-CD45×SLAMF6, or an anti-CD43×SLAMF6 bispecific antibody (without the signal peptide) comprises SEQ ID NO: 10. CCAGGTGCAGCTGGTGCAGAGCGGAGCAGAGGTGAAGAAGCCAGGAGCCTCTGTGAA GGTGAGCTGCAAGGCCTCCGGCTACACCTTCACACGGTATACCATGCACTGGGTGAGA CAGGCACCTGGACAGGGCCTGGAGTGGATGGGCTACATCAACCCAAGCCGGGGCTACA CAAACTATAATCAGAAGTTTAAGGACAGAGTGACCATCACAGCCGATAAGAGCACCTC CACAGCCTATATGGAGCTGAGCTCCCTGAGGTCCGAGGACACCGCCGTGTACTATTGC GCCCGCTACTATGACGATCACTACTGTCTGGATTATTGGGGCCAGGGCACCACAGTGA CAGTGTCTAGCGGCGGAGGAGGCTCTGGAGGAGGAGGCAGCGGCGGCGGCGGCTCTG GCAGCGAGATCGTGCTGACCCAGTCCCCAGCCACACTGTCCCTGTCTCCAGGAGAGAG GGCCACCCTGAGCTGCTCCGCCTCCTCTAGCGTGTCTTACATGAATTGGTATCAGCAGA AGCCCGGCAAGGCCCCTAAGAGGCTGATCTACGACACCTCTAAGCTGGCAAGCGGAGT GCCCTCCCGCTTCTCTGGCAGCGGCTCCGGCACCGACTTTACCCTGACAATCAACTCCC TGGAGGCCGAGGATGCCGCCACATACTATTGTCAGCAGTGGTCCTCTAATCCTTTCACC TTTGGCCAGGGCACAAAGGTGGAGATCAAGCGGCTCGAGCCCAAGAGCTGCGACAAG ACCCACACCTGTCCTCCATGTCCTGCTCCAGAGTTTCAAGGCGGCCCTTCCGTGTTCCT GTTTCCTCCAAAGCCTAAGGACACCCTGTACATCACCCGCGAGCCTGAAGTGACCTGTG TGGTGGTGGATGTGTCCCACGAGGACCCCGAAGTGAAGTTCAATTGGTACGTGGACGG CGTGGAAGTGCACAACGCCAAGACCAAGCCTAGAGAGGAACAGTACAACAGCACCTA CAGAGTGGTGTCCGTGCTGACCGTGCTGCACCAGGATTGGCTGAACGGCAAAGAGTAC AAGTGCCAGGTGTCCAACAAGGCCCTGCCTGCTCCTATCGAGAAAACCATCAGCAAGG CCAAGGGCCAGCCTAGGGAACCTCAAGTGTACGTGTACCCTCCTAGCCGGGACGAGCT GACCAAGAATCAGGTGTCCCTGACCTGCCTCGTGAAGGGCTTCTACCCTTCCGACATCG CCGTGGAATGGGAGAGCAATGGCCAGCCTGAGAACAACTACAAGACAACCCCTCCTGT GCTGGACAGCGACGGCTCTTTTGCCCTGGTGTCCAAGCTGACAGTGGACAAGTCCAGA TGGCAGCAGGGCAACGTGTTCAGCTGCAGCGTGATGCACGAGGCCCTGCACAACCACT ACACCCAGAAGTCCCTGAGCCTGTCTCCTGGATGA (SEQ ID NO: 10). In some embodiments, the nucleic acid sequence encoding the anti-SLAMF6 portion (scFv-Fc) of an anti-CD3×SLAMF6, an anti-CD45×SLAMF6, or anti-CD43×SLAMF6 bispecific antibody comprises SEQ ID NO: 3 immediately followed by SEQ ID NO: 10. In some embodiments, the signal peptide is cleaved during post-translational modifications that occur in vitro or in vivo.

In some embodiments, the nucleic acid sequence encoding a signal peptide of the anti-SLAMF6 portion (scFv-Fc) of the anti-CD3×SLAMF6, the anti-CD45×SLAMF6, or anti-CD43×SLAMF6 bispecific antibody comprises an nucleic acid sequence 80, 81, 82, 83, 84, 85, 86, 87, 88, 89, 90, 91, 92, 93, 94, 95, 96, 97, 98 or 99% identical to SEQ ID NO: 3. In some embodiments, the nucleic acid sequence encoding an anti-SLAMF6 portion (scFv-Fc) of the anti-CD3×SLAMF6, the anti-CD45×SLAMF6, or the anti-CD43×SLAMF6 bispecific antibody (without the signal peptide) comprises an nucleic acid sequence 80, 81, 82, 83, 84, 85, 86, 87, 88, 89, 90, 91, 92, 93, 94, 95, 96, 97, 98 or 99% identical to SEQ ID NO: 10. In some embodiments, the nucleic acid sequence encodes CDRs of the variable heavy chain domain and the CDRs of the variable light chain domain of the anti-SLAMF6 portion (scFv-Fc) of the anti-CD3×SLAMF6, the anti-CD45×SLAMF6, or the anti-CD43×SLAMF6 bispecific antibody wherein the CDR sequences are indicated above (in bold and underline). In some embodiments, the nucleic acid sequence encoding framework regions (FRs) of the variable heavy chain domain and the FRs of the variable light chain domain of the anti-SLAMF6 portion of the anti-CD3×SLAMF6, the anti-CD45×SLAMF6, or the anti-CD43×SLAMF6 bispecific antibody comprise an nucleic acid sequence 80, 81, 82, 83, 84, 85, 86, 87, 88, 89, 90, 91, 92, 93, 94, 95, 96, 97, 98 or 99% identical to the FRs of SEQ ID NO: 10.

Although the sequence above is depicted with a nucleic acid sequence encoding a linker “GGAGGCGGAGGTTCTGGCGGCGGAGGAAGTGGTGGCGGAGGCTCT” (SEQ ID NO: 15) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker comprises a nucleic acid sequence encoding a linker that is between about 2 and 25 amino acids in length. In some embodiments, the nucleic acid sequence encodes a glycine-serine linker that is about 15 amino acids in length.

### Additional Amino Acid and Nucleic Acid Sequences of An Anti-SLAMF6 Portion of Anti-CD3×SLAMF6, Anti-CD45×SLAMF6, or Anti-CD43×SLAMF6 Bispecific Antibodies

In some embodiments, the anti-SLAMF6 portion of the anti-CD3×SLAMF6, the anti-CD45×SLAMF6, or the anti-CD43×SLAMF6 bispecific antibody comprises a single-chain variable fragment (scFv) with a fragment crystallizable (Fc) region (i.e., scFv-Fc antibody). In some embodiments, the anti-SLAMF6 portion of the anti-CD3×SLAMF6, the anti-CD45×SLAMF6, or the anti-CD43×SLAMF6 bispecific antibody comprises a signal peptide, a variable heavy chain domain, a linker, a variable light chain domain, and an Fc portion. In some embodiments, the amino acid sequence of the anti-SLAMF6 portion of the anti-CD3×SLAMF6, the anti-CD45×SLAMF6, or the anti-CD43×SLAMF6 bispecific antibody comprises at least a portion of the amino acid sequence of any anti-SLAMF6 antibody known in the art. In some embodiments, the amino acid sequence of the anti-SLAMF6 portion of the anti-CD3×SLAMF6, the anti-CD45×SLAMF6, or the anti-CD43×SLAMF6 bispecific antibody comprises at least a variable heavy and a variable light chain portion of the amino acid sequence of any anti-SLAMF6 antibody known in the art. In some embodiments, the amino acid sequence of the anti-SLAMF6 portion of the anti-CD3×SLAMF6, the anti-CD45×SLAMF6, or the anti-CD43×SLAMF6 bispecific antibody comprises at least the CDRs of the variable heavy chain and the CDRs of the variable light chain portions of the amino acid sequence of any anti-SLAMF6 antibody known in the art In some embodiments, the nucleic acid sequence the anti-SLAMF6 portion of the anti-CD3×SLAMF6, the anti-CD45×SLAMF6, or the anti-CD43×SLAMF6 bispecific antibody codes for an amino acid sequence that comprises at least a portion of the amino acid sequence of any anti-SLAMF6 antibody known in the art. In some embodiments, the nucleic acid sequence of the anti-SLAMF6 portion of the anti-CD3×SLAMF6, the anti-CD45×SLAMF6, or the anti-CD43×SLAMF6 bispecific antibody codes for an amino acid sequence that comprises at least a variable heavy and a variable light chain portion of the amino acid sequence of any anti-SLAMF6 antibody known in the art. In some embodiments, the nucleic acid sequence encoding the anti-SLAMF6 portion of the anti-CD3×SLAMF6, the anti-CD45×SLAMF6, or the anti-CD43×SLAMF6 bispecific antibody codes for an amino acid sequence that comprises at least the CDRs of the variable heavy chain and the CDRs of the variable light chain portions of the amino acid sequence of any anti-SLAMF6 antibody known in the art.

### Amino Acid Sequences of Anti-CD3 Portions of Anti-CD3×SLAMF6 Tetravalent Bispecific Antibodies

In some embodiments, the anti-CD3 portion of the anti-CD3×SLAMF6 tetravalent bispecific antibody is a first single-chain variable fragment (scFv) with a fragment crystallizable (Fc) region and a second single-chain variable fragment (scFv) (i.e., scFv-Fc-scFv antibody) comprising a signal peptide, a first variable heavy chain (VH) domain, a first linker, a first variable light chain (VL) domain, an Fc portion, a second variable) domain. Underlined and bolded amino acids represent the respective complementarity determining regions (CDRs). Double underlined amino acids represent a linker sequence between the variable heavy chain and the variable light chain portions. Underlined amino acids represent the fragment, crystallizable (Fc) region. Bolded, underlined, and italicized amino acids represent the “hole” mutations for the production of knob-in-hole antibodies. In some embodiments, instead of the “hole” mutations, the amino acid sequence comprising the anti-CD3 portion of an anti-CD3×SLAMF6 tetravalent bispecific antibody can comprise the “knob” mutations, while the “hole” mutations are present on an anti-SLAMF6 portion of an anti-CD3×SLAMF6 tetravalent bispecific antibody.

In some embodiments, the amino acid sequence of the anti-CD3 portion (scFv-Fc-scFv) of the anti-CD3×SLAMF6 tetravalent bispecific antibody comprises a signal peptide comprising SEQ ID NO: 1. MGWSCIILFLVATATGVHS (SEQ ID NO: 1). In some embodiments, the amino acid sequence of the anti-CD3 portion (scFv-Fc-scFv) of an anti-CD3×SLAMF6 tetravalent bispecific antibody (without the signal peptide) comprises SEQ ID NO: 11.

(SEQ ID NO: 11)
QVQLVQSGAEVKKPGASVKVSCKASGYTFTRYTMHWVRQAPGQGLEWMGYINPSRGY
TNYNQKFKDRVTITADKSTSTAYMELSSLRSEDTAVYYCARYYDDHYCLDYWGQGTTV
TVSSGGGGSGGGGSGGGGSGSEIVLTQSPATLSLSPGERATLSCSASSSVSYMNWYQQKPG
KAPKRLIYDTSKLASGVPSRFSGSGSGTDFTLTINSLEAEDAATYYCQQWSSNPFTFGQGT
KVEIKRLEPKSCDKTHTCPPCPAPEFQGGPSVFLFPPKPKDTLYITREPEVTCVVVDVSHEDP
EVKFNWYVDGVEVHNAKTKPREEQYNSTYRVVSVLTVLHQDWLNGKEYKCQVSNKALP
APIEKTISKAKGQPREPQVYVYPPSRDELTKNQVSLTCLVKGFYPSDIAVEWESNGQPENNY
KTTPPVLDSDGSFALVSKLTVDKSRWQQGNVFSCSVMHEALHNHYTQKSLSLSPGQVQLV
QSGAEVKKPGASVKVSCKASGYTFTRYTMHWVRQAPGQGLEWMGYINPSRGYTNYNQ
KFKDRVTITADKSTSTAYMELSSLRSEDTAVYYCARYYDDHYCLDYWGQGTTVTVSSGG
GGSGGGGSGGGGSGSEIVLTQSPATLSLSPGERATLSCSASSSVSYMNWYQQKPGKAPKR
LIYDTSKLASGVPSRFSGSGSGTDFTLTINSLEAEDAATYYCQQWSSNPFTFGQGTKVEIKR
LE.

In some embodiments, the amino acid sequence of the anti-CD3 portion (scFv-Fc-scFv) of an anti-CD3×SLAMF6 tetravalent bispecific antibody comprises SEQ ID NO: 1 immediately followed by SEQ ID NO: 11. In some embodiments, the signal peptide is cleaved during post-translational modifications that occur in vitro or in vivo.

In some embodiments, the signal peptide of the anti-CD3 portion (scFv-Fc-scFv) of the anti-CD3×SLAMF6 tetravalent bispecific antibody comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-CD3 portion (scFv-Fc-scFv) of the anti-CD3×SLAMF6 tetravalent bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 11. In some embodiments, the anti-CD3 portion of an anti-CD3×SLAMF6 tetravalent bispecific antibody comprises an amino acid sequence comprising the CDRs of the variable heavy chain domains and the CDRs of the variable light chain domains of the anti-CD3 portion of the anti-CD3×SLAMF6 tetravalent bispecific antibody wherein the CDR sequences are indicated above (in bold and underline). In some embodiments, the framework regions (FRs) of the variable heavy chain domains and the FRs of the variable light chain domains of the anti-CD3 portion of the anti-CD3×SLAMF6 tetravalent bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 11.

Although the sequence above is depicted with the linkers “GGGGSGGGGSGGGGS” (SEQ ID NO: 14) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker is between about 2 and 25 amino acids in length. In some embodiments, the glycine-serine linker is about 15 amino acids in length.

### Nucleic Acid Sequences of Anti-CD3 Portions of Anti-CD3×SLAMF6 Tetravalent Bispecific Antibodies

The amino acid sequences of the respective CDRs, linker portions, VH portion, VL portion, Fc portion, and knob/hole mutations are provided herein. Nucleic acid sequences encoding the respective CDRs, linker portions, VH portions, VL portions, Fc portion and knob/hole mutations can be determined from the amino acid sequence by a person of skill in the art. Nucleic acid sequences encoding the respective CDRs, linker portions, VH portion, VL portion, Fc portion and knob/hole mutations can be determined from the amino acid sequence of SEQ ID NO: 11 by a person of skill in the art. In some embodiments, instead of the “hole” mutations, the nucleic acid sequence comprising the anti-CD3 portion of an anti-CD3×SLAMF6 bispecific antibody can comprise a nucleic acid sequence encoding the “knob” mutations, while the “hole” mutations are present on an anti-SLAMF6 portion of an anti-CD3×SLAMF6 bispecific antibody. In some embodiments, the nucleic acid sequences encoding the respective CDRs, linker portions, VH portions, VL portions, Fc portion and knob/hole mutations may be codon optimized.

### Amino Acid Sequences of Anti-CD45 Portions of Anti-CD45×SLAMF6 Tetravalent Bispecific Antibodies

In some embodiments, the anti-CD45 portion of the anti-CD45×SLAMF6 tetravalent bispecific antibody is a first single-chain variable fragment (scFv) with a fragment crystallizable (Fc) region and a second single-chain variable fragment (scFv) (i.e., scFv-Fc-scFv antibody) comprising a signal peptide, a first variable heavy chain (VH) domain, a first linker, a first variable light chain (VL) domain, an Fc portion, a second variable heavy chain (VH) domain, a second linker, and a second variable light chain (VL) domain. Underlined and bolded amino acids represent the respective complementarity determining regions (CDRs). Double underlined amino acids represent a linker sequence between the variable heavy chain and the variable light chain portions. Underlined amino acids represent the fragment, crystallizable (Fc) region. Bolded, underlined, and italicized amino acids represent the “hole” mutations for the production of knob-in-hole antibodies. In some embodiments, instead of the “hole” mutations, the amino acid sequence comprising the anti-CD45 portion of an anti-CD45×SLAMF6 tetravalent bispecific antibody can comprise the “knob” mutations, while the “hole” mutations are present on an anti-SLAMF6 portion of an anti-CD45×SLAMF6 tetravalent bispecific antibody.

In some embodiments, the amino acid sequence of the anti-CD45 portion (scFv-Fc-scFv) of the anti-CD45×SLAMF6 tetravalent bispecific antibody comprises a signal peptide comprising SEQ ID NO: 1. MGWSCIILFLVATATGVHS (SEQ ID NO: 1). In some embodiments, the amino acid sequence of the anti-CD45 portion (scFv-Fc-scFv) of an anti-CD3×SLAMF45 tetravalent bispecific antibody (without the signal peptide) comprises SEQ ID NO: 12.

(SEQ ID NO: 12)
EVQLVESGGGLVQPGGSLRLSCAASGFTFNNYWMTWVRQAPGKGLEWVASISSSGGSIY
YPDSVKGRFTISRDNSKNTLYLQMNSLRAEDTAVYYCARDERWAGAMDAWGQGTTVTV
SSGGGGSGGGGSGGGGSDIQMTQSPSSLSASVGDRVTITCKASQNINKNLDWYQQKPGKA
PKLLIYETNNLQTGVPSRFSGSGSGTDFTLTISSLQPEDFATYYCYQHNSRFTFGGGTKLEI
KRLEPKSCDKTHTCPPCPAPEFQGGPSVFLFPPKPKDTLYITREPEVTCVVVDVSHEDPEVK
FNWYVDGVEVHNAKTKPREEQYNSTYRVVSVLTVLHQDWLNGKEYKCQVSNKALPAPIE
KTISKAKGQPREPQVYVYPPSRDELTKNQVSLTCLVKGFYPSDIAVEWESNGQPENNYKTT
PPVLDSDGSFALVSKLTVDKSRWQQGNVFSCSVMHEALHNHYTQKSLSLSPGEVQLVESG
GGLVQPGGSLRLSCAASGFTFNNYWMTWVRQAPGKGLEWVASISSSGGSIYYPDSVKGR
FTISRDNSKNTLYLQMNSLRAEDTAVYYCARDERWAGAMDAWGQGTTVTVSSGGGGSG
GGGSGGGGSDIQMTQSPSSLSASVGDRVTITCKASQNINKNLDWYQQKPGKAPKLLIYET
NNLQTGVPSRFSGSGSGTDFTLTISSLQPEDFATYYCYQHNSRFTFGGGTKLEIKRLE.

In some embodiments, the amino acid sequence of the anti-CD45 portion (scFv-Fc-scFv) of an anti-CD45×SLAMF6 tetravalent bispecific antibody comprises SEQ ID NO: 1 immediately followed by SEQ ID NO: 12. In some embodiments, the signal peptide is cleaved during post-translational modifications that occur in vitro or in vivo.

In some embodiments, the amino acid sequence of the anti-CD45 portion (scFv-Fc-scFv) of the anti-CD45×SLAMF6 tetravalent bispecific antibody comprises a signal peptide comprising SEQ ID NO: 1. MGWSCIILFLVATATGVHS (SEQ ID NO: 1). In some embodiments, the amino acid sequence of the anti-CD45 portion (scFv-Fc-scFv) of an anti-CD45×SLAMF6 tetravalent bispecific antibody (without the signal peptide) comprises SEQ ID NO: 22.

(SEQ ID NO: 22)
EVQLQQSGPELEKPGASVKISCKASGYSFTGYNMNWVKQSNGKSLEWIGNIDPYYGGSD
YSQKFLGKATLTVDKSSSTAYMHLKSLTSEDSAVYYCARSNMYDGEYYVMDYWGQGTS
VTVSSGGGGSGGGGSGGGGSDIVLTQSPASLAVSLGQRATISCRASESVDNFGVSFMNWF
QQKPGQPPKLLIYASSNRGSGVPARFSGSGSGTDFSLNVHPMEEDDAAMYFCQQSKAVPR
TFGGGTKLEIKPKSCDKTHTCPPCPAPEFQGGPSVFLFPPKPKDTLYITREPEVTCVVVDVSH
EDPEVKFNWYVDGVEVHNAKTKPREEQYNSTYRVVSVLTVLHQDWLNGKEYKCQVSNK
ALPAPIEKTISKAKGQPREPQVYVYPPSRDELTKNQVSLTCLVKGFYPSDIAVEWESNGQPE
NNYKTTPPVLDSDGSFALVSKLTVDKSRWQQGNVFSCSVMHEALHNHYTQKSLSLSPGEV
QLQQSGPELEKPGASVKISCKASGYSFTGYNMNWVKQSNGKSLEWIGNIDPYYGGSDYS
QKFLGKATLTVDKSSSTAYMHLKSLTSEDSAVYYCARSNMYDGEYYVMDYWGQGTSVT
VSSGGGGSGGGGSGGGGSDIVLTQSPASLAVSLGQRATISCRASESVDNFGVSFMNWFQQ
KPGQPPKLLIYASSNRGSGVPARFSGSGSGTDFSLNVHPMEEDDAAMYFCQQSKAVPRTF
GGGTKLEIK.

In some embodiments, the amino acid sequence of the anti-CD45 portion (scFv-Fc-scFv) of an anti-CD45×SLAMF6 tetravalent bispecific antibody comprises SEQ ID NO: 1 immediately followed by SEQ ID NO: 22. In some embodiments, the signal peptide is cleaved during post-translational modifications that occur in vitro or in vivo.

In some embodiments, the amino acid sequence of the anti-CD45 portion (scFv-Fc-scFv) of the anti-CD45×SLAMF6 tetravalent bispecific antibody comprises a signal peptide comprising SEQ ID NO: 1. In some embodiments, the amino acid sequence of the anti-CD45 portion (scFv-Fc-scFv) of an anti-CD45×SLAMF6 tetravalent bispecific antibody (without the signal peptide) comprises SEQ ID NO: 23.

(SEQ ID NO: 23)
DVQLVESGGGLVQPGGSRKLSCAASGFTFSNFGMHWVRQAPEKGLEWVAYVSRGSTTF
YYADTVKGRFTISRDNPKNTLFLQMTSLRSEDTAMYYCARSATATWTMDYWGHGTSVT
VSSGGGGSGGGGSGGGGSNIMMTQSPSSLAVSAGEKVTMSCKSSQSVFSSSNHMNYLAW
YQQKPGQSPKLLIYWASTRESGVPDRFTGSGSGTDFTLTISSVQAEDLAVYYCHQYLSSW
TFGGGSKLEIKPKSCDKTHTCPPCPAPEFQGGPSVFLFPPKPKDTLYITREPEVTCVVVDVSH
EDPEVKFNWYVDGVEVHNAKTKPREEQYNSTYRVVSVLTVLHQDWLNGKEYKCQVSNK
ALPAPIEKTISKAKGQPREPQVYVYPPSRDELTKNQVSLTCLVKGFYPSDIAVEWESNGQPE
NNYKTTPPVLDSDGSFALVSKLTVDKSRWQQGNVFSCSVMHEALHNHYTQKSLSLSPGDV
QLVESGGGLVQPGGSRKLSCAASGFTFSNFGMHWVRQAPEKGLEWVAYVSRGSTTFYY
ADTVKGRFTISRDNPKNTLFLQMTSLRSEDTAMYYCARSATATWTMDYWGHGTSVTVSS
GGGGSGGGGSGGGGSNIMMTQSPSSLAVSAGEKVTMSCKSSQSVESSSNHMNYLAWYQQ
KPGQSPKLLIYWASTRESGVPDRFTGSGSGTDFTLTISSVQAEDLAVYYCHQYLSSWTFGG
GSKLEIK.

In some embodiments, the amino acid sequence of the anti-CD45 portion (scFv-Fc-scFv) of an anti-CD45×SLAMF6 tetravalent bispecific antibody comprises SEQ ID NO: 1 immediately followed by SEQ ID NO: 23. In some embodiments, the signal peptide is cleaved during post-translational modifications that occur in vitro or in vivo.

In some embodiments, the amino acid sequence of the anti-CD45 portion (scFv-Fc-scFv) of the anti-CD45×SLAMF6 tetravalent bispecific antibody comprises a signal peptide comprising SEQ ID NO: 1. In some embodiments, the amino acid sequence of the anti-CD45 portion (scFv-Fc-scFv) of an anti-CD45×SLAMF6 tetravalent bispecific antibody (without the signal peptide) comprises SEQ ID NO: 24.

(SEQ ID NO: 24)
QVQLQQSAPELARPGASVRMSCKASGYTFTTYTMNWLTQRPGQGLEWIGYINPSSGYTE
YNQKFKDKTSLTADTSSSTAYMQLSSLTSEHSAVYYCARASGYSSWFAYWGQGTLVTVS
AGGGGSGGGGSGGGGSNIVMTQTPKFLLVSAGDRVTITCKASQSVNNDVGWYQQKPGQS
PKLLIYYASNRYTGVPDRFTGSGYGTDFTFTISTVQAEDLAVYFCQQDYNSPLTFGAGTKL
ELKPKSCDKTHTCPPCPAPEFQGGPSVFLFPPKPKDTLYITREPEVTCVVVDVSHEDPEVKF
NWYVDGVEVHNAKTKPREEQYNSTYRVVSVLTVLHQDWLNGKEYKCQVSNKALPAPIEK
TISKAKGQPREPQVYVYPPSRDELTKNQVSLTCLVKGFYPSDIAVEWESNGQPENNYKTTPP
VLDSDGSFALVSKLTVDKSRWQQGNVFSCSVMHEALHNHYTQKSLSLSPGQVQLQQSAPE
LARPGASVRMSCKASGYTFTTYTMNWLTQRPGQGLEWIGYINPSSGYTEYNQKFKDKTS
LTADTSSSTAYMQLSSLTSEHSAVYYCARASGYSSWFAYWGQGTLVTVSAGGGGSGGGG
SGGGGSNIVMTQTPKFLLVSAGDRVTITCKASQSVNNDVGWYQQKPGQSPKLLIYYASNR
YTGVPDRFTGSGYGTDFTFTISTVQAEDLAVYFCQQDYNSPLTFGAGTKLELK.

In some embodiments, the amino acid sequence of the anti-CD45 portion (scFv-Fc-scFv) of an anti-CD45×SLAMF6 tetravalent bispecific antibody comprises SEQ ID NO: 1 immediately followed by SEQ ID NO: 24. In some embodiments, the signal peptide is cleaved during post-translational modifications that occur in vitro or in vivo.

In some embodiments, the amino acid sequence of the anti-CD45 portion (scFv-Fc-scFv) of the anti-CD45×SLAMF6 tetravalent bispecific antibody comprises a signal peptide comprising SEQ ID NO: 1. In some embodiments, the amino acid sequence of the anti-CD45 portion (scFv-Fc-scFv) of an anti-CD45×SLAMF6 tetravalent bispecific antibody (without the signal peptide) comprises SEQ ID NO: 25.

(SEQ ID NO: 25)
QVQLQQSGAELARPGASVKMSCKASGYTFTFYAILWVKQMPGQGLEWIGFINPSSGYTSY
NQKFKDKATLTADKSSSTAYMQLSSLTSEDSAVYYCARDYGLDYWGQGTTLAVSSGGGG
SGGGGSGGGGSDIVMTQAAPSVPVTPGESVSISCRSSKSLLHSNGNTYLYWFLQRPGQSPQ
LLIYRMSNLASGVPDRFSGSGSGTAFTLGISRVEAGDVGVYYCMQHLEYPFTFGSGTKLEI
KPKSCDKTHTCPPCPAPEFQGGPSVFLFPPKPKDTLYITREPEVTCVVVDVSHEDPEVKFNW
YVDGVEVHNAKTKPREEQYNSTYRVVSVLTVLHQDWLNGKEYKCQVSNKALPAPIEKTIS
KAKGQPREPQVYVYPPSRDELTKNQVSLTCLVKGFYPSDIAVEWESNGQPENNYKTTPPVL
DSDGSFALVSKLTVDKSRWQQGNVFSCSVMHEALHNHYTQKSLSLSPGQVQLQQSGAELA
RPGASVKMSCKASGYTFTFYAILWVKQMPGQGLEWIGFINPSSGYTSYNQKFKDKATLT
ADKSSSTAYMQLSSLTSEDSAVYYCARDYGLDYWGQGTTLAVSSGGGGSGGGGSGGGGS
DIVMTQAAPSVPVTPGESVSISCRSSKSLLHSNGNTYLYWFLQRPGQSPQLLIYRMSNLAS
GVPDRFSGSGSGTAFTLGISRVEAGDVGVYYCMQHLEYPFT.

In some embodiments, the amino acid sequence of the anti-CD45 portion (scFv-Fc-scFv) of an anti-CD45×SLAMF6 tetravalent bispecific antibody comprises SEQ ID NO: 1 immediately followed by SEQ ID NO: 25. In some embodiments, the signal peptide is cleaved during post-translational modifications that occur in vitro or in vivo.

In some embodiments, the amino acid sequence of the anti-CD45 portion (scFv-Fc-scFv) of the anti-CD45×SLAMF6 tetravalent bispecific antibody comprises a signal peptide comprising SEQ ID NO: 1. In some embodiments, the amino acid sequence of the anti-CD45 portion (scFv-Fc-scFv) of an anti-CD45×SLAMF6 tetravalent bispecific antibody (without the signal peptide) comprises SEQ ID NO: 26.

(SEQ ID NO: 26)
DVQLVESGGGLVQPGGSRKLSCAASGFTFSSFGMHWVRQAPEKGLEWVAYINSGSTTFY
YADTVKGRFTISRDNPKNTLFLQMTSLGSEDTAMYYCARSATATWTMDYWGQGTSVTVS
SGGGGSGGGGSGGGGSNIMMTQSPSSLAVSAGEKVTMSCKSSQSVFVSSNQKNYLAWYQ
QKPGQSPKLLIYWASTRESAVPDRFTGSGSGTDFTLTIGSVQAEDLAVYYCHQYLSSWTFG
GGTKLEIKPKSCDKTHTCPPCPAPEFQGGPSVFLFPPKPKDTLYITREPEVTCVVVDVSHEDP
EVKFNWYVDGVEVHNAKTKPREEQYNSTYRVVSVLTVLHQDWLNGKEYKCQVSNKALP
APIEKTISKAKGQPREPQVYVYPPSRDELTKNQVSLTCLVKGFYPSDIAVEWESNGQPENNY
KTTPPVLDSDGSFALVSKLTVDKSRWQQGNVFSCSVMHEALHNHYTQKSLSLSPGDVQLV
ESGGGLVQPGGSRKLSCAASGFTFSSFGMHWVRQAPEKGLEWVAYINSGSTTFYYADTV
KGRFTISRDNPKNTLFLQMTSLGSEDTAMYYCARSATATWTMDYWGQGTSVTVSSGGGG
SGGGGSGGGGSNIMMTQSPSSLAVSAGEKVTMSCKSSQSVFVSSNQKNYLAWYQQKPGQ
SPKLLIYWASTRESAVPDRFTGSGSGTDFTLTIGSVQAEDLAVYYCHQYLSSWTFGGGTKL
EIK.

In some embodiments, the amino acid sequence of the anti-CD45 portion (scFv-Fc-scFv) of an anti-CD45×SLAMF6 tetravalent bispecific antibody comprises SEQ ID NO: 1 immediately followed by SEQ ID NO: 12. In some embodiments, the signal peptide is cleaved during post-translational modifications that occur in vitro or in vivo.

In some embodiments, the amino acid sequence of the anti-CD45 portion (scFv-Fc-scFv) of the anti-CD45×SLAMF6 tetravalent bispecific antibody comprises a signal peptide comprising SEQ ID NO: 1. In some embodiments, the amino acid sequence of the anti-CD45 portion (scFv-Fc-scFv) of an anti-CD45×SLAMF6 tetravalent bispecific antibody (without the signal peptide) comprises SEQ ID NO: 27.

(SEQ ID NO: 27)
EVQLQQSGAELVKPGASVKLSCTASGFNIKDTFMHWVKLRPEQGLEWIGRIDPANGYTK
YDPRFQGKATIIADTSSNTAYLQLSSLTSEDTAVYYCASGEYYALDYWGQGTSVTVSSGG
GGSGGGGSGGGGSDIVLTQSPASLTVSLGQRATISCRASKSVSTSGYSYMHWYQQKPGQP
PKLLIYLASNLESGVPARFSGSGSGTDFTLNIHPVEEEDAATYYCQHNRELPYTFGGGTKL
EIKPKSCDKTHTCPPCPAPEFQGGPSVFLFPPKPKDTLYITREPEVTCVVVDVSHEDPEVKFN
WYVDGVEVHNAKTKPREEQYNSTYRVVSVLTVLHQDWLNGKEYKCQVSNKALPAPIEKT
ISKAKGQPREPQVYVYPPSRDELTKNQVSLTCLVKGFYPSDIAVEWESNGQPENNYKTTPP
VLDSDGSFALVSKLTVDKSRWQQGNVFSCSVMHEALHNHYTQKSLSLSPGEVQLQQSGAE
LVKPGASVKLSCTASGFNIKDTFMHWVKLRPEQGLEWIGRIDPANGYTKYDPRFQGKATI
IADTSSNTAYLQLSSLTSEDTAVYYCASGEYYALDYWGQGTSVTVSSGGGGSGGGGSGGG
GSDIVLTQSPASLTVSLGQRATISCRASKSVSTSGYSYMHWYQQKPGQPPKLLIYLASNLE
SGVPARFSGSGSGTDFTLNIHPVEEEDAATYYCQHNRELPYTFGGGTKLEIK.

In some embodiments, the amino acid sequence of the anti-CD45 portion (scFv-Fc-scFv) of an anti-CD45×SLAMF6 tetravalent bispecific antibody comprises SEQ ID NO: 1 immediately followed by SEQ ID NO: 27. In some embodiments, the signal peptide is cleaved during post-translational modifications that occur in vitro or in vivo.

In some embodiments, the signal peptide of the anti-CD45 portion (scFv-Fc-scFv) of the anti-CD45×SLAMF6 tetravalent bispecific antibody comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-CD45 portion (scFv-Fc-scFv) of the anti-CD45×SLAMF6 tetravalent bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 12. In some embodiments, the anti-CD45 portion (scFv-Fc-scFv) of the anti-CD45×SLAMF6 tetravalent bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 22. In some embodiments, the anti-CD45 portion (scFv-Fc-scFv) of the anti-CD45×SLAMF6 tetravalent bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 23. In some embodiments, the anti-CD45 portion (scFv-Fc-scFv) of the anti-CD45×SLAMF6 tetravalent bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 24. In some embodiments, the anti-CD45 portion (scFv-Fc-scFv) of the anti-CD45×SLAMF6 tetravalent bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 25. In some embodiments, the anti-CD45 portion (scFv-Fc-scFv) of the anti-CD45×SLAMF6 tetravalent bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 26. In some embodiments, the anti-CD45 portion (scFv-Fc-scFv) of the anti-CD45×SLAMF6 tetravalent bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 27. In some embodiments, the anti-CD45 portion of an anti-CD45×SLAMF6 tetravalent bispecific antibody comprises an amino acid sequence comprising the CDRs of the variable heavy chain domains and the CDRs of the variable light chain domains of the anti-CD45 portion of the anti-CD45×SLAMF6 tetravalent bispecific antibody wherein the CDR sequences are indicated above (in bold and underline). In some embodiments, the framework regions (FRs) of the variable heavy chain domains and the FRs of the variable light chain domains of the anti-CD45 portion of the anti-CD45×SLAMF6 tetravalent bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 12. In some embodiments, the framework regions (FRs) of the variable heavy chain domains and the FRs of the variable light chain domains of the anti-CD45 portion of the anti-CD45×SLAMF6 tetravalent bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 22. In some embodiments, the framework regions (FRs) of the variable heavy chain domains and the FRs of the variable light chain domains of the anti-CD45 portion of the anti-CD45×SLAMF6 tetravalent bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 23. In some embodiments, the framework regions (FRs) of the variable heavy chain domains and the FRs of the variable light chain domains of the anti-CD45 portion of the anti-CD45×SLAMF6 tetravalent bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 24. In some embodiments, the framework regions (FRs) of the variable heavy chain domains and the FRs of the variable light chain domains of the anti-CD45 portion of the anti-CD45×SLAMF6 tetravalent bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 25. In some embodiments, the framework regions (FRs) of the variable heavy chain domains and the FRs of the variable light chain domains of the anti-CD45 portion of the anti-CD45×SLAMF6 tetravalent bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 26. In some embodiments, the framework regions (FRs) of the variable heavy chain domains and the FRs of the variable light chain domains of the anti-CD45 portion of the anti-CD45×SLAMF6 tetravalent bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 27.

Although the sequence above is depicted with linkers “GGGGSGGGGSGGGGS” (SEQ ID NO: 14) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker is between about 2 and 25 amino acids in length. In some embodiments, the glycine-serine linker is about 15 amino acids in length.

### Nucleic Acid Sequences of Anti-CD45 Portions of Anti-CD45×SLAMF6 Tetravalent Bispecific Antibodies

The amino acid sequences of the respective CDRs, linker portions, VH portion, VL portion, Fc portion, and knob/hole mutations are provided herein. Nucleic acid sequences encoding the respective CDRs, linker portions, VH portions, VL portions, Fc portion and knob/hole mutations can be determined from the amino acid sequence by a person of skill in the art. Nucleic acid sequences encoding the respective CDRs, linker portions, VH portion, VL portion, Fc portion and knob/hole mutations can be determined from the amino acid sequence of SEQ ID NO: 12 by a person of skill in the art. Nucleic acid sequences encoding the respective CDRs, linker portions, VH portion, VL portion, Fc portion and knob/hole mutations can be determined from the amino acid sequence of SEQ ID NO: 22 by a person of skill in the art. Nucleic acid sequences encoding the respective CDRs, linker portions, VH portion, VL portion, Fc portion and knob/hole mutations can be determined from the amino acid sequence of SEQ ID NO: 23 by a person of skill in the art. Nucleic acid sequences encoding the respective CDRs, linker portions, VH portion, VL portion, Fc portion and knob/hole mutations can be determined from the amino acid sequence of SEQ ID NO: 24 by a person of skill in the art. Nucleic acid sequences encoding the respective CDRs, linker portions, VH portion, VL portion, Fc portion and knob/hole mutations can be determined from the amino acid sequence of SEQ ID NO: 25 by a person of skill in the art. Nucleic acid sequences encoding the respective CDRs, linker portions, VH portion, VL portion, Fc portion and knob/hole mutations can be determined from the amino acid sequence of SEQ ID NO: 26 by a person of skill in the art. Nucleic acid sequences encoding the respective CDRs, linker portions, VH portion, VL portion, Fc portion and knob/hole mutations can be determined from the amino acid sequence of SEQ ID NO: 27 by a person of skill in the art. In some embodiments, instead of the “hole” mutations, the nucleic acid sequence comprising the anti-CD3 portion of an anti-CD3×SLAMF6 bispecific antibody can comprise a nucleic acid sequence encoding the “knob” mutations, while the “hole” mutations are present on an anti-SLAMF6 portion of an anti-CD3×SLAMF6 tetravalent bispecific antibody. In some embodiments, the nucleic acid sequences encoding the respective CDRs, linker portions, VH portions, VL portions, Fc portion and knob/hole mutations may be codon optimized.

### Amino Acid Sequences of Anti-CD43 Portions of Anti-CD43×SLAMF6 Tetravalent Bispecific Antibodies

In some embodiments, the anti-CD43 portion of the anti-CD43×SLAMF6 tetravalent bispecific antibody is a first single-chain variable fragment (scFv) with a fragment crystallizable (Fc) region and a second single-chain variable fragment (scFv) (i.e., scFv-Fc-scFv antibody) comprising a signal peptide, a first variable heavy chain (VH) domain, a first linker, a first variable light chain (VL) domain, an Fc portion, a second variable heavy chain (VH) domain, a second linker, and a second variable light chain (VL) domain. Underlined and bolded amino acids represent the respective complementarity determining regions (CDRs). Double underlined amino acids represent a linker sequence between the variable heavy chain and the variable light chain portions. Underlined amino acids represent the fragment, crystallizable (Fc) region. Bolded, underlined, and italicized amino acids represent the “hole” mutations for the production of knob-in-hole antibodies. In some embodiments, instead of the “hole” mutations, the amino acid sequence comprising the anti-CD43 portion of an anti-CD43×SLAMF6 tetravalent bispecific antibody can comprise the “knob” mutations, while the “hole” mutations are present on an anti-SLAMF6 portion of an anti-CD43×SLAMF6 tetravalent bispecific antibody.

In some embodiments, the amino acid sequence of the anti-CD43 portion (scFv-Fc-scFv) of the anti-CD43×SLAMF6 tetravalent bispecific antibody comprises a signal peptide comprising SEQ ID NO: 1. MGWSCIILFLVATATGVHS (SEQ ID NO: 1). In some embodiments, the amino acid sequence of the anti-CD43 portion (scFv-Fc-scFv) of an anti-CD43×SLAMF6 bispecific antibody (without the signal peptide) comprises SEQ ID NO: 13.

(SEQ ID NO: 13)
QVQLVQSGSELKKPGASVKVSCKASGYTFTNFGMNWVRQAPGQGLEWMGWINTYSGE
ATYADDFKGRFVFSLDTSVSTAYLQISSLKAEDTAVYYCARRGGTAEFDYWGQGTLVTV
SSGGGGSGGGGSGGGGSEIVLTQSPDFQSVTPKEKVTITCSASSSISSNFLHWYQQKPDQS
PKLLIYRTSKLASGVPSRFSGSGSGTDFTLTINSLEAEDAATYYCQQGIYMPLTFGGGTKL
EIKRLEPKSCDKTHTCPPCPAPEFQGGPSVFLFPPKPKDTLYITREPEVTCVVVDVSHEDPEV
KFNWYVDGVEVHNAKTKPREEQYNSTYRVVSVLTVLHQDWLNGKEYKCQVSNKALPAPI
EKTISKAKGQPREPQVYVLPPSRDELTKNQVSLLCLVKGFYPSDIAVEWESNGQPENNYLT
WPPVLDSDGSFFLYSKLTVDKSRWQQGNVFSCSVMHEALHNHYTQKSLSLSPGQVQLVQS
GSELKKPGASVKVSCKASGYTFTNFGMNWVRQAPGQGLEWMGWINTYSGEATYADDF
KGRFVFSLDTSVSTAYLQISSLKAEDTAVYYCARRGGTAEFDYWGQGTLVTVSSGGGGS
GGGGSGGGGSEIVLTQSPDFQSVTPKEKVTITCSASSSISSNFLHWYQQKPDQSPKLLIYRT
SKLASGVPSRFSGSGSGTDFTLTINSLEAEDAATYYCQQGIYMPLTFGGGTKLEIKRLE.

In some embodiments, the amino acid sequence of the anti-CD43 portion (scFv-Fc-scFv) of an anti-CD43×SLAMF6 tetravalent bispecific antibody comprises SEQ ID NO: 1 immediately followed by SEQ ID NO: 13. In some embodiments, the signal peptide is cleaved during post-translational modifications that occur in vitro or in vivo.

In some embodiments, the signal peptide of the anti-CD43 portion (scFv-Fc-scFv) of the anti-CD43×SLAMF6 tetravalent bispecific antibody comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-CD43 portion (scFv-Fc-scFv) of the anti-CD43×SLAMF6 tetravalent bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 13. In some embodiments, the anti-CD43 portion of an anti-CD43×SLAMF6 tetravalent bispecific antibody comprises an amino acid sequence comprising the CDRs of the variable heavy chain domain and the CDRs of the variable light chain domain of the anti-CD43 portion of the anti-CD43×SLAMF6 tetravalent bispecific antibody wherein the CDR sequences are indicated above (in bold and underline). In some embodiments, the framework regions (FRs) of the variable heavy chain domain and the FRs of the variable light chain domain of the anti-CD43 portion of the anti-CD43×SLAMF6 tetravalent bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 13.

Although the sequence above is depicted with linkers “GGGGSGGGGSGGGGS” (SEQ ID NO: 14) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker is between about 2 and 25 amino acids in length. In some embodiments, the glycine-serine linker is about 15 amino acids in length.

### Nucleic Acid Sequences of Anti-CD43 Portions of Anti-CD43×SLAMF6 Tetravalent Bispecific Antibodies

The amino acid sequences of the respective CDRs, linker portions, VH portion, VL portion, Fc portion, and knob/hole mutations are provided herein. Nucleic acid sequences encoding the respective CDRs, linker portions, VH portions, VL portions, Fc portion and knob/hole mutations can be determined from the amino acid sequence by a person of skill in the art. Nucleic acid sequences encoding the respective CDRs, linker portions, VH portion, VL portion, Fc portion and knob/hole mutations can be determined from the amino acid sequence of SEQ ID NO: 13 by a person of skill in the art. In some embodiments, instead of the “hole” mutations, the nucleic acid sequence comprising the anti-CD43 portion of an anti-CD43×SLAMF6 bispecific antibody can comprise a nucleic acid sequence encoding the “knob” mutations, while the “hole” mutations are present on an anti-SLAMF6 portion of an anti-CD43×SLAMF6 tetravalent bispecific antibody. In some embodiments, the nucleic acid sequences encoding the respective CDRs, linker portions, VH portions, VL portions, Fc portion and knob/hole mutations may be codon optimized.

### Amino Acid Sequences of Anti-SLAMF6 Portions of Anti-CD43×SLAMF6, Anti-CD45×SLAMF6, or Anti-CD43×SLAMF6 Tetravalent Bispecific Antibodies

In some embodiments, the anti-SLAMF6 portion of the anti-CD3×SLAMF6, anti-CD45×SLAMF6, or anti-CD43×SLAMF6 tetravalent bispecific antibody is a first single-chain variable fragment (scFv) with a fragment crystallizable (Fc) region and a second single-chain variable fragment (scFv) (i.e., scFv-Fc-scFv antibody) comprising a signal peptide, a first variable heavy chain (VH) domain, a first linker, a first variable light chain (VL) domain, an Fc portion, a second variable heavy chain (VH) domain, a second linker, and a second variable light chain (VL) domain. Underlined and bolded amino acids represent the respective complementarity determining regions (CDRs). Double underlined amino acids represent a linker sequence between the variable heavy chain and the variable light chain portions. Underlined amino acids represent the fragment, crystallizable (Fc) region. Bolded, underlined, and italicized amino acids represent the “hole” mutations for the production of knob-in-hole antibodies. In some embodiments, instead of the “hole” mutations, the amino acid sequence comprising the anti-SLAMF6 portion of an anti-CD3×SLAMF6, anti-CD45×SLAMF6, or anti-CD43×SLAMF6 tetravalent bispecific antibody can comprise the “knob” mutations, while the “hole” mutations are present on an anti-CD3×SLAMF6 portion, and anti-CD45×SLAMF6 portion, or an anti-CD43×SLAMF6 portion of an anti-CD3×SLAMF6, anti-CD45×SLAMF6, or anti-CD43×SLAMF6 tetravalent bispecific antibody.

In some embodiments, the amino acid sequence of the anti-SLAMF6 portion (scFv-Fc-scFv) of the anti-CD3×SLAMF6, anti-CD45×SLAMF6, or anti-CD43×SLAMF6 tetravalent bispecific antibody comprises a signal peptide comprising SEQ ID NO: 1. MGWSCIILFLVATATGVHS (SEQ ID NO: 1). In some embodiments, the amino acid sequence of the anti-SLAMF6 portion (scFv-Fc-scFv) of an anti-CD43×SLAMF6 bispecific antibody (without the signal peptide) comprises SEQ ID NO: 16.

(SEQ ID NO: 16).
QVQLVQSGSELKKPGASVKVSCKASGYTFTNFGMNWVRQAPGQGLEWMGWINTYSGE
ATYADDFKGRFVFSLDTSVSTAYLQISSLKAEDTAVYYCARRGGTAEFDYWGQGTLVTV
SSGGGGSGGGGSGGGGSEIVLTQSPDFQSVTPKEKVTITCSASSSISSNFLHWYQQKPDQSP
KLLIYRTSKLASGVPSRFSGSGSGTDFTLTINSLEAEDAATYYCQQGIYMPLTFGGGTKLEI
KRLEPKSCDKTHTCPPCPAPEFQGGPSVFLFPPKPKDTLYITREPEVTCVVVDVSHEDPEVK
FNWYVDGVEVHNAKTKPREEQYNSTYRVVSVLTVLHQDWLNGKEYKCQVSNKALPAPIE
KTISKAKGQPREPQVYVLPPSRDELTKNQVSLLCLVKGFYPSDIAVEWESNGQPENNYLTW
PPVLDSDGSFFLYSKLTVDKSRWQQGNVFSCSVMHEALHNHYTQKSLSLSPGQVQLVQSG
SELKKPGASVKVSCKASGYTFTNFGMNWVRQAPGQGLEWMGWINTYSGEATYADDFK
GRFVFSLDTSVSTAYLQISSLKAEDTAVYYCARRGGTAEFDYWGQGTLVTVSSGGGGSG
GGGSGGGGSEIVLT.

In some embodiments, the amino acid sequence of the anti-SLAMF6 portion (scFv-Fc-scFv) of an anti-CD3×SLAMF6, anti-CD45×SLAMF6, or anti-CD43×SLAMF6 tetravalent bispecific antibody comprises SEQ ID NO: 1 immediately followed by SEQ ID NO: 16. In some embodiments, the signal peptide is cleaved during post-translational modifications that occur in vitro or in vivo.

In some embodiments, the signal peptide of the anti-SLAMF6 portion (scFv-Fc-scFv) of the anti-CD3×SLAMF6, anti-CD45×SLAMF6, or anti-CD43×SLAMF6 tetravalent bispecific antibody comprises an amino acid sequence 80, 81, 82, 83, 84, 85, 86, 87, 88, 89, 90, 91, 92, 93, 94, 95, 96, 97, 98 or 99% identical to SEQ ID NO: 1. In some embodiments, the anti-CDSLAMF6 portion (scFv-Fc-scFv) of the anti-CD3×SLAMF6, anti-CD45×SLAMF6, or anti-CD43×SLAMF6 tetravalent bispecific antibody (without the signal peptide) comprises an amino acid sequence 80, 81, 82, 83, 84, 85, 86, 87, 88, 89, 90, 91, 92, 93, 94, 95, 96, 97, 98 or 99% identical to SEQ ID NO: 16. In some embodiments, the anti-SLAMF6 portion of an anti- anti-CD3×SLAMF6, anti-CD45×SLAMF6, or anti-CD43×SLAMF6 tetravalent bispecific antibody comprises an amino acid sequence comprising the CDRs of the variable heavy chain domain and the CDRs of the variable light chain domain of the anti-SLAMF6 portion of the anti-CD3×SLAMF6, anti-CD45×SLAMF6, or anti-CD43×SLAMF6 tetravalent bispecific antibody wherein the CDR sequences are indicated above (in bold and underline). In some embodiments, the framework regions (FRs) of the variable heavy chain domain and the FRs of the variable light chain domain of the anti-CD43 portion of the anti-CD3×SLAMF6, anti-CD45×SLAMF6, or anti-CD43×SLAMF6 tetravalent bispecific antibody comprise an amino acid sequence 80, 81, 82, 83, 84, 85, 86, 87, 88, 89, 90, 91, 92, 93, 94, 95, 96, 97, 98 or 99% identical to the FRs of SEQ ID NO: 16.

Although the sequence above is depicted with linkers “GGGGSGGGGSGGGGS” (SEQ ID NO: 14) a person of skill in the art recognizes that numerous other flexible linkers known in the art can be used, including those enriched in small or hydrophilic amino acids. In some embodiments, the linker between the VH and VL domains comprises a glycine-serine linker. In some embodiments, any combination of glycine and serine residues can be used. In some embodiments, the glycine-serine linker is between about 2 and 25 amino acids in length. In some embodiments, the glycine-serine linker is about 15 amino acids in length.

### Nucleic Acid Sequences of Anti-SLAMF6 Portions of Anti-CD3×SLAMF6, Anti-CD45×SLAMF6, or Anti-CD43×SLAMF6 Tetravalent Bispecific Antibodies

The amino acid sequences of the respective CDRs, linker portions, VH portion, VL portion, Fc portion, and knob/hole mutations are provided herein. Nucleic acid sequences encoding the respective CDRs, linker portions, VH portions, VL portions, Fc portion and knob/hole mutations can be determined from the amino acid sequence by a person of skill in the art. Nucleic acid sequences encoding the respective CDRs, linker portions, VH portion, VL portion, Fc portion and knob/hole mutations can be determined from the amino acid sequence of SEQ ID NO: 16 by a person of skill in the art. In some embodiments, instead of the “knob” mutations, the nucleic acid sequence comprising the anti-SLAMF6 portion of an anti-CD3×SLAMF6, anti-CD45×SLAMF6, or anti-CD43×SLAMF6 tetravalent bispecific antibody can comprise a nucleic acid sequence encoding the “hole” mutations, while the “knob” mutations are present on an anti-CD3 portion, and anti-CD45 portion, or an anti-CD43 portion of an anti-CD3×SLAMF6, anti-CD45×SLAMF6, or anti-CD43×SLAMF6 tetravalent bispecific antibody. In some embodiments, the nucleic acid sequences encoding the respective CDRs, linker portions, VH portions, VL portions, Fc portion and knob/hole mutations may be codon optimized.

### Amino Acid Sequences of Anti-CD3×SLAMF6 Bispecific Antibodies

The antibody comprising SEQ ID NO: 2 of the anti-CD3 portion and SEQ ID NO: 9 of the anti-SLAMF6 portion is represented by antibody “anti-CD3×SLAMF6” in embodiments described and depicted in this disclosure.

The antibody comprising SEQ ID NO: 11 of the anti-CD3 portion and SEQ ID NO: 16 of the anti-SLAMF6 portion is represented by antibody “anti-CD3×SLAMF6 tetravalent” in embodiments described and depicted in this disclosure.

### Nucleic Acid Sequences of Anti-CD3×SLAMF6 Bispecific Antibodies

The antibody encoded by a nucleic acid sequence comprising SEQ ID NO: 4 of the anti-CD3 portion and SEQ ID NO: 10 of the anti-SLAMF6 portion is represented by antibody “anti-CD3×SLAMF6” in embodiments described and depicted in this disclosure.

### Amino Acid Sequences of Anti-CD45×SLAMF6 Bispecific Antibodies

The antibody comprising SEQ ID NO: 5 of the anti-CD45 portion and SEQ ID NO: 9 of the anti-SLAMF6 portion is represented by antibody “anti-CD45×SLAMF6” in embodiments described and depicted in this disclosure.

The antibody comprising SEQ ID NO: 12 of the anti-CD45 portion and SEQ ID NO: 16 of the anti-SLAMF6 portion is represented by antibody “anti-CD45×SLAMF6 tetravalent” in embodiments described and depicted in this disclosure.

The antibody comprising SEQ ID NO: 28 of the anti-CD45 portion and SEQ ID NO: 9 of the anti-SLAMF6 portion is represented by antibody “anti-CD45×SLAMF6” in embodiments described and depicted in this disclosure. The anti-CD45 portion corresponds to the variable domains of anti-CD45 antibody clone 23.

The antibody comprising SEQ ID NO: 29 of the anti-CD45 portion and SEQ ID NO: 9 of the anti-SLAMF6 portion is represented by antibody “anti-CD45×SLAMF6” in embodiments described and depicted in this disclosure. The anti-CD45 portion corresponds to the variable domains of anti-CD45 antibody clone 26.

The antibody comprising SEQ ID NO: 30 of the anti-CD45 portion and SEQ ID NO: 9 of the anti-SLAMF6 portion is represented by antibody “anti-CD45×SLAMF6” in embodiments described and depicted in this disclosure. The anti-CD45 portion corresponds to the variable domains of anti-CD45 antibody clone 27.

The antibody comprising SEQ ID NO: 31 of the anti-CD45 portion and SEQ ID NO: 9 of the anti-SLAMF6 portion is represented by antibody “anti-CD45×SLAMF6” in embodiments described and depicted in this disclosure. The anti-CD45 portion corresponds to the variable domains of anti-CD45 antibody clone 28.

The antibody comprising SEQ ID NO: 32 of the anti-CD45 portion and SEQ ID NO: 9 of the anti-SLAMF6 portion is represented by antibody “anti-CD45×SLAMF6” in embodiments described and depicted in this disclosure. The anti-CD45 portion corresponds to the variable domains of anti-CD45 antibody clone 31.

The antibody comprising SEQ ID NO: 33 of the anti-CD45 portion and SEQ ID NO: 9 of the anti-SLAMF6 portion is represented by antibody “anti-CD45×SLAMF6” in embodiments described and depicted in this disclosure. The anti-CD45 portion corresponds to the variable domains of anti-CD45 antibody clone 42.

The antibody comprising SEQ ID NO: 22 of the anti-CD45 portion and SEQ ID NO: 16 of the anti-SLAMF6 portion is represented by antibody “anti-CD45×SLAMF6 tetravalent” in embodiments described and depicted in this disclosure. The anti-CD45 portion corresponds to the variable domains of anti-CD45 antibody clone 23.

The antibody comprising SEQ ID NO: 23 of the anti-CD45 portion and SEQ ID NO: 16 of the anti-SLAMF6 portion is represented by antibody “anti-CD45×SLAMF6 tetravalent” in embodiments described and depicted in this disclosure. The anti-CD45 portion corresponds to the variable domains of anti-CD45 antibody clone 26.

The antibody comprising SEQ ID NO: 24 of the anti-CD45 portion and SEQ ID NO: 16 of the anti-SLAMF6 portion is represented by antibody “anti-CD45×SLAMF6 tetravalent” in embodiments described and depicted in this disclosure. The anti-CD45 portion corresponds to the variable domains of anti-CD45 antibody clone 27.

The antibody comprising SEQ ID NO: 25 of the anti-CD45 portion and SEQ ID NO: 16 of the anti-SLAMF6 portion is represented by antibody “anti-CD45×SLAMF6 tetravalent” in embodiments described and depicted in this disclosure. The anti-CD45 portion corresponds to the variable domains of anti-CD45 antibody clone 28.

The antibody comprising SEQ ID NO: 26 of the anti-CD45 portion and SEQ ID NO: 16 of the anti-SLAMF6 portion is represented by antibody “anti-CD45×SLAMF6 tetravalent” in embodiments described and depicted in this disclosure. The anti-CD45 portion corresponds to the variable domains of anti-CD45 antibody clone 31.

The antibody comprising SEQ ID NO: 27 of the anti-CD45 portion and SEQ ID NO: 16 of the anti-SLAMF6 portion is represented by antibody “anti-CD45×SLAMF6 tetravalent” in embodiments described and depicted in this disclosure. The anti-CD45 portion corresponds to the variable domains of anti-CD45 antibody clone 42.

### Nucleic Acid Sequences of Anti-CD45×SLAMF6 Bispecific Antibodies

The antibody encoded by a nucleic acid sequence comprising SEQ ID NO: 6 of the anti-CD45 portion and SEQ ID NO: 10 of the anti-SLAMF6 portion is represented by antibody “anti-CD45×SLAMF6” in embodiments described and depicted in this disclosure.

The antibody encoded by a nucleic acid sequence comprising SEQ ID NO: 34 of the anti-CD45 portion and SEQ ID NO: 10 of the anti-SLAMF6 portion is represented by antibody “anti-CD45×SLAMF6” in embodiments described and depicted in this disclosure. The anti-CD45 portion corresponds to the variable domains of anti-CD45 antibody clone 23.

The antibody encoded by a nucleic acid sequence comprising SEQ ID NO: 35 of the anti-CD45 portion and SEQ ID NO: 10 of the anti-SLAMF6 portion is represented by antibody “anti-CD45×SLAMF6” in embodiments described and depicted in this disclosure. The anti-CD45 portion corresponds to the variable domains of anti-CD45 antibody clone 26.

The antibody encoded by a nucleic acid sequence comprising SEQ ID NO: 36 of the anti-CD45 portion and SEQ ID NO: 10 of the anti-SLAMF6 portion is represented by antibody “anti-CD45×SLAMF6” in embodiments described and depicted in this disclosure. The anti-CD45 portion corresponds to the variable domains of anti-CD45 antibody clone 27.

The antibody encoded by a nucleic acid sequence comprising SEQ ID NO: 37 of the anti-CD45 portion and SEQ ID NO: 10 of the anti-SLAMF6 portion is represented by antibody “anti-CD45×SLAMF6” in embodiments described and depicted in this disclosure. The anti-CD45 portion corresponds to the variable domains of anti-CD45 antibody clone 28.

The antibody encoded by a nucleic acid sequence comprising SEQ ID NO: 38 of the anti-CD45 portion and SEQ ID NO: 10 of the anti-SLAMF6 portion is represented by antibody “anti-CD45×SLAMF6” in embodiments described and depicted in this disclosure. The anti-CD45 portion corresponds to the variable domains of anti-CD45 antibody clone 31.

The antibody encoded by a nucleic acid sequence comprising SEQ ID NO: 39 of the anti-CD45 portion and SEQ ID NO: 10 of the anti-SLAMF6 portion is represented by antibody “anti-CD45×SLAMF6” in embodiments described and depicted in this disclosure. The anti-CD45 portion corresponds to the variable domains of anti-CD45 antibody clone 42.

### Amino Acid Sequences of Anti-CD43×SLAMF6 Bispecific Antibodies

The antibody comprising SEQ ID NO: 7 of the anti-CD43 portion and SEQ ID NO: 9 of the anti-SLAMF6 portion is represented by antibody “anti-CD43×SLAMF6” in embodiments described and depicted in this disclosure.

The antibody comprising SEQ ID NO: 13 of the anti-CD43 portion and SEQ ID NO: 16 of the anti-SLAMF6 portion is represented by antibody “anti-CD43×SLAMF6 tetravalent” in embodiments described and depicted in this disclosure.

### Nucleic Acid Sequences of Anti-CD43×SLAMF6 Bispecific Antibodies

The antibody encoded by a nucleic acid sequence comprising SEQ ID NO: 8 of the anti-CD43 portion and SEQ ID NO: 10 of the anti-SLAMF6 portion is represented by antibody “anti-CD43×SLAMF6” in embodiments described and depicted in this disclosure.

### Structure of AntiCD3×SLAMF6, Anti-CD45×SLAMF6, and Anti-CD43×SLAMF6 Antibodies

In some embodiments, the molecular three-dimensional structure of an anti-CD3×SLAMF6, an anti-CD45×SLAMF6, or an anti-CD43×SLAMF6 bispecific antibody can be predicted based on X-ray crystallography, and/or cryo-EM, and/or using structure prediction algorithms (e.g., machine learning algorithms) known in the art, such as AlphaFold or RaptorX. In some embodiments, the structure prediction algorithm is a computational method that is used to predict three-dimensional (3D) antibody structures based on a given nucleic acid or amino acid sequence. In some embodiments, the structure prediction algorithm predicts the 3D coordinates of all heavy atoms for a given antibody using a nucleic acid or amino acid sequence and/or aligned sequences of homologues as inputs. In some embodiments, the structure of an anti-CD3×SLAMF6, an anti-CD45×SLAMF6, or an anti-CD43×SLAMF6 bispecific antibody is predicted using a combination of methods, e.g., using a combination of AlphaFold (or any other structure prediction algorithm known in the art) and X-ray crystallography or cryo-EM. In some embodiments, the structure prediction is improved by combining the use of AlphaFold (or any other structure prediction algorithm known in the art) and X-ray crystallography or cryo-EM. In some embodiments, the structure of an anti-CD3×SLAMF6, an anti-CD45×SLAMF6, or an anti-CD43×SLAMF6 bispecific antibody is predicted by using a computational structure prediction algorithm (e.g., AlphaFold or RaptorX) and the structure prediction of the anti-CD3×SLAMF6, the anti-CD45×SLAMF6, or the anti-CD43×SLAMF6 bispecific antibody is then refined by using X-ray crystallography or cryo-EM. In some embodiments, the anti-CD3×SLAMF6, the anti-CD45×SLAMF6, or the anti-CD43×SLAMF6 bispecific antibody comprises a three-dimensional structure that is similar to the three-dimensional structure of an anti-CD3×SLAMF6, an anti-CD45×SLAMF6, or an anti-CD43×SLAMF6 bispecific antibody that comprises SEQ ID NO: 2, 5, 7, 9, 11, 12, 13, 16, 28, 29, 30, 31, 32, 33, 22, 23, 24, 25, 26, 27, or a combination thereof.

In some embodiments, the structure prediction algorithm can be used to model the structure of a first bispecific antibody (e.g., an anti-CD3×SLAMF6, an anti-CD45×SLAMF6, or an anti-CD43×SLAMF6 bispecific antibody) (e.g., a reference an CD3×SLAMF6, an anti-CD45×SLAMF6, or an anti-CD43×SLAMF6 bispecific antibody comprising SEQ ID NO: 2, 5, 7, 9, 11, 12, 13, 16, 28, 29, 30, 31, 32, 33, 22, 23, 24, 25, 26, 27, or a combination thereof) and compare a predicted structure of second bispecific antibody (e.g., an CD3×SLAMF6, an anti-CD45×SLAMF6, or an anti-CD43×SLAMF6 bispecific antibody) against the predicted structure of the first antibody such that the second antibody can be categorized in the same class as the first bispecific antibody based on its structural similarity to the first bispecific antibody. In some embodiments, a metric of structural similarity between two antibodies can be obtained based on the output of a structure prediction algorithm known in the art. In some embodiments, the metric of structural similarity between two antibodies is based on a similarity distance.

In some embodiments, the structure of the anti-CD3×SLAMF6 bispecific antibody allows the anti-CD3×SLAMF6 bispecific antibody to bind to CD3 and SLAMF6. In some embodiments, disclosed herein is a new class of anti-CD3×SLAMF6 bispecific antibodies which comprise structural similarity to one another such that the new class of anti-CD3×SLAMF6 bispecific antibodies are capable of binding to CD3 and SLAMF6. In some embodiments, the anti-CD3×SLAMF6 bispecific antibody comprises means for binding CD3 and SLAMF6. In some embodiments, means for binding CD3 and SLAMF6 comprises an anti-CD3×SLAMF6 bispecific antibody that comprises SEQ ID NOs: 2 and 9.

In some embodiments, the structure of the anti-CD45×SLAMF6 bispecific antibody allows the anti-CD45×SLAMF6 bispecific antibody to bind to CD45 and SLAMF6. In some embodiments, disclosed herein is a new class of anti-CD45×SLAMF6 bispecific antibodies which comprise structural similarity to one another such that the new class of anti-CD45×SLAMF6 bispecific antibodies are capable of binding to CD45 and SLAMF6. In some embodiments, the anti-CD45×SLAMF6 bispecific antibody comprises means for binding CD45 and SLAMF6. In some embodiments, means for binding CD45 and SLAMF6 comprises an anti-CD45×SLAMF6 bispecific antibody that comprises SEQ ID NOs: 5 and 9. In some embodiments, means for binding CD45 and SLAMF6 comprises an anti-CD45×SLAMF6 bispecific antibody that comprises SEQ ID NOs: 28 and 9. In some embodiments, means for binding CD45 and SLAMF6 comprises an anti-CD45×SLAMF6 bispecific antibody that comprises SEQ ID NOs: 29 and 9. In some embodiments, means for binding CD45 and SLAMF6 comprises an anti-CD45×SLAMF6 bispecific antibody that comprises SEQ ID NOs: 30 and 9. In some embodiments, means for binding CD45 and SLAMF6 comprises an anti-CD45×SLAMF6 bispecific antibody that comprises SEQ ID NOs: 31 and 9. In some embodiments, means for binding CD45 and SLAMF6 comprises an anti-CD45×SLAMF6 bispecific antibody that comprises SEQ ID NOs: 32 and 9. In some embodiments, means for binding CD45 and SLAMF6 comprises an anti-CD45×SLAMF6 bispecific antibody that comprises SEQ ID NOs: 33 and 9.

In some embodiments, the structure of the anti-CD43×SLAMF6 bispecific antibody allows the anti-CD43×SLAMF6 bispecific antibody to bind to CD43 and SLAMF6. In some embodiments, disclosed herein is a new class of anti-CD43×SLAMF6 bispecific antibodies which comprise structural similarity to one another such that the new class of anti-CD43×SLAMF6 bispecific antibodies are capable of binding to CD43 and SLAMF6. In some embodiments, the anti-CD43×SLAMF6 bispecific antibody comprises means for binding CD43 and SLAMF6. In some embodiments, means for binding CD43 and SLAMF6 comprises an anti-CD43×SLAMF6 bispecific antibody that comprises SEQ ID NO: 7 and 9.

### Non-limiting Embodiments of the Subject Matter

In some embodiments, the anti-CD3×SLAMF6, the anti-CD45×SLAMF6, or the anti-CD43×SLAMF6 antibody disclosed herein can be used as an immunotherapy for cancer treatment, as a research model for studying the immune synapse dynamics and T cell function, for treatment of an autoimmune disease, and/or for the development of cancer vaccines by incorporating the bispecific antibody to stimulate targeted and robust T-cell responses against tumor-specific antigens.

In certain aspects, described herein is a bispecific antibody or a fragment thereof, comprising: a first arm comprising a first variable heavy chain domain and a first variable light chain domain, wherein a portion of the first arm is capable of binding to a portion of a CD3 protein, a portion of a CD45, portion, or a portion of a CD43 protein; and a second arm comprising a second variable heavy chain domain and a second variable light chain domain, wherein a portion of the second arm is capable of binding to a portion of a SLAMF6 protein.

In some embodiments, the first arm is encoded by a first polypeptide chain and the second arm is encoded by a second polypeptide chain that associate together. In some embodiments, the first arm comprises a linker between the first variable heavy domain and first variable light chain domain. In some embodiments, the second arm comprises a linker between the first variable heavy domain and first variable light chain domain. In some embodiments, the linker is a glycine-serine linker.

In some embodiments, the first and second arms each further comprise a fragment, crystallizable (Fc) region. In some embodiments, the Fc region of the first arm comprises knob mutations and the Fc region of the second arm comprise hole mutations, or vice versa.

In some embodiments, the bispecific antibody is bivalent. In some embodiments, the first arm and second arm are encoded on a first polypeptide chain.

In some embodiments, the first polypeptide chain further comprises: a third arm comprising a third variable heavy chain domain and a third variable light chain domain that is the same as the first variable heavy chain domain and first variable light chain domain; and the second polypeptide chain further comprises: a fourth arm comprising a fourth variable heavy chain domain and a fourth variable light chain domain, that is the same as the second variable heavy chain domain and second variable light chain domain.

In some embodiments, the first arm comprises a linker between the first variable heavy domain and first variable light chain domain. In some embodiments, the second arm comprises a linker between the first variable heavy domain and first variable light chain domain. In some embodiments, the third arm comprises a linker between the third variable heavy domain and third variable light chain domain. In some embodiments, the fourth arm comprises a linker between the fourth variable heavy domain and fourth variable light chain domain. In some embodiments, the linker is a glycine-serine linker.

In some embodiments, the first and second polypeptide chains each further comprises a fragment, crystallizable (Fc) region. In some embodiments, the Fc region of the first polypeptide chain comprises knob mutations and the Fc region of the second polypeptide chain comprises hole mutations, or vice versa. In some embodiments, the Fc region of the first polypeptide chain is positioned between the first arm and third arm and the Fc region of the second polypeptide chain is positioned between the second arm and fourth arm. In some embodiments, the bispecific antibody is tetravalent.

In some embodiments, the first variable heavy chain domain comprises an amino acid sequence of the variable heavy chain portion of SEQ ID NO: 2, wherein the first variable light chain domain comprises an amino acid sequence of the variable light chain portion of SEQ ID NO: 2. In some embodiments, the first arm comprises SEQ ID NO: 2, wherein the second arm comprises SEQ ID NO: 9. In some embodiments, the first variable heavy chain domain comprises an amino acid sequence of the variable heavy chain portion of SEQ ID NO: 5, wherein the first variable light chain domain comprises an amino acid sequence of the variable light chain portion of SEQ ID NO: 5. In some embodiments, the first arm comprises SEQ ID NO: 5, wherein the second arm comprises SEQ ID NO: 9. In some embodiments, the first variable heavy chain domain comprises an amino acid sequence of the variable heavy chain portion of SEQ ID NO: 28, wherein the first variable light chain domain comprises an amino acid sequence of the variable light chain portion of SEQ ID NO: 28. In some embodiments, the first arm comprises SEQ ID NO: 28, wherein the second arm comprises SEQ ID NO: 9. In some embodiments, the first variable heavy chain domain comprises an amino acid sequence of the variable heavy chain portion of SEQ ID NO: 29, wherein the first variable light chain domain comprises an amino acid sequence of the variable light chain portion of SEQ ID NO: 29. In some embodiments, the first arm comprises SEQ ID NO: 29, wherein the second arm comprises SEQ ID NO: 9. In some embodiments, the first variable heavy chain domain comprises an amino acid sequence of the variable heavy chain portion of SEQ ID NO: 30, wherein the first variable light chain domain comprises an amino acid sequence of the variable light chain portion of SEQ ID NO: 30. In some embodiments, the first arm comprises SEQ ID NO: 30, wherein the second arm comprises SEQ ID NO: 9. In some embodiments, the first variable heavy chain domain comprises an amino acid sequence of the variable heavy chain portion of SEQ ID NO: 31, wherein the first variable light chain domain comprises an amino acid sequence of the variable light chain portion of SEQ ID NO: 31. In some embodiments, the first arm comprises SEQ ID NO: 31, wherein the second arm comprises SEQ ID NO: 9. In some embodiments, the first variable heavy chain domain comprises an amino acid sequence of the variable heavy chain portion of SEQ ID NO: 32, wherein the first variable light chain domain comprises an amino acid sequence of the variable light chain portion of SEQ ID NO: 32. In some embodiments, the first arm comprises SEQ ID NO: 32, wherein the second arm comprises SEQ ID NO: 9. In some embodiments, the first variable heavy chain domain comprises an amino acid sequence of the variable heavy chain portion of SEQ ID NO: 33, wherein the first variable light chain domain comprises an amino acid sequence of the variable light chain portion of SEQ ID NO: 33. In some embodiments, the first arm comprises SEQ ID NO: 33, wherein the second arm comprises SEQ ID NO: 9.

In some embodiments, the first variable heavy chain domain comprises an amino acid sequence of the variable heavy chain portion of SEQ ID NO: 7, wherein the first variable light chain domain comprises an amino acid sequence of the variable light chain portion of SEQ ID NO: 7. In some embodiments, the first arm comprises SEQ ID NO: 7, wherein the second arm comprises SEQ ID NO: 9.

In some embodiments, the second variable heavy chain domain comprises an amino acid sequence of the variable heavy chain portion of SEQ ID NO: 9, wherein the first variable light chain domain comprises an amino acid sequence of the variable light chain portion of SEQ ID NO: 9. In some embodiments, the first arm comprises an amino acid sequence selected from the group consisting of SEQ ID NO: 2, SEQ ID NO: 5, SEQ ID NO: 7, SEQ ID NO: 28, SEQ ID NO: 29, SEQ ID NO: 30, SEQ ID NO: 31, SEQ ID NO: 32, or SEQ ID NO: 33 and wherein the second arm comprises SEQ ID NO: 9.

In some embodiments, the portion of the first arm is capable of binding to the portion of the CD3 protein, and wherein the bispecific antibody is capable of clustering a SLAMF6 protein with a core of an immune synapse. In some embodiments, the bispecific antibody is capable of promoting downstream signaling of a SLAMF6 mediated response in a T cell. In some embodiments, the bispecific antibody is capable of enhancing T cell function. In some embodiments, the bispecific antibody enhancement of T cell function is greater than either monospecific antibody does individually or in a nonspecific combination mixture. In some embodiments, the bispecific antibody enhancement of T cell function is dose-dependent. In some embodiments, the bispecific antibody is capable of binding to the portion of the CD3 protein and to the portion of the SLAMF6 protein, wherein the CD3 protein and SLAMF6 protein are located on a same T cell.

In some embodiments, the portion of the first arm is capable of binding to the portion of the CD45 protein, and wherein the bispecific antibody is capable of localizing a SLAMF6 protein away from a core of an immune synapse.

In some embodiments, the bispecific antibody is capable of disrupting downstream signaling of inhibitory proteins and effector pathways in a T cell. In some embodiments, the bispecific antibody is capable of enhancing T cell function. In some embodiments, the bispecific antibody enhancement of T cell function is greater than either monospecific antibody does individually or in a nonspecific combination mixture. In some embodiments, the bispecific antibody enhancement of T cell function is dose-dependent.

In some embodiments, the bispecific antibody is capable of binding to the portion of the CD45 protein and to the portion of the SLAMF6 protein, wherein the CD45 protein and SLAMF6 protein are located on a same T cell. In some embodiments, the portion of the first arm is capable of binding to the portion of the CD43 protein, and wherein the bispecific antibody is capable of localizing a SLAMF6 protein away from a core of an immune synapse. In some embodiments, the bispecific antibody is capable of disrupting a downstream signaling of inhibitory proteins and effector pathways in a T cell. In some embodiments, the bispecific antibody is capable of enhancing T cell function. In some embodiments, the bispecific antibody enhancement of T cell function is greater than either monospecific antibody does individually or in a nonspecific combination mixture. In some embodiments, the bispecific antibody enhancement of T cell function is dose-dependent. In some embodiments, the bispecific antibody is capable of binding to the portion of the CD43 protein and to the portion of the SLAMF6 protein, wherein the CD43 protein and SLAMF6 protein are located on a same T cell.

In some embodiments, the SLAMF6 protein is located on a T cell, and wherein the bispecific antibody is capable of promoting binding of a SLAM associated protein (SAP) to an intracellular tail of the SLAMF6 protein on a tumor cell. In some embodiments, the bispecific antibody is capable of inducing a cytokine secretion in a T cell. In some embodiments, the cytokine secretion is a secretion of IL-2.

In certain aspects, described herein is a tetravalent bispecific antibody or a fragment thereof, comprising: (I) a first fragment antigen-binding (Fab) region, comprising: a first arm comprising a first variable heavy chain domain and a first variable light chain domain, wherein a portion of the first arm is capable of binding to a portion of a CD3 protein, a portion of a CD45 protein, or a portion of a CD43 protein; and a second arm comprising a second variable heavy chain domain and a second variable light chain domain, wherein a portion of the second arm is capable of binding to a portion of a CD3 protein, a portion of a CD45 protein, or a portion of a CD43 protein; (II) a first fragment, crystallizable (Fc) region; (III) a second Fab region, comprising: a third arm comprising a first variable heavy chain domain and a first variable light chain domain, wherein a portion of the first arm is capable of binding to a portion of a SLAMF6 protein; and a fourth arm comprising a second variable heavy chain domain and a second variable light chain domain, wherein a portion of the second arm is capable of binding to a portion of a SLAMF6 protein; and (IV) a second Fc region, wherein the first and second Fc regions associated together.

In some embodiments, one arm of the first Fab region is N-terminal to the first Fc region and the other arm of the first Fab region is C-terminal to the first Fc region, and the first arm of the second Fab region is N-terminal to the second Fc region and the other arm of the second Fab region is C-terminal to the second Fc region. In some embodiments, one arm of the first Fab region is C-terminal to the first Fc region and one arm of the second Fab region is N-terminal to the first Fc region, and the other arm of the first Fab region is C-terminal to the second Fc region and the other arm of the second Fab region is N-terminal to the second Fc region, or one arm of the first Fab region is N-terminal to the first Fc region and one arm of the second Fab region is C-terminal to the first Fc region, and the other arm of the first Fab region is N-terminal to the second Fc region and the other arm of the second Fab region is C-terminal to the second Fc region.

In certain aspects, described herein is a pharmaceutical composition comprising: the bispecific antibody or the tetravalent bispecific antibody of any of the embodiments as described above herein; and a pharmaceutically acceptable carrier.

In certain aspects, described herein is a method of preventing or treating cancer in a subject comprising administering to the subject an effective amount of the pharmaceutical composition as described above herein. In some embodiments, the cancer is selected from colorectal cancer, lung cancer, bladder cancer, breast cancer, cervical cancer, kidney cancer, leukemia, Hodgkin lymphoma, non-Hodgkin lymphoma, prostate cancer, skin cancer (e.g., melanoma), head and neck cancer, endometrial cancer, colon cancer, rectal cancer, liver cancer, thyroids cancer, esophageal cancer, renal cell cancer, and a combination thereof.

In certain aspects, described above herein is a method of preventing or treating an autoimmune disease in a subject comprising administering to the subject an effective amount of the pharmaceutical composition as described above herein. In some embodiments, the autoimmune disease is Systemic lupus erythematosus (Lupus).

In some embodiments, the enhancement of T-cell activation is indicated by IFN-γ levels. In some embodiments, the enhancement of T-cell activation is indicated by phosphorylation of CD3 zeta.

In certain aspects, described above herein is a kit for generating a bispecific antibody or fragment thereof, the kit comprising one or more vectors comprising a polynucleotide sequence encoding any of the bispecific antibodies as described above herein.

In certain aspects, described above herein is a kit for generating a tetravalent bispecific antibody or fragment thereof, the kit comprising one or more vectors comprising a polynucleotide sequence encoding any of the tetravalent bispecific antibodies as described above herein.

In certain aspects, described above herein is a kit for generating a bispecific antibody or fragment thereof, the kit comprising: a first vector comprising a polynucleotide sequence encoding a first arm of the bispecific antibody or fragment thereof, wherein a portion of the first arm is capable of binding to a portion of a CD3, protein, a portion of a CD45 protein, or a portion of a CD43 protein; and a second vector comprising a polynucleotide sequence encoding a second arm of the bispecific antibody or fragment thereof, wherein a portion of the second arm is capable of binding to a portion of a SLAMF6 protein.

In some embodiments, the first vector and the second vector are the same vector. In some embodiments, the first vector and the second vector are two different vectors. In some embodiments, a variable heavy chain domain of the first arm comprises an amino acid sequence of the variable heavy chain portion of SEQ ID NO: 2, 5, 7, 28, 29, 30, 31, 32, or 33, a first variable light chain domain of the first arm comprises an amino acid sequence of the variable light chain portion of SEQ ID NO: 2, 5, 7, 28, 29, 30, 31, 32, or 33, a variable heavy chain domain of the second arm comprises an amino acid sequence of the variable heavy chain portion of SEQ ID NO: 9, and a variable light chain domain of the second arm comprises an amino acid sequence of the variable light chain portion of SEQ ID NO: 9.

In some embodiments, the first arm comprises an amino acid sequence selected from the group consisting of SEQ ID NO: 2, SEQ ID NO: 5, SEQ ID NO: 7, SEQ ID NO: 28, SEQ ID NO: 29, SEQ ID NO: 30, SEQ ID NO: 31, SEQ ID NO: 32, and SEQ ID NO: 33, wherein the second arm comprises SEQ ID NO: 9. In some embodiments, the first arm comprises SEQ ID NO: 2. In some embodiments, the first arm comprises SEQ ID NO: 5. In some embodiments, the first arm comprises SEQ ID NO: 7. In some embodiments, the first arm comprises SEQ ID NO: 28. In some embodiments, the first arm comprises SEQ ID NO: 29. In some embodiments, the first arm comprises SEQ ID NO: 30. In some embodiments, the first arm comprises SEQ ID NO: 31. In some embodiments, the first arm comprises SEQ ID NO: 32. In some embodiments, the first arm comprises SEQ ID NO: 33.

In some embodiments, the kit for generating a tetravalent bispecific antibody or fragment thereof comprises one or more vectors comprising a polynucleotide sequence encoding an amino acid sequence of SEQ ID NO: 11, SEQ ID NO: 12, SEQ ID NO: 13, SEQ ID NO: 22, SEQ ID NO: 23, SEQ ID NO: 24, SEQ ID NO: 25, SEQ ID NO: 26, or SEQ ID NO: 27 and a polynucleotide sequence encoding an amino acid sequence of SEQ ID NO: 16.

In some embodiments, disclosed herein is one or more host cells comprising one or more vectors comprising a polynucleotide sequence encoding an amino acid sequence of SEQ ID NO: 11, SEQ ID NO: 12, SEQ ID NO: 13, SEQ ID NO: 22, SEQ ID NO: 23, SEQ ID NO: 24, SEQ ID NO: 25, SEQ ID NO: 26, or SEQ ID NO: 27 and a polynucleotide sequence encoding an amino acid sequence of SEQ ID NO: 16.

In some embodiments, disclosed herein is a composition comprising one or more vectors comprising a polynucleotide sequence encoding an amino acid sequence of SEQ ID NO: 11, SEQ ID NO: 12, SEQ ID NO: 13, SEQ ID NO: 22, SEQ ID NO: 23, SEQ ID NO: 24, SEQ ID NO: 25, SEQ ID NO: 26, or SEQ ID NO: 27 and a polynucleotide sequence encoding an amino acid sequence of SEQ ID NO: 16.

In certain aspects, described above herein is one or more host cells comprising: one or more vectors comprising a polynucleotide sequence encoding any of the bispecific antibodies or any of the tetravalent bispecific antibodies as described above herein.

In certain aspects, described above herein is one or more host cells comprising: a first vector comprising a polynucleotide sequence encoding a first arm of a bispecific antibody or fragment thereof, wherein a portion of the first arm is capable of binding to a portion of a CD3 protein, a portion of a CD45 protein, or a portion of a CD43 protein; and a second vector comprising a polynucleotide sequence encoding a second arm of the bispecific antibody or fragment thereof, wherein a portion of the second arm is capable of binding to a portion of a SLAMF6 protein.

In some embodiments, the first vector and the second vector are the same vector. In some embodiments, the first vector and the second vector are two different vectors. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 2, and the first variable light chain domain comprises an amino acid sequence of the variable light chain portion of SEQ ID NO: 2. In some embodiments, the first arm comprises SEQ ID NO: 2, wherein the second arm comprises SEQ ID NO: 9.

In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 5, and the first variable light chain domain comprises an amino acid sequence of the variable light chain portion of SEQ ID NO: 5. In some embodiments, the first arm comprises SEQ ID NO: 5, and the second arm comprises SEQ ID NO: 9.

In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 7, and the first variable light chain domain comprises an amino acid sequence of the variable light chain portion of SEQ ID NO: 7. In some embodiments, the first arm comprises SEQ ID NO: 7, and the second arm comprises SEQ ID NO: 9.

In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 28, and the first variable light chain domain comprises an amino acid sequence of the variable light chain portion of SEQ ID NO: 28. In some embodiments, the first arm comprises SEQ ID NO: 28, and the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 29, and the first variable light chain domain comprises an amino acid sequence of the variable light chain portion of SEQ ID NO: 29. In some embodiments, the first arm comprises SEQ ID NO: 29, and the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 30, and the first variable light chain domain comprises an amino acid sequence of the variable light chain portion of SEQ ID NO: 30. In some embodiments, the first arm comprises SEQ ID NO: 30, and the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 31, and the first variable light chain domain comprises an amino acid sequence of the variable light chain portion of SEQ ID NO: 31. In some embodiments, the first arm comprises SEQ ID NO: 31, and the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 32, and the first variable light chain domain comprises an amino acid sequence of the variable light chain portion of SEQ ID NO: 32. In some embodiments, the first arm comprises SEQ ID NO: 32, and the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 33, and the first variable light chain domain comprises an amino acid sequence of the variable light chain portion of SEQ ID NO: 33. In some embodiments, the first arm comprises SEQ ID NO: 10, and the second arm comprises SEQ ID NO: 9.

In certain aspects, described above herein is a method of making a bispecific antibody or fragment thereof comprising: culturing the one or more host cells as described above herein under conditions suitable for an expression of the one or more vectors; and recovering the bispecific antibody or fragment thereof or tetravalent bispecific antibody or fragment thereof.

In certain aspects, described above herein is a method of making a bispecific antibody or fragment thereof comprising: culturing the one or more host cells as described above herein under conditions suitable for an expression of the first vector and the second vector; and recovering the bispecific antibody or fragment thereof.

In certain aspects, described above herein is a composition comprising: one or more vectors comprising a polynucleotide sequence encoding any of the bispecific antibodies or any of the tetravalent bispecific antibodies as described above herein.

In certain aspects, described above herein is a composition comprising: a first vector comprising a polynucleotide sequence encoding a first arm of the bispecific antibody or fragment thereof, wherein a portion of the first arm is capable of binding to a portion of a CD3 protein, a portion of a CD45 protein, or a portion of a CD43 protein; and a second vector comprising a polynucleotide sequence encoding a second arm of the bispecific antibody or fragment thereof, wherein a portion of the second arm is capable of binding to a portion of a SLAMF6 protein.

In some embodiments, the first vector and the second vector are the same vector. In some embodiments, the first vector and the second vector are two different vectors. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 2, and the first variable light chain domain comprises an amino acid sequence of the variable light chain portion of SEQ ID NO: 2. In some embodiments, the first arm comprises SEQ ID NO: 2, and the second arm comprises SEQ ID NO: 9.

In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 5, and the first variable light chain domain comprises an amino acid sequence of the variable light chain portion of SEQ ID NO: 5. In some embodiments, the first arm comprises SEQ ID NO: 5, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 7, and the first variable light chain domain comprises an amino acid sequence of the variable light chain portion of SEQ ID NO: 7. In some embodiments, the first arm comprises SEQ ID NO: 7, and the second arm comprises SEQ ID NO: 9.

In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 28, and the first variable light chain domain comprises an amino acid sequence of the variable light chain portion of SEQ ID NO: 28. In some embodiments, the first arm comprises SEQ ID NO: 28, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 29, and the first variable light chain domain comprises an amino acid sequence of the variable light chain portion of SEQ ID NO: 29. In some embodiments, the first arm comprises SEQ ID NO: 29, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 30, and the first variable light chain domain comprises an amino acid sequence of the variable light chain portion of SEQ ID NO: 30. In some embodiments, the first arm comprises SEQ ID NO: 30, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 31, and the first variable light chain domain comprises an amino acid sequence of the variable light chain portion of SEQ ID NO: 31. In some embodiments, the first arm comprises SEQ ID NO: 31, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 32, and the first variable light chain domain comprises an amino acid sequence of the variable light chain portion of SEQ ID NO: 32. In some embodiments, the first arm comprises SEQ ID NO: 32, wherein the second arm comprises SEQ ID NO: 9. In some embodiments, the first arm comprises a first variable heavy chain domain and a first variable light chain domain, the second arm comprises a second variable heavy chain domain and a second variable light chain domain, the first variable heavy chain domain comprises an amino acid sequence of the variable heavy chain portion of SEQ ID NO: 33, and the first variable light chain domain comprises an amino acid sequence of the variable light chain portion of SEQ ID NO: 33. In some embodiments, the first arm comprises SEQ ID NO: 33, wherein the second arm comprises SEQ ID NO: 9.

In certain aspects, described above herein is a means for binding: a portion of a CD3 protein, a portion of a CD45 protein, or a portion of a CD43 protein; and a portion of a SLAMF6 protein. In some embodiments, the means comprises a bispecific antibody or fragment thereof.

In some embodiments, the bispecific antibody or a fragment thereof comprises: a first arm comprising a first variable heavy chain domain and a first variable light chain domain, wherein a portion of the first arm is capable of binding to the portion of the CD3 protein, the portion of the CD45 protein, or the portion of the CD43 protein; and a second arm comprising a second variable heavy chain domain and a second variable light chain domain, wherein a portion of the second arm is capable of binding to the portion of the SLAMF6 protein. In some embodiments, the first variable heavy chain domain comprises an amino acid sequence of the variable heavy chain portion of SEQ ID NO: 2, the first variable light chain domain comprises an amino acid sequence of the variable light chain portion of SEQ ID NO: 2, and the portion of the first arm is capable of binding to the portion of the CD3 protein. In some embodiments, the first arm comprises SEQ ID NO: 2, the second arm comprises SEQ ID NO: 9, and the portion of the first arm is capable of binding to the portion of the CD3 protein.

In some embodiments, the first variable heavy chain domain comprises an amino acid sequence of the variable heavy chain portion of SEQ ID NO: 5, the first variable light chain domain comprises an amino acid sequence of the variable light chain portion of SEQ ID NO: 5, and the portion of the first arm is capable of binding to the portion of the CD45 protein. In some embodiments, the first arm comprises SEQ ID NO: 5, the second arm comprises SEQ ID NO: 9, and the portion of the first arm is capable of binding to the portion of the CD45 protein.

In some embodiments, the first variable heavy chain domain comprises an amino acid sequence of the variable heavy chain portion of SEQ ID NO: 7, the first variable light chain domain comprises an amino acid sequence of the variable light chain portion of SEQ ID NO: 7, and the portion of the first arm is capable of binding to the portion of the CD43 protein. In some embodiments, the first arm comprises SEQ ID NO: 7, wherein the second arm comprises SEQ ID NO: 9, and wherein the portion of the second arm is capable of binding to the portion of the CD43 protein.

In some embodiments, the first variable heavy chain domain comprises an amino acid sequence of the variable heavy chain portion of SEQ ID NO: 28, the first variable light chain domain comprises an amino acid sequence of the variable light chain portion of SEQ ID NO: 28, and the portion of the first arm is capable of binding to the portion of the CD45 protein. In some embodiments, the first arm comprises SEQ ID NO: 28, the second arm comprises SEQ ID NO: 9, and the portion of the first arm is capable of binding to the portion of the CD45 protein. In some embodiments, the first variable heavy chain domain comprises an amino acid sequence of the variable heavy chain portion of SEQ ID NO: 29, the first variable light chain domain comprises an amino acid sequence of the variable light chain portion of SEQ ID NO: 29, and the portion of the first arm is capable of binding to the portion of the CD45 protein. In some embodiments, the first arm comprises SEQ ID NO: 29, the second arm comprises SEQ ID NO: 9, and the portion of the first arm is capable of binding to the portion of the CD45 protein. In some embodiments, the first variable heavy chain domain comprises an amino acid sequence of the variable heavy chain portion of SEQ ID NO: 30, the first variable light chain domain comprises an amino acid sequence of the variable light chain portion of SEQ ID NO: 30, and the portion of the first arm is capable of binding to the portion of the CD45 protein. In some embodiments, the first arm comprises SEQ ID NO: 30, the second arm comprises SEQ ID NO: 9, and the portion of the first arm is capable of binding to the portion of the CD45 protein. In some embodiments, the first variable heavy chain domain comprises an amino acid sequence of the variable heavy chain portion of SEQ ID NO: 31, the first variable light chain domain comprises an amino acid sequence of the variable light chain portion of SEQ ID NO: 31, and the portion of the first arm is capable of binding to the portion of the CD45 protein. In some embodiments, the first arm comprises SEQ ID NO: 31, the second arm comprises SEQ ID NO: 9, and the portion of the first arm is capable of binding to the portion of the CD45 protein. In some embodiments, the first variable heavy chain domain comprises an amino acid sequence of the variable heavy chain portion of SEQ ID NO: 32, the first variable light chain domain comprises an amino acid sequence of the variable light chain portion of SEQ ID NO: 32, and the portion of the first arm is capable of binding to the portion of the CD45 protein. In some embodiments, the first arm comprises SEQ ID NO: 32, the second arm comprises SEQ ID NO: 9, and the portion of the first arm is capable of binding to the portion of the CD45 protein. In some embodiments, the first variable heavy chain domain comprises an amino acid sequence of the variable heavy chain portion of SEQ ID NO: 33, the first variable light chain domain comprises an amino acid sequence of the variable light chain portion of SEQ ID NO: 33, and the portion of the first arm is capable of binding to the portion of the CD45 protein. In some embodiments, the first arm comprises SEQ ID NO: 33, the second arm comprises SEQ ID NO: 9, and the portion of the first arm is capable of binding to the portion of the CD45 protein.

In some embodiments, the portion of the first arm is capable of binding to the portion of the CD3 protein, and wherein the bispecific antibody is capable of clustering a SLAMF6 protein with a core of an immune synapse. In some embodiments, the bispecific antibody is capable of promoting downstream signaling of a SLAMF6 mediated response in a T cell. In some embodiments, the bispecific antibody is capable of enhancing T cell function. In some embodiments, the bispecific antibody is capable of binding to the portion of the CD3 protein and to the portion on the SLAMF6 protein, wherein the CD3 protein and SLAMF6 protein are located on a same T cell.

In some embodiments, the portion of the first arm is capable of binding to the portion of the CD45 protein, and the bispecific antibody is capable of localizing a SLAMF6 protein away from a core of an immune synapse. In some embodiments, the bispecific antibody is capable of disrupting downstream signaling of inhibitory proteins and effector pathways in a T cell. In some embodiments, the bispecific antibody is capable of enhancing T cell function.

In some embodiments, the bispecific antibody is capable of binding to the portion of the CD45 protein and to the portion on the SLAMF6 protein, wherein the CD45 protein and SLAMF6 protein are located on a same T cell.

In some embodiments, the portion of the first arm is capable of binding to the portion of the CD43 protein, and wherein the bispecific antibody is capable of localizing a SLAMF6 protein away from a core of an immune synapse. In some embodiments, the bispecific antibody is capable of disrupting a downstream signaling of inhibitory proteins and effector pathways in a T cell. In some embodiments, the bispecific antibody is capable of enhancing T cell function. In some embodiments, the bispecific antibody is capable of binding to the portion of the CD43 protein and to the portion on the SLAMF6 protein wherein the CD43 protein and SLAMF6 protein are located on a same T cell.

In some embodiments, the SLAMF6 protein is located on a T cell, and wherein the bispecific antibody is capable of promoting binding of a SLAM associated protein (SAP) to an intracellular tail of the SLAMF6 protein on a tumor cell. In some embodiments, the bispecific antibody is capable of inducing a cytokine secretion in a T cell. In some embodiments, the cytokine secretion is a secretion of IL-2.

In some embodiments, the bispecific antibody or a fragment thereof comprises: a third arm comprising a first variable heavy chain domain and a first variable light chain domain, wherein a portion of the first arm is capable of binding to the portion of the CD3 protein, the portion of the CD45 protein, or the portion of the CD43 protein; and a fourth arm comprising a second variable heavy chain domain and a second variable light chain domain, wherein a portion of the second arm is capable of binding to the portion of the SLAMF6 protein. In some embodiments, the bispecific antibody comprises SEQ ID NO: 11 and SEQ ID NO: 16. In some embodiments, the bispecific antibody comprises SEQ ID NO: 12 and SEQ ID NO: 16. In some embodiments, the bispecific antibody comprises SEQ ID NO: 13 and SEQ ID NO: 16. In some embodiments, the bispecific antibody comprises SEQ ID NO: 22 and SEQ ID NO: 16. In some embodiments, the bispecific antibody comprises SEQ ID NO: 23 and SEQ ID NO: 16. In some embodiments, the bispecific antibody comprises SEQ ID NO: 24 and SEQ ID NO: 16. In some embodiments, the bispecific antibody comprises SEQ ID NO: 25 and SEQ ID NO: 16. In some embodiments, the bispecific antibody comprises SEQ ID NO: 26 and SEQ ID NO: 16. In some embodiments, the bispecific antibody comprises SEQ ID NO: 27 and SEQ ID NO: 16.

### Compositions

In some embodiments, a prophylactic or therapeutic composition of this disclosure comprises one or more antibodies (or one or more polynucleotides encoding one or more antibodies) and is administered in a pharmaceutical composition that includes a pharmaceutically acceptable carrier. In some embodiments, the prophylactic or therapeutic composition is comprised of one or more antibodies (or one or more polynucleotides encoding one or more antibodies) comprising SEQ ID NOs: 2 and 9 (e.g., antibody “anti-CD3×SLAMF6”), SEQ ID NOs: 5 and 9, (e.g., “anti-CD45SLAMF6”), SEQ ID NOs: 28 and 9, (e.g., “anti-CD45×SLAMF6”), SEQ ID NOs: 29 and 9, (e.g., “anti-CD45×SLAMF6”), SEQ ID NOs: 30 and 9, (e.g., “anti-CD45×SLAMF6”), SEQ ID NOs: 31 and 9, (e.g., “anti-CD45×SLAMF6”), SEQ ID NOs: 32 and 9, (e.g., “anti-CD45×SLAMF6”), SEQ ID NOs: 33 and 9, (e.g., “anti-CD45×SLAMF6”), SEQ ID NOs: 22 and 16, (e.g., “anti-CD45×SLAMF6 tetravalent”), SEQ ID NOs: 23 and 16, (e.g., “anti-CD45×SLAMF6 tetravalent”), SEQ ID NOs: 24 and 16, (e.g., “anti-CD45SLAMF6 tetravalent”), SEQ ID NOs: 25 and 16, (e.g., “anti-CD45×SLAMF6 tetravalent”), SEQ ID NOs: 26 and 16, (e.g., “anti-CD45×SLAMF6 tetravalent”), or SEQ ID NOs: 27 and 16, (e.g., “anti-CD45×SLAMF6 tetravalent”), SEQ ID NOs: 7 and 9 (“anti-CD43SLAMF6”), SEQ ID NOs: 11 and 16 (e.g., antibody “anti-CD3×SLAMF6 tetravalent”), SEQ ID NOs: 12 and 16, (e.g., “anti-CD45SLAMF6 tetravalent”), or comprising SEQ ID NOs: 13 and 16 (“anti-CD43SLAMF6 tetravalent”). In some embodiments, the pharmaceutical composition is in the form of a spray, aerosol, gel, solution, emulsion, nanoparticle (e.g., lipid nanoparticle), or suspension.

The composition is preferably administered to a subject with a pharmaceutically acceptable carrier. Typically, in some embodiments, an appropriate amount of a pharmaceutically acceptable salt is used in the formulation, which in some embodiments can render the formulation isotonic.

In certain embodiments, the one or more antibodies (or one or more polynucleotides encoding one or more antibodies) are provided as a composition comprising any one of the antibodies described herein (e.g., “anti-CD3×SLAMF6”, “anti-CD45×SLAMF6”, anti-CD43×SLAMF6”, “anti-CD3×SLAMF6 tetravalent”, “anti-CD45×SLAMF6 tetravalent”, anti-CD43×SLAMF6 tetravalent” bispecific antibody) and a pharmaceutically acceptable carrier. In certain embodiments, the composition further comprises an adjuvant. In certain embodiments, the antibodies are conjugated with other molecules to increase their effectiveness as is known by those practiced in the art.

In some embodiments, the pharmaceutically acceptable carrier is selected from the group consisting of saline, Ringer's solution, dextrose solution, and a combination thereof. Other suitable pharmaceutically acceptable carriers known in the art are contemplated. Suitable carriers and their formulations are described in Remington's Pharmaceutical Sciences, 2005, Mack Publishing Co. The pH of the solution is preferably from about 5 to about 8, and more preferably from about 7 to about 7.5. The formulation may also comprise a lyophilized powder. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of antibodies being administered.

The phrase pharmaceutically acceptable carrier as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier is acceptable in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term carrier denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency. The composition may also include additional agents such as an isotonicity agent, a preservative, a surfactant, and, a divalent cation, preferably, zinc.

The composition can also include an excipient, or an agent for stabilization of an antibody composition, such as a buffer, a reducing agent, a bulk protein, amino acids (such as e.g., glycine or praline) or a carbohydrate. Typical carbohydrates useful in formulating compositions include but are not limited to sucrose, mannitol, lactose, trehalose, or glucose.

Surfactants may also be used to prevent soluble and insoluble aggregation and/or precipitation of antibodies included in the composition. Suitable surfactants include but are not limited to sorbitan trioleate, soya lecithin, and oleic acid. In certain cases, solution aerosols are preferred using solvents such as ethanol. Thus, formulations including antibodies can also include a surfactant that can reduce or prevent surface-induced aggregation of antibodies by atomization of the solution in forming an aerosol. Various conventional surfactants can be employed, such as polyoxyethylene fatty acid esters and alcohols, and polyoxyethylene sorbitol fatty acid esters. Amounts will generally range between 0.001% and 4% by weight of the formulation. In some embodiments, surfactants used with the present disclosure are polyoxyethylene sorbitan mono-oleate, polysorbate 80, polysorbate 20. Additional agents known in the art can also be included in the composition.

In some embodiments, the pharmaceutical compositions and dosage forms further comprise one or more compounds that reduce the rate by which an active ingredient will decay, or the composition will change in character. So called stabilizers or preservatives may include, but are not limited to, amino acids, antioxidants, pH buffers, or salt buffers. Nonlimiting examples of antioxidants include butylated hydroxy anisole (BHA), ascorbic acid and derivatives thereof, tocopherol and derivatives thereof, butylated hydroxy anisole and cysteine. Nonlimiting examples of preservatives include parabens, such as methyl or propyl p-hydroxybenzoate and benzalkonium chloride. Additional nonlimiting examples of amino acids include glycine or proline.

The present invention also teaches the stabilization (preventing or minimizing thermally or mechanically induced soluble or insoluble aggregation and/or precipitation of an inhibitor protein) of liquid solutions containing antibodies at neutral pH or less than neutral pH by the use of amino acids including proline or glycine, with or without divalent cations resulting in clear or nearly clear solutions that are stable at room temperature or preferred for pharmaceutical administration.

In one embodiment, the composition is a pharmaceutical composition of single unit or multiple unit dosage forms. Pharmaceutical compositions of single unit or multiple unit dosage forms of the invention comprise a prophylactically or therapeutically effective amount of one or more compositions (e.g., a compound of the invention, or other prophylactic or therapeutic agent), typically, one or more vehicles, carriers, or excipients, stabilizing agents, and/or preservatives. Preferably, the vehicles, carriers, excipients, stabilizing agents and preservatives are pharmaceutically acceptable.

In some embodiments, the pharmaceutical compositions and dosage forms comprise anhydrous pharmaceutical compositions and dosage forms. Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Suitable vehicles are well known to those skilled in the art of pharmacy, and non-limiting examples of suitable vehicles include glucose, sucrose, starch, lactose, gelatin, rice, silica gel, glycerol, talc, sodium chloride, dried skim milk, propylene glycol, water, sodium stearate, ethanol, and similar substances well known in the art. Saline solutions and aqueous dextrose and glycerol solutions can also be employed as liquid vehicles. Whether a particular vehicle is suitable for incorporation into a pharmaceutical composition or dosage form depends on a variety of factors well known in the art including, but not limited to, the way in which the dosage form will be administered to a patient and the specific active ingredients in the dosage form. Pharmaceutical vehicles can be sterile liquids, such as water and oils, including those of petroleum, animal, vegetable or synthetic origin, such as peanut oil, soybean oil, mineral oil, sesame oil and the like.

The invention also provides that a pharmaceutical composition can be packaged in a hermetically sealed container such as an ampoule or sachette indicating the quantity. In one embodiment, the pharmaceutical composition can be supplied as a dry sterilized lyophilized powder in a delivery device suitable for administration to the lower airways of a patient. The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for administration may be in the form of powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention (e.g., antibodies) as an active ingredient.

A liquid composition herein can be used as such with a delivery device, or they can be used for the preparation of pharmaceutically acceptable formulations comprising antibodies that are prepared for example by the method of spray drying. The methods of spray freeze-drying proteins for pharmaceutical administration disclosed in Maa et al., Curr. Pharm. Biotechnol., 2001, 1, 283-302, are incorporated herein. In another embodiment, the liquid solutions herein are freeze spray dried and the spray-dried product is collected as a dispersible peptide-containing powder that is therapeutically effective when administered to an individual.

The compounds and pharmaceutical compositions of the present invention can be employed in combination therapies, that is, the compounds and pharmaceutical compositions can be administered concurrently with, prior to, or subsequent to, one or more other desired therapeutics or medical procedures (e.g., antibodies can be used in combination treatment with another treatment such as antivirals or with a vaccine, and/or another treatment). The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another therapeutic or prophylactic).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The current invention provides for dosage forms comprising peptides suitable for treating cancer or other diseases. The dosage forms can be formulated, e.g., as sprays, aerosols, nanoparticles, liposomes, or other forms known to one of skill in the art. See, e.g., Remington's Pharmaceutical Sciences; Remington: The Science and Practice of Pharmacy supra; Pharmaceutical Dosage Forms and Drug Delivery Systems by Howard C., Ansel et al., Lippincott Williams & Wilkins; 7th edition (Oct. 1, 1999).

Generally, a dosage form used in the acute treatment of a disease may contain larger amounts of one or more of the active ingredients it comprises than a dosage form used in the chronic treatment of the same disease. In addition, the prophylactically and therapeutically effective dosage form may vary among different conditions. For example, a therapeutically effective dosage form may contain one or more antibodies that have an appropriate therapeutic action when intending to treat cancer or an autoimmune disease such as Systemic lupus erythematosus (Lupus). On the other hand, a different effective dosage may contain one or more antibodies that have an appropriate prophylactic action when intending to prevent cancer or an autoimmune disease (e.g., Lupus). These and other ways in which specific dosage forms encompassed by this invention will vary from one another and will be readily apparent to those skilled in the art. See, e.g., Remington's Pharmaceutical Sciences, 2005, Mack Publishing Co.; Remington: The Science and Practice of Pharmacy by Gennaro, Lippincott Williams & Wilkins; 20th edition (2003); Pharmaceutical Dosage Forms and Drug Delivery Systems by Howard C. Ansel et al., Lippincott Williams & Wilkins; 7th edition (Oct. 1, 1999); and Encyclopedia of Pharmaceutical Technology, edited by Swarbrick, J. & J. C. Boylan, Marcel Dekker, Inc., New York, 1988, which are incorporated herein by reference in their entirety.

The pH of a pharmaceutical composition or dosage form may also be adjusted to improve delivery and/or stability of one or more active ingredients. Similarly, the polarity of a solvent carrier, its ionic strength, or tonicity can be adjusted to improve delivery. Compounds such as stearates can also be added to pharmaceutical compositions or dosage forms to alter advantageously the hydrophilicity or lipophilicity of one or more active ingredients to improve delivery. In this regard, stearates can also serve as a lipid vehicle for the formulation, as an emulsifying agent or surfactant, and as a delivery enhancing or penetration-enhancing agent. Different salts, hydrates, or solvates of the active ingredients can be used to adjust further the properties of the resulting composition.

Compositions can be formulated with appropriate carriers and adjuvants using techniques to yield compositions suitable for prophylaxis or treatment. The compositions can include an adjuvant, such as, for example but not limited to, alum, poly IC, MF-59, squalene-based adjuvants, or liposomal based adjuvants suitable for prophylaxis or treatment.

In some embodiments, the antibodies described herein are encoded by nucleic acids which are prepared in a mRNA-LNP or a DNA-LNP formulation for administration to a subject.

### Antibody Production

The antibodies (e.g., bispecific antibodies) disclosed herein can be produced by any method known in the art. In some embodiments, the antibodies disclosed herein are produced by culturing a cell transfected or transformed with a vector comprising nucleic acid sequences encoding an antibody described herein and isolating the antibody. E.g., See Example 1. For example, the bispecific antibodies can be produced using chemical cross-linking of two IgG molecules, via fusion of two hybridomas, or via recombinant methods, e.g., via “knobs-into-holes” heterodimerization technology. See Marvin, Jonathan S., and Zhenping Zhu, Recombinant Approaches to IgG-like Bispecific Antibodies, Acta Pharmacologica Sinica 26.6 (2005): 649-658, incorporated by reference in its entirety herein.

In some embodiments, antibodies are synthesized by the hybridoma culture method which results in antibodies that are not contaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques known in the art, including, for example, the hybridoma method (e.g., Kohler and Milstein., Nature, 256:495-97 (1975); Hongo et al, Hybridoma, 14 (3): 253-260 (1995), Harlow et al, Antibodies: A Laboratory Manual, (Cold Spring Harbor Laboratory Press, 2nd ed. 1988); Hammerling et al, in: Monoclonal Antibodies and T-Cell Hybridomas 563-681 (Elsevier, N. Y., 1981)), recombinant DNA methods, phage-display technologies (see, e.g., Clackson et al, Nature, 352: 624-628 (1991); Marks et al, J. MoI Biol. 222: 581-597 (1992); Sidhu et al, J. MoI Biol. 338(2): 299-310 (2004); Lee et al, J. MoI Biol. 340(5): 1073-1093 (2004); Fellouse, Proc. Natl. Acad. ScL USA 101(34): 12467-12472 (2004); and Lee et al, J. Immunol. Methods 284(1-2): 119-132 (2004), and technologies for producing human or humanlike antibodies in animals that have parts or all of the human immunoglobulin loci or genes encoding human immunoglobulin sequences (see, e.g., Lonberg et al, Nature 368: 856-859 (1994); Morrison, Nature 368: 812-813 (1994); Fishwild et al, Nature Biotechnol 14: 845-851 (1996); Neuberger, Nature Biotechnol. 14: 826 (1996); and Lonberg and Huszar, Intern. Rev. Immunol. 13: 65-93 (1995).

In some embodiments, expression of an antibody comprises expression vector(s) containing a polynucleotide that encodes an antiCD3×SLAMF6, an anti-CD45×SLAMF6, or an anti-CD43×SLAMF6 bispecific antibody. Methods that are well known to those skilled in the art can be used to construct expression vectors comprising antibody coding sequences and appropriate transcriptional and translational control signals. These methods include, for example, in vitro recombinant DNA techniques, synthetic techniques, and in vivo genetic recombination. Particular embodiments provide replicable vectors comprising a nucleotide sequence encoding an anti-CD3×SLAMF6, an anti-CD45×SLAMF6, or an anti-CD43×SLAMF6 bispecific antibody disclosed herein operably linked to a promoter. In preferred embodiments, such vectors may include a nucleotide sequence encoding the heavy chain of an antibody molecule (or fragment thereof), a nucleotide sequence encoding the light chain of an antibody (or fragment thereof), or both the heavy and light chain.

In some embodiments, a bispecific antibody described herein is made using the “knob-into-hole” or “KnH” technology. This method involves directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, CL:CH1 interfaces or VH/VL interfaces of antibodies. See, e.g., US2007/0178552; WO 96/027011; WO 98/050431; Zhu et al. (1997) Protein Science 6:781-788, each of which are incorporated in its entirety herein. For example, the production of bispecific antibodies with the KnHs strategy can be based on a single amino acid substitution in the opposite CH3 domains that promotes heavy chain heterodimerization. In some embodiments, in one of the heavy chains referred as a “knob” variant, a small amino acid has been replaced with a larger one in the CH3 domain. Subsequently, in the other heavy chain, a large amino acid has been replaced with a smaller one. A “hole” is formed, permitting the interaction with the “knobs.” See Ridgway J B B, Presta L G, Carter P., “Knobs-into-Holes” Engineering of Antibody CH3 Domains for Heavy Chain Heterodimerization. Protein Eng. 1996, 9:617-621, incorporated in its entirety herein. This method can be used to pair two different heavy chains together during the manufacture of multispecific antibodies such as bispecific antibodies. For example, multispecific antibodies having KnH in their Fc regions can further comprise single variable domains linked to each Fc region, or further comprise different heavy chain variable domains that pair with similar or different light chain variable domains. KnH technology can also be used to pair two different receptor extracellular domains together or any other polypeptide sequences that comprises different target recognition sequences (e.g., including affibodies, peptibodies and other Fc fusions). In some embodiments, the bispecific antibodies described herein are scFv-Fc antibodies. In some embodiments, the scFv-Fc is a bispecific, bivalent molecule which is developed by fusing scFvs with different specificity to each Fc chain. In an scFv-Fc, the knobs-into-holes mutations in Fc force the formation of heterodimer.

The polynucleotide encoding the antibody may be modified, for example, by substituting the coding sequence for human heavy- and light-chain constant domains in place of the homologous murine sequences (U.S. Pat. No. 4,816,567; Morrison, et al, Proc. Natl Acad. ScL USA, 81:6851 (1984)), or by covalently joining to the immunoglobulin coding sequence all or part of the coding sequence for a non-immunoglobulin polypeptide. Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen. The monoclonal antibodies described herein may by monovalent, the preparation of which is well known in the art. For example, one method involves recombinant expression of immunoglobulin light chain and a modified heavy chain. The heavy chain is truncated generally at any point in the Fc region so as to prevent heavy chain crosslinking. Alternatively, the relevant cysteine residues may be substituted with another amino acid residue or are deleted so as to prevent crosslinking. In vitro methods are also suitable for preparing monovalent antibodies. Digestion of antibodies to produce fragments thereof, particularly Fab fragments, can be accomplished using routine techniques known in the art. Chimeric or hybrid antibodies also may be prepared in vitro using known methods in synthetic protein chemistry, including those involving crosslinking agents.

Various expression systems for producing antibodies are known in the art, and include, prokaryotic (e.g., bacteria), plant, insect, yeast, and mammalian expression systems. Suitable cell lines, can be transformed, transduced, or transfected with nucleic acids containing coding sequences for antibodies or portions of antibodies disclosed herein in order to produce the antibody of interest. Expression vectors containing such nucleic acid sequences, which can be linked to at least one regulatory sequence in a manner that allows expression of the nucleotide sequence in a host cell, can be introduced via methods known in the art. Practitioners in the art understand that designing an expression vector can depend on factors, such as the choice of host cell to be transfected and/or the type and/or amount of desired protein to be expressed. Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication. For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest (e.g., antibodies and fragments thereof) is stably integrated into the genome of eukaryotic cells (for example mammalian cells), resulting in the stable expression of transfected genes. A gene that encodes a selectable marker (for example, resistance to antibiotics or drugs) can be introduced into host cells along with the gene of interest in order to identify and select clones that stably express a gene encoding a protein of interest. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired antibody molecule.

In some embodiments, the bispecific antibodies disclosed herein are encoded in a vector for expression in a cell line. In some embodiments, a first vector comprises a polynucleotide sequence that encodes an anti-CD3 portion of a bispecific antibody, a second vector comprises a polynucleotide sequence that encodes an anti-SLAMF6 portion of a bispecific antibody, and each vector is transfected into one or more cell lines for expression. In some embodiments, a single vector comprises a polynucleotide sequence that encodes an anti-CD3 portion of a bispecific antibody and an anti-SLAMF6 portion of the bispecific antibody and the vector is transfected into one or more cell lines for expression. In some embodiments, one or more vectors comprise polynucleotide sequences encoding a light chain or a fragment thereof and a heavy chain or a fragment thereof the bispecific antibody. For example, in some embodiments, a first vector may comprise a polynucleotide sequence encoding a light chain, or fragment thereof, of an anti-CD3 portion of the bispecific antibody, a second vector may comprise a polynucleotide sequence encoding a light chain, or fragment thereof, of an anti-SLAMF6 portion of the bispecific antibody, a third vector may comprise a polynucleotide sequence encoding a heavy chain, or fragment thereof, of an anti-CD3 portion of the bispecific antibody, and/or a fourth vector may comprise a polynucleotide sequence encoding a heavy chain, or fragment thereof, of an anti-SLAMF6 portion of the bispecific antibody. In some embodiments, all four vectors are transfected into one or more cell lines for expression.

In some embodiments, the bispecific antibodies disclosed herein are encoded in a vector for expression in a cell line. In some embodiments, a first vector comprises a polynucleotide sequence that encodes an anti-CD45 portion of a bispecific antibody, a second vector comprises a polynucleotide sequence that encodes an anti-SLAMF6 portion of a bispecific antibody, and each vector is transfected into one or more cell lines for expression. In some embodiments, a single vector comprises a polynucleotide sequence that encodes an anti-CD45 portion of a bispecific antibody and an anti-SLAMF6 portion of the bispecific antibody and the vector is transfected into one or more cell lines for expression. In some embodiments, one or more vectors comprise polynucleotide sequences encoding a light chain or a fragment thereof and a heavy chain or a fragment thereof the bispecific antibody. For example, in some embodiments, a first vector may comprise a polynucleotide sequence encoding a light chain, or fragment thereof, of an anti-CD45 portion of the bispecific antibody, a second vector may comprise a polynucleotide sequence encoding a light chain, or fragment thereof, of an anti-SLAMF6 portion of the bispecific antibody, a third vector may comprise a polynucleotide sequence encoding a heavy chain, or fragment thereof, of an anti-CD45 portion of the bispecific antibody, and/or a fourth vector may comprise a polynucleotide sequence encoding a heavy chain, or fragment thereof, of an anti-SLAMF6 portion of the bispecific antibody. In some embodiments, all four vectors are transfected into one or more cell lines for expression.

In some embodiments, the bispecific antibodies disclosed herein are encoded in a vector for expression in a cell line. In some embodiments, a first vector comprises a polynucleotide sequence that encodes an anti-CD43 portion of a bispecific antibody, a second vector comprises a polynucleotide sequence that encodes an anti-SLAMF6 portion of a bispecific antibody, and each vector is transfected into one or more cell lines for expression. In some embodiments, a single vector comprises a polynucleotide sequence that encodes an anti-CD43 portion of a bispecific antibody and an anti-SLAMF6 portion of the bispecific antibody and the vector is transfected into one or more cell lines for expression. In some embodiments, one or more vectors comprise polynucleotide sequences encoding a light chain or a fragment thereof and a heavy chain or a fragment thereof the bispecific antibody. For example, in some embodiments, a first vector may comprise a polynucleotide sequence encoding a light chain, or fragment thereof, of an anti-CD43 portion of the bispecific antibody, a second vector may comprise a polynucleotide sequence encoding a light chain, or fragment thereof, of an anti-SLAMF6 portion of the bispecific antibody, a third vector may comprise a polynucleotide sequence encoding a heavy chain, or fragment thereof, of an anti-CD43 portion of the bispecific antibody, and/or a fourth vector may comprise a polynucleotide sequence encoding a heavy chain, or fragment thereof, of an anti-SLAMF6 portion of the bispecific antibody. In some embodiments, all four vectors are transfected into one or more cell lines for expression.

A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of protein products may be important for the function of the antibody. Different host cell strains have characteristic and specific mechanisms for the post-translational processing and modification of proteins and gene products. As such, appropriate host systems or cell lines can be chosen to ensure the correct modification and processing of the foreign antibody expressed. Thus, eukaryotic host cells possessing the cellular machinery for proper processing of the primary transcript, glycosylation, and phosphorylation of the gene product may be used.

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available media can be utilized.

Bispecific antibodies disclosed herein can be purified from any human or non-human cell which expresses the antibody, including those which have been transfected with expression constructs that express the antibody or fragments thereof. For antibody recovery, isolation and/or purification, the cell culture medium or cell lysate is centrifuged to remove particulate cells and cell debris. The desired antibody molecule is isolated or purified away from contaminating soluble proteins and polypeptides by suitable purification techniques. Non-limiting purification methods for proteins/antibodies include: size exclusion chromatography; affinity chromatography; ion exchange chromatography; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

### Methods of Treatment

In one embodiment, the subject matter disclosed herein relates to a preventive medical treatment started after following diagnosis of a disease (e.g., cancer, Lupus) in order to prevent the disease from worsening or curing the disease. In one embodiment, the subject matter disclosed herein relates to prophylaxis of subjects who are believed to be at risk for moderate or severe disease associated with cancer or have previously been diagnosed with another disease, such as cancer. In one embodiment, the subjects can be administered the pharmaceutical composition described herein. The invention contemplates using any of the antibodies produced by the systems and methods described herein. In one embodiment, the compositions described herein can be administered subcutaneously via syringe or any other suitable method know in the art.

The compound(s) or combination of compounds disclosed herein, or pharmaceutical compositions may be administered to a cell, mammal, or human by any suitable means. Non-limiting examples of methods of administration include, among others, (a) administration though oral pathways, which includes administration in capsule, tablet, granule, spray, syrup, or other such forms; (b) administration through non-oral pathways such as intraocular, intranasal, intraauricular, rectal, vaginal, intraurethral, transmucosal, buccal, or transdermal, which includes administration as an aqueous suspension, an oily preparation or the like or as a drip, spray, suppository, salve, ointment or the like; (c) administration via injection, including subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; (e) administration topically; as deemed appropriate by those of skill in the art for bringing the compound or combination of compounds disclosed herein into contact with living tissue; (f) administration via inhalation, including through aerosolized, nebulized, and powdered formulations; (g) administration through implantation; and administration via electroporation.

In some embodiments, one or more antibodies disclosed herein are prepared in a cocktail of DNA-encoding antibodies or mRNA-encoding antibodies and delivered by electroporation to a subject for in vivo expression of the encoded antibodies.

As will be readily apparent to one skilled in the art, the effective in vivo dose to be administered and the particular mode of administration will vary depending upon the age, weight and species treated, and the specific use for which the compound or combination of compounds disclosed herein are employed. The determination of effective dose levels, that is the dose levels necessary to achieve the desired result, can be accomplished by one skilled in the art using routine pharmacological methods. Typically, human clinical applications of products are commenced at lower dose levels, with dose level being increased until the desired effect is achieved. Alternatively, acceptable in vitro studies can be used to establish useful doses and routes of administration of the compositions identified by the present methods using established pharmacological methods. Effective animal doses from in vivo studies can be converted to appropriate human doses using conversion methods known in the art (e.g., see Nair A B, Jacob S. A simple practice guide for dose conversion between animals and human. Journal of basic and clinical pharmacy. 2016 March; 7(2):27.)

### Methods of Prevention

In some embodiments, the compositions prepared using methods of the invention can be used as a vaccine to promote an immune response against future disease (e.g., cancer) or an autoimmune disease (e.g., Lupus). In some embodiments, the antibodies are neutralizing antibodies.

In some embodiments, the antibodies (or polynucleotides encoding antibodies) prepared using methods of the invention can be combined with additional pharmaceutical components.

### Dosage

A prophylactically effective or therapeutically effective amount is typically dependent on the weight of the subject being treated, the subject's physical condition, the extensiveness of the condition to be treated, and the age of the subject being treated. In general, an anti-CD3×SLAMF6, an anti-CD45×1SLAMF6, or an anti-CD43×SLAMF6 bispecific antibody, or polynucleotides encoding one or more antibodies, disclosed herein may be administered in an amount in the range of about 10 ng/kg body weight to about 100 mg/kg body weight per dose. In some embodiments, antibodies may be administered in an amount in the range of about 50 μg/kg body weight to about 5 mg/kg body weight per dose. In some embodiments, antibodies may be administered in an amount in the range of about 100 μg/kg body weight to about 10 mg/kg body weight per dose. In some embodiments, antibodies may be administered in an amount in the range of about 100 μg/kg body weight to about 20 mg/kg body weight per dose. In some embodiments, antibodies may be administered in an amount in the range of about 0.5 mg/kg body weight to about 20 mg/kg body weight per dose. In some embodiments, antibodies may be administered in an amount in the range of about 0.5 mg/kg body weight to about 10 mg/kg body weight per dose. In some embodiments, antibodies may be administered in an amount in the range of about 1 mg/kg body weight to about 5 mg/kg body weight per dose. In some embodiments, antibodies may be administered in an amount in the range of about 0.1 mg/kg body weight to about 0.5 mg/kg body weight per dose. In some embodiments, antibodies may be administered in a dose of at least about 100 μg/kg body weight, at least about 250 μg/kg body weight, at least about 500 μg/kg body weight, at least about 750 μg/kg body weight, at least about 3 mg/kg body weight, at least about 5 mg/kg body weight, or at least about 10 mg/kg body weight.

In some methods, the dosage is adjusted to achieve a plasma antibody concentration of about 1-1000 μg/mL or about 25-300 μg/mL. In some embodiments, the dosage is adjusted to achieve a plasma antibody concentration of about 0.001 μg/mL to about 10 μg/mL. In some embodiments, the dosage is adjusted to achieve a plasma antibody concentration of about 1 μg/mL to about 10 μg/mL. In some embodiments, the dosage is adjusted to achieve a plasma antibody concentration of about 0.01 μg/mL to about 1 μg/mL. In some embodiments, the dosage is adjusted to achieve a plasma antibody concentration of about 0.01 μg/mL to about 0.1 μg/mL.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—SLAMF6 Compartmentalization Enhances T Cell Functions

### SLAMF6 Mediated T Cell Activation Requires Spatial Co-Localization of SLAMF6 and CD3 Along the Cell Membrane

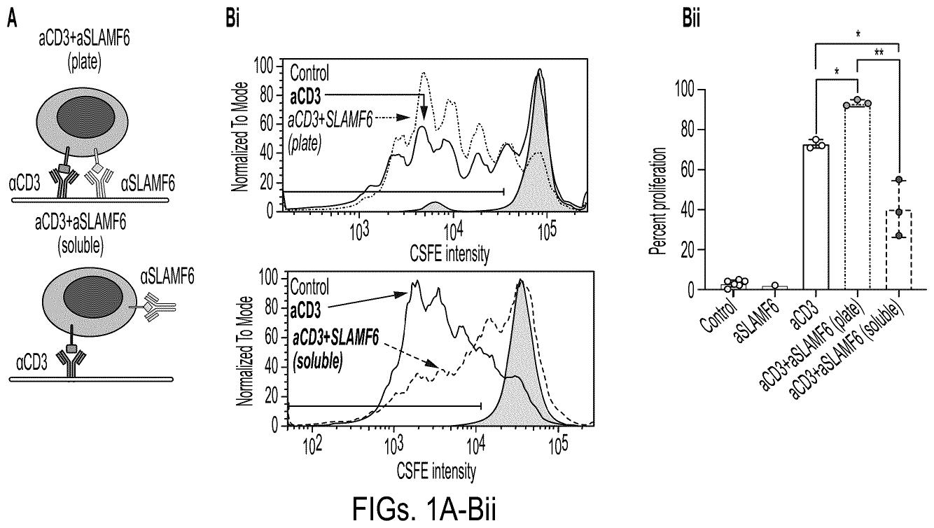

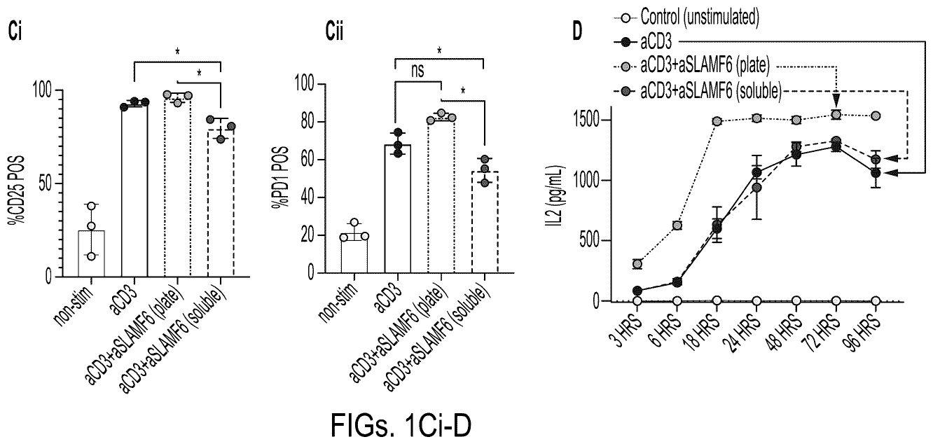

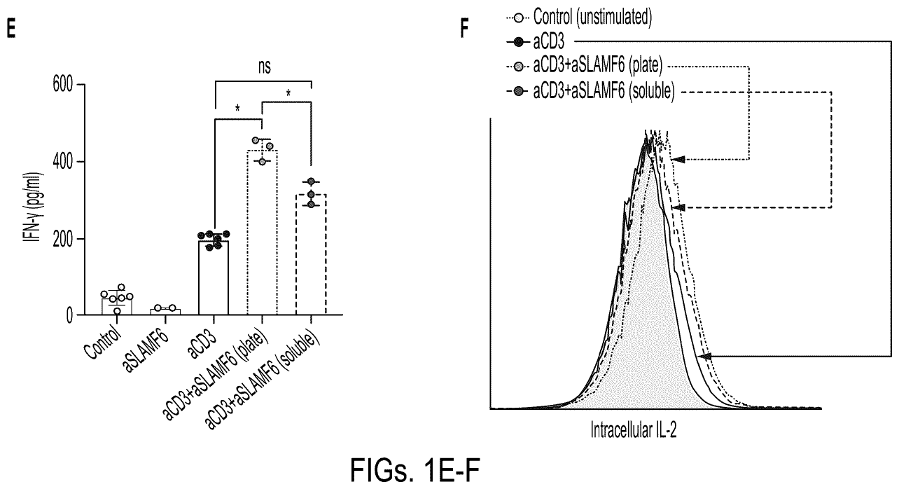

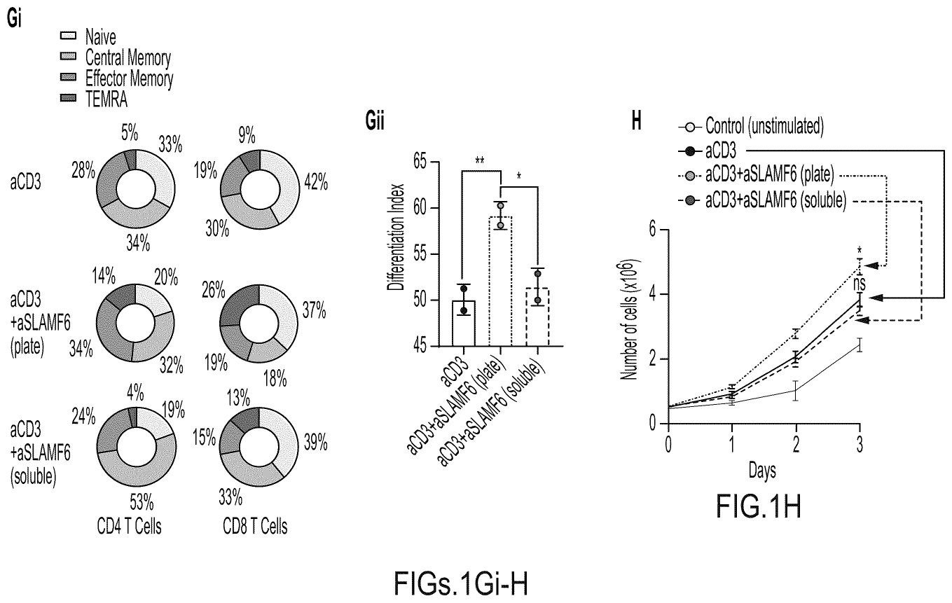

In previous work we had shown that SLAMF6 engagement, in the presence of CD3 ligation, results in increased T cell activity as measured by cytokine secretion (interleukin 2 (IL-2) and IFN-γ), cell proliferation, and phosphorylation of proximal TCR signaling proteins. Dragovich, M. A., et al., SLAMF6 clustering is required to augment T cell activation. PLoS One, 2019. 14(6): p. e0218109. This activation is dependent on colocalization of SLAMF6 with the CD3 in the IS. To evaluate whether SLAMF6 signaling can be modulated by changing the spatial location of the receptor with respect to the TCR, we set up two stimulation conditions to mimic SLAMF6 receptor clustering versus separation from the CD3. In the first condition, T cells were stimulated with immobilized anti-CD3 and anti-SLAMF6 antibodies on a plate surface, thereby allowing the two receptors to cluster together. In the second condition T cells were stimulated with immobilized anti-CD3 but soluble anti-SLAMF6 antibodies, introducing spatial separation between the activated SLAMF6 and CD3 receptors on the cell surface (FIG. 1A). Equal loading of anti-SLAMF6 was ensured across all conditions.

Ligation of the SLAMF6 and CD3 receptors in the plate bound SLAMF6 (plate) condition resulted in an increase in T cell proliferation as compared to ligation of CD3 alone (soluble) (FIG. 1Bi). However, stimulation in the soluble SLAMF6 condition resulted in inhibition of T cell proliferation that occurred as a result of spatial separation of CD3 and SLAMF6 at time of receptor engagement by anti-CD3 and anti-SLAMF6 antibodies respectively (FIG. 1Bi and as quantified in FIG. 1Bii). Cell surface expression of the activation markers CD25 (FIG. 1Ci) and PD-1 (FIG. 1Cii), as well as the levels of secreted IL-2 (FIG. 1D) and IFN-γ (FIG. 1E) were decreased in the soluble SLAMF6 as compared to the plate bound SLAMF6 condition, mirroring the decreased activation that was seen in the proliferation assay (FIG. 1A). Similarly, following a brief four-hour stimulation, there was a mild increase intracellular IL-2 levels in the plate bound SLAMF6 as compared to the soluble SLAMF6 condition (FIG. 1F). We also evaluated T cell differentiation following a five-day stimulation. Cells states were defined as naïve (N; CD45RA⁺CCR7⁺), central memory (CM; CD45RA⁻CCR7⁺), effector memory (EM; CD45RA⁻CCR7⁻), and terminally differentiated effector memory (TEMRA; CD45RA⁺CCR7⁻). In the plate bound SLAMF6 condition, as compared to the soluble SLAMF6 condition, percentages of N cells were similar, CM were reduced, whereas those of EM and TEMRA cells were increased (FIG. 1Gi). The mean T cell maturation index ([1×N+2×CM+3×EM+4×TEMRA]/4) for cells stimulated with anti-CD3 antibody alone, anti-CD3+anti-SLAMF6 (plate), and anti-CD3+anti-SLAMF6 (soluble) was 50.1, 59.4, and 51.4, respectively (one-way ANOVA p=0.001) (FIG. 1Gii). Finally, we quantified the cell number of Jurkat T cells stimulated in culture over three days. The absolute cell count was significantly greater in the plate bound SLAMF6 condition as compared to the soluble SLAMF6 stimulation condition (FIG. 1H). Thus, ligation of SLAMF6 in proximity to CD3 results in net activation of T cell signaling, as measured by cell proliferation, upregulation of cell surface activation markers, cytokine release and T cell maturation. Spatially removing the SLAMF6 receptor from the CD3 receptor dampens this activation signal and constrains T cell proliferation. Hence, the location of SLAMF6 in reference to CD3 modulates the net effect of SLAMF6 signaling on T cell activity, with the potential to alter the signal from activation to inhibition.

SLAMF6 Co-Immunoprecipitation (Co-IP) Assay Identifies Downstream Proteins that Bridge SLAMF6 and TCR Signaling.

Having observed that SLAMF6 has the ability to both excite and dampen T cell functions, modified by its location with respect to the CD3, we hypothesized that signal regulation is dependent on the availability of downstream proteins that bridge the SLAMF6 and CD3 signals. Specifically, activation following ligation of SLAMF6 and/or CD3 is dependent on SRC tyrosine kinases (LCK and FYN) that are brought into close proximity with their substrates when SLAMF6 and CD3 cluster together in the IS. To evaluate whether forced removal of SLAMF6 from CD3 results in a “steal” of signaling molecules away from the TCR, we performed a SLAMF6 co-IP assay with SLAMF6 clustered with CD3 (plate bound SLAMF6) as compared with SLAMF6 that is spatially removed from CD3 (soluble SLAMF6). Jurkat T cells expressing V5 tagged SLAMF6 were stimulated with anti-CD3 and anti-SLAMF6 antibodies (plate SLAMF6 versus soluble SLAMF6 conditions, as above), after which cells were lysed and immunoprecipitated with anti-V5 antibodies to enrich the SLAMF6 interactome. The SLAMF6 immunoprecipitated reaction was submitted for mass spectrometry analysis to identify SLAMF6 interacting proteins. We hypothesized that key regulatory signaling proteins would remain associated with SLAMF6 in the soluble SLAMF6 condition, suggesting their removal from the CD3 site contributes to the inhibition seen in the soluble as compared to the plate SLAMF6 condition. In the analyzed co-IP samples, the bait, SLAMF6, was present in equal amounts in the plate and soluble SLAMF6 conditions, reflecting the quantitative robustness of both the co-IP conditions in addition to the quality control assessment in terms of equal loading of peptides from each condition.

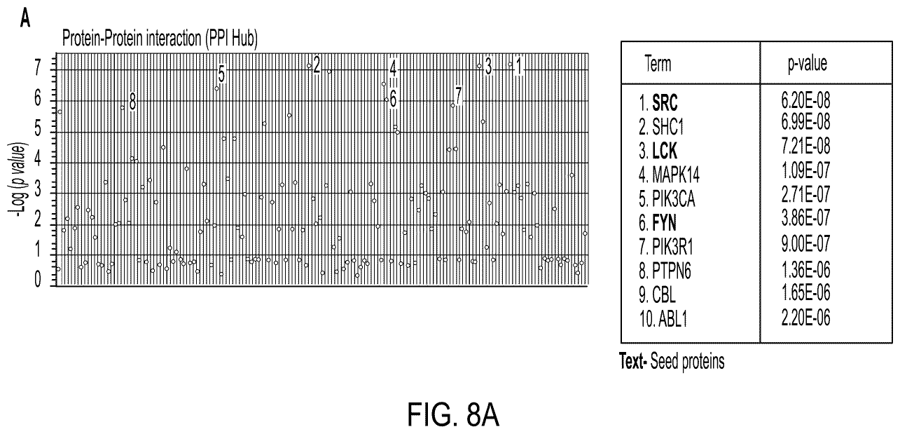

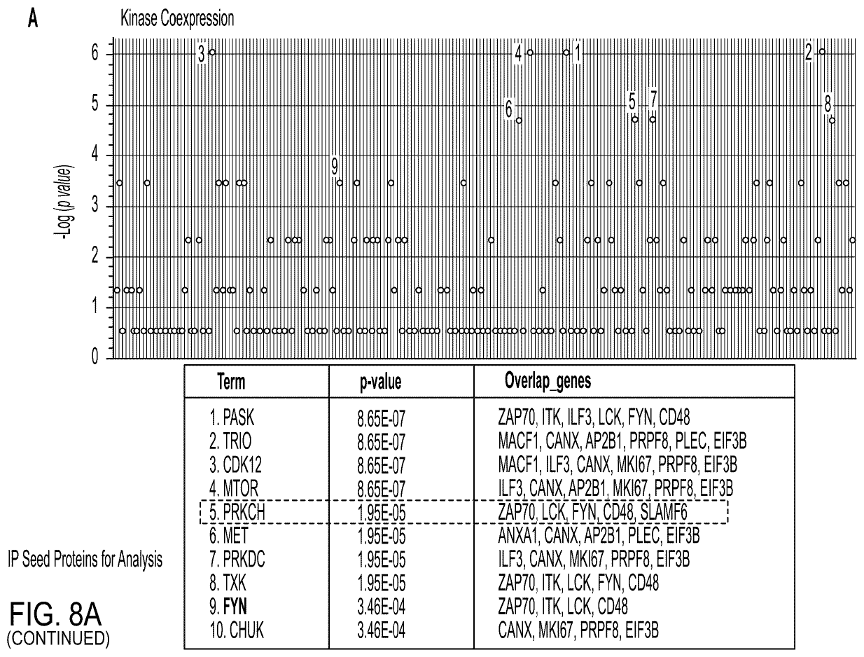

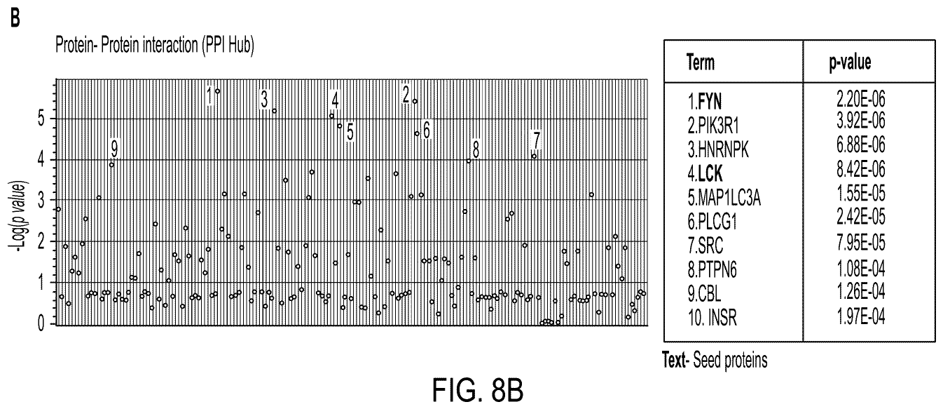

An unbiased protein enrichment pathway analysis (please refer to FIG. 8 for a complete list of seed proteins) identified that the TCR signaling pathway and the primary immunodeficiency pathway, regardless of plate versus soluble SLAMF6 stimulation, are the most likely pathways to interact with SLAMF6 (FIG. 2Ai and FIG. 2Aii). This intimate association of SLAMF6 pulldown proteins with TCR signaling proteins highlight why receptor clustering along the cell surface membrane is so important for downstream signal propagation. Subsequently, we restricted our analysis to proteins involved in the proximal T cell signaling pathway; these included LCK, FYN, and ZAP70 amongst others (FIG. 2B). We found a non-differential pulldown of essential T cell signaling proteins in both the soluble and plate SLAMF6 conditions, suggesting they remain associated with SLAMF6 even when the latter is spatially removed from the CD3 site. Because SLAMF6, when spatially removed from CD3, was still associated with many of the signaling proteins essential for TCR signaling, we consider this “steal” of LCK, FYN and ZAP70 away from the CD3 to be a contributing factor to the inhibition of TCR activity seen in the soluble SLAMF6 condition. A cartoon diagram of the identified SLAMF6 interactome in TCR signaling is shown (FIG. 2C). The tight network of association that exists between the proteins identified in the SLAMF6 interactome and those known to be essential for the proximal TCR signaling cascade is evident. Finally, we performed a protein-protein interaction analysis to identify the downstream kinases that are predicted to function downstream of SLAMF6 activation in both plate versus soluble SLAMF6 stimulation (FIGS. 8A-B). The predicted kinases differ in the two stimulation conditions, suggesting that while both plate and soluble SLAMF6 stimulation signal via the TCR, these two activation conditions are not identical in downstream signaling events.

In summary, we took advantage of two T cell stimulation conditions, activation with plate bound anti-SLAMF6 and partial-inhibition with soluble anti-SLAMF6, to identify the intracellular binding partners associated with the SLAMF6 receptor. IP proteins involved in T cell signaling pathways predominated in both conditions. We found no differential expression of proximal signaling proteins, suggesting that these SLAMF6 associated signaling molecules move along the cell surface together with the receptor. Thus, when SLAMF6 is spatially separated from the CD3 receptor, it has the potential to “steal” the downstream signaling molecules involved in TCR activation, thereby contributing to cell inhibition (FIG. 2C).

SLAMF6 Mediated T Cell Activation is Enhanced when SLAMF6 and CD3 Cluster in the Immunologic Synapse.

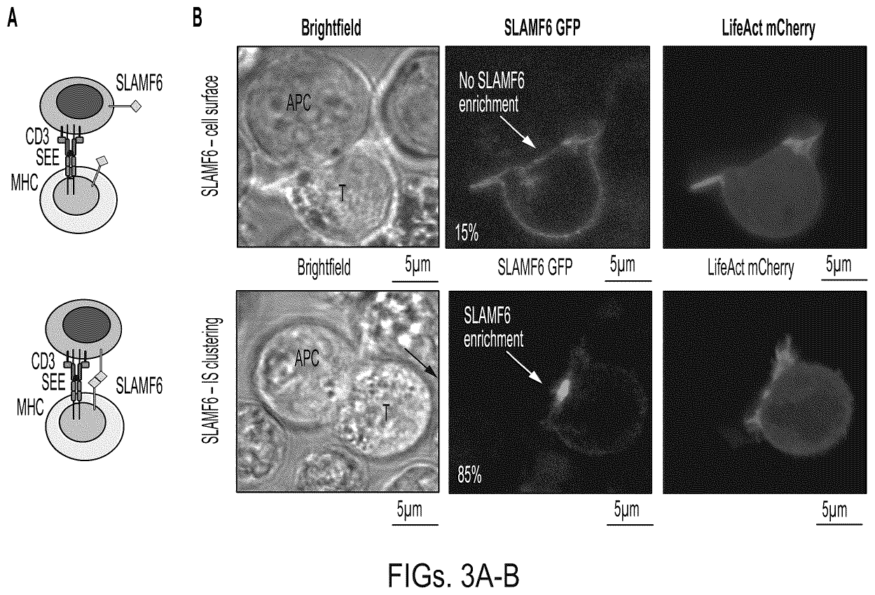

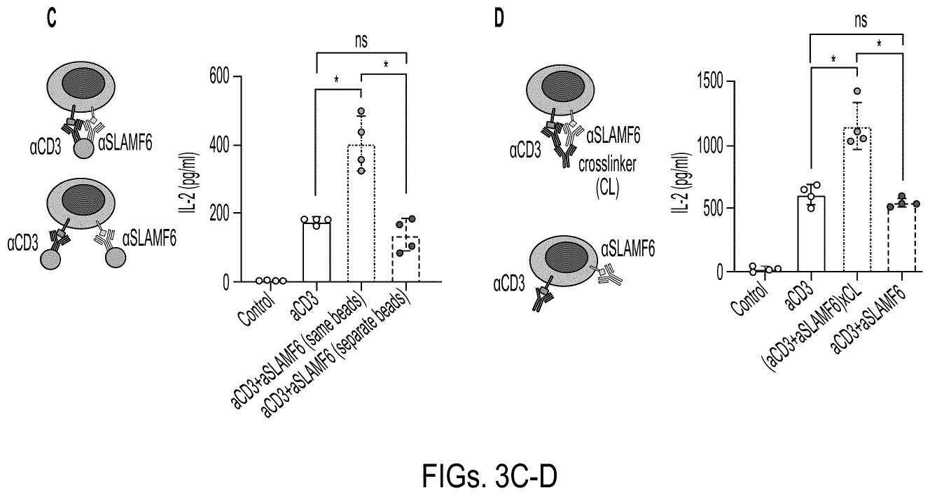

We hypothesized that the function of the SLAMF6 receptor is dependent on its ability to translocate to the IS at time of cell stimulation. First, we wanted to visualize the location of the SLAMF6 receptor in reference to the IS. Jurkat T cells were transfected with LifeAct mCherry (actin label) and GFP-SLAMF6, then co-cultured with Raji B cell loaded with SEE (FIG. 3A). The IS was defined by visualization of actin accumulation at the contact zone. Microscopy imaging revealed that there were two predominant patterns of SLAMF6 expression. Specifically, in some cells SLAMF6 expression was evenly distributed across the cell surface membrane (FIG. 3B top row) whereas SLAMF6 enrichment at the site of the IS could be seen in others (FIG. 3B bottom row). The former occurred in resting T cells, as well as in 15% of the cells that formed synapse with another cell; while the remaining 85% of cells that formed synapses showed SLAMF6 enrichment at the site of the contact. Thus, we visually observed that SLAMF6 is able to translocate and cluster in the IS following cell stimulation.

To test our hypothesis that physical separation versus clustering of the SLAMF6 with the CD3 receptor affects T cell function, we designed beads conjugated with anti-CD3, anti-SLAMF6 or anti-CD3+anti-SLAMF6 antibodies (FIG. 3C). Compared to T cells stimulated with anti-CD3 alone, stimulation with anti-CD3+anti-SLAMF6 conjugated beads resulted in increased IL-2 release whereas stimulation with anti-CD3 beads admixed with anti-SLAMF6 beads failed to augment T cell activity with a trend towards inhibition as compared to stimulation with anti-CD3 alone (FIG. 3C). We repeated the experiment using an Fc crosslinking antibody to cluster the anti-CD3 and anti-SLAMF6 stimulating antibodies on the surface of the T cell. We again saw increased IL-2 secretion as compared to stimulation with anti-CD3 alone or anti-CD3 and anti-SLAMF6 randomly admixed in solution (FIG. 3D), suggesting that colocalization of the SLAMF6 with the CD3 augments downstream T cell activation.

We were thus able to show that SLAMF6 receptors can be found either dispersed from or clustered with the CD3 receptor in the IS. Physically promoting SLAMF6 clustering with CD3 at time of stimulation, using either a system of conjugated beads or cross-linkers, resulted in enhanced T cell activation and established SLAMF6 as an activating co-receptor when localized in proximity to CD3. On the other hand, T cell activation was dampened when SLAMF6 was spatially removed from the CD3 complex, suggesting loss of synergism between these signaling pathways.

T Cell Stimulation with Anti-CD3 SLAMF6 Bispecific Antibody Enhances T Cell Activity

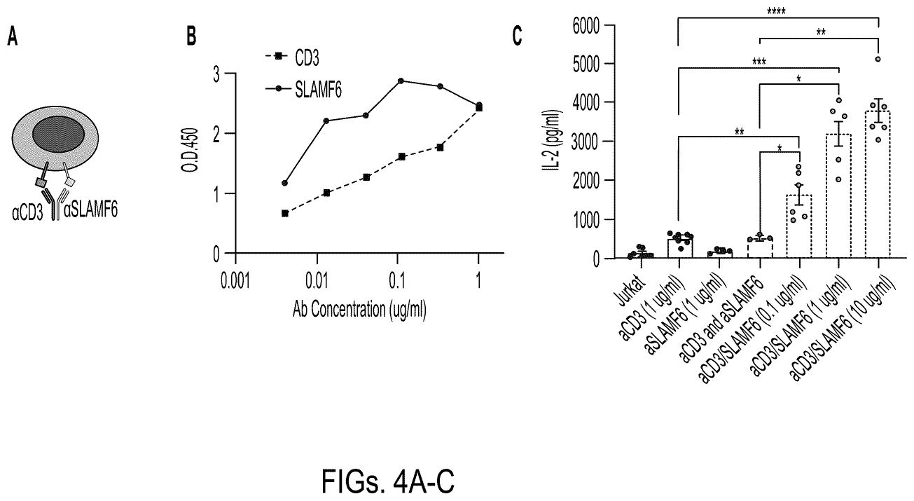

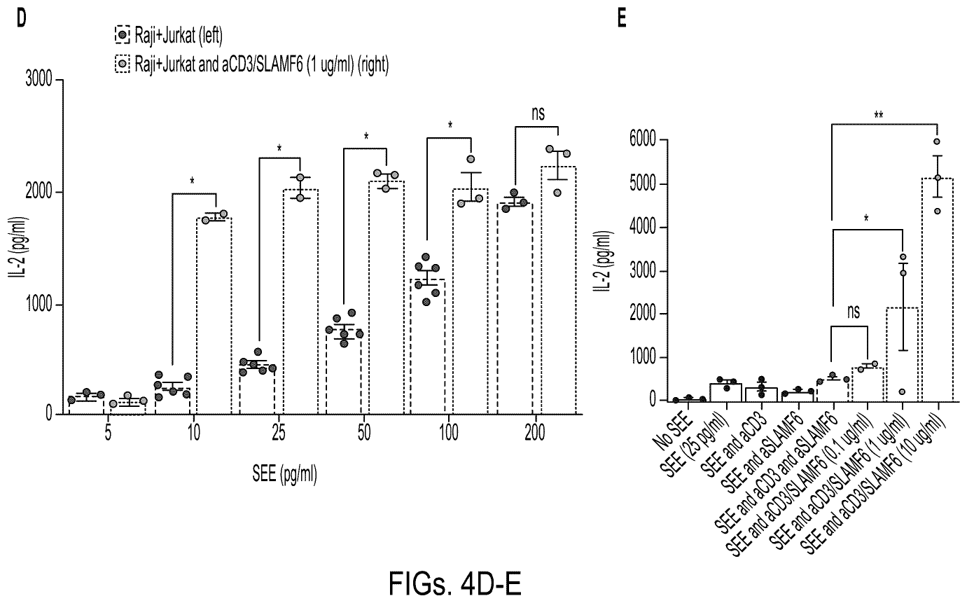

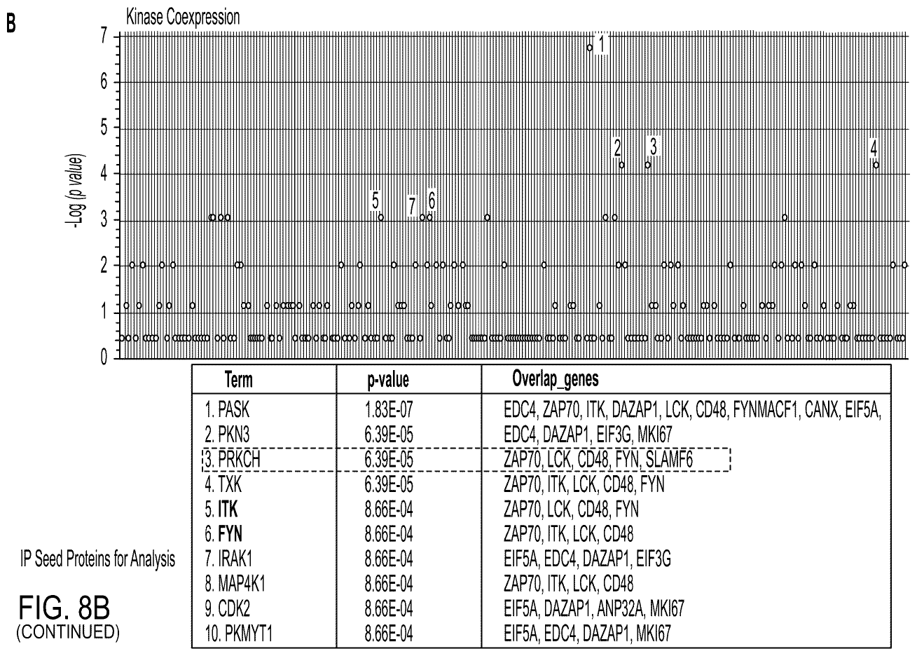

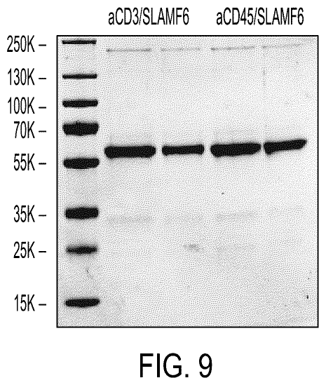

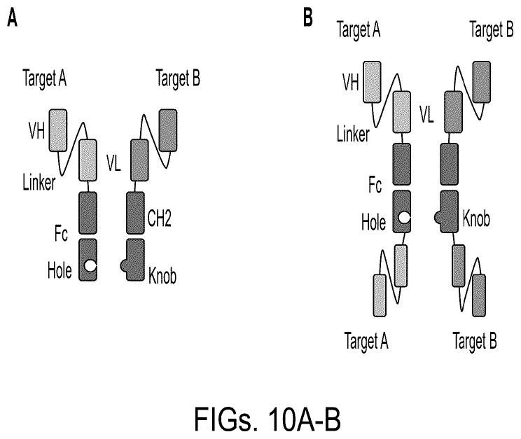

If the signal from a co-receptor can be further adjusted by changing its location along the cell surface membrane, we wondered whether interventions targeting receptor clustering may have a role in regulating cell signaling. We designed a bispecific monoclonal antibody simultaneously targeting CD3 and SLAMF6 (anti-CD3/SLAMF6) with the a priori hypothesis that it will augment T cell activation by promoting, at time of receptor engagement, the clustering of SLAMF6 in proximity to the CD3 (FIG. 4A). The bispecific antibody was generated by co-expression of monovalent OKT3-IgG-hole (SEQ ID NO: 2) and monovalent anti-SLAMF6-IgG-knob (SEQ ID NO: 9) constructs in 293 cells (FIG. 9 and FIGS. 10A-B). We validated the bispecific antibody binding by means of an ELISA assay in which SLAMF6 expressing RAJI cells lysate and SLAMF6-KO Jurkat T cells lysate were used as immobilized antigens, respectively (FIG. 4B).

To assess the biological activity of the bispecific antibody, Jurkat T cells were stimulated with anti-CD3 alone, anti-SLAMF6 alone, anti-CD3 in mixture with anti-SLAMF6, and with the bispecific anti-CD3/SLAMF6 antibody. As we had predicted, increased IL-2 secretion was seen following stimulation with anti-CD3/SLAMF6 as compared to either anti-CD3 alone or anti-CD3 in combination with soluble anti-SLAMF6 (FIG. 4C). A dose response with the use of the bispecific antibody was seen, with greater T cell activation at higher concentrations of anti-CD3/SLAMF6 antibody used for stimulation.

To validate the functional activity of anti-CD3/SLAMF6 in a more physiological system with an intact IS, we used the bispecific antibody to stimulate a co-culture of Jurkat T cells with Raji B cells in presence of Staphylococcus Enterotoxin E (SEE). We again found that the addition of anti-CD3/SLAMF6 increased IL-2 production, almost irrespective of the amount of SEE added (FIG. 4D). The increase in IL-2 release seen with the bispecific anti-CD3/SLAMF6 antibody, as compared to anti-CD3 alone, was not seen when anti-SLAMF6 antibody was added as a soluble admixture (FIG. 4E). These findings are consistent with the initial observation that it is the clustering of SLAMF6 with CD3, which occurs as a result of the physical bridging by the bispecific antibody, that is required for net T cell activation. Additionally, higher concentrations of the anti-CD3/SLAMF6 resulted in greater IL-2 release, suggesting dose response to the use of this bispecific antibody (FIG. 4E).

We therefore show that the novel design of an anti-CD3/SLAMF6 bispecific monoclonal antibody enhances T cell activation greater than either antibody does individually or in combination. Furthermore, a dose dependent effect exists such that greater T cell activation occurs at higher concentrations of bispecific anti-CD3/SLAMF6 antibody used.

T Cell Stimulation with Anti-CD45 SLAMF6 Bispecific Antibody Works Predominantly in Cis to Augment T Cell Activation.

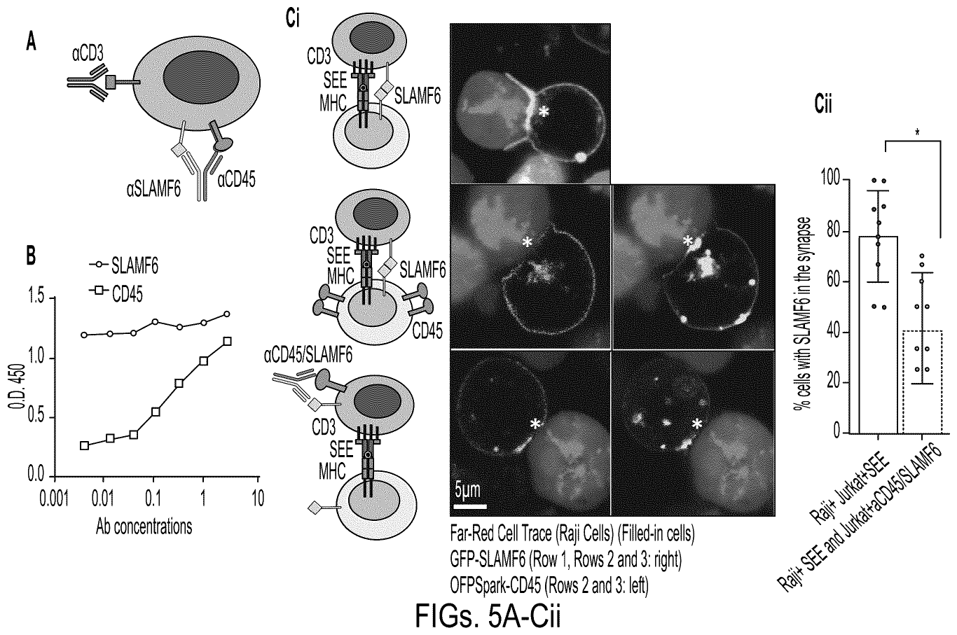

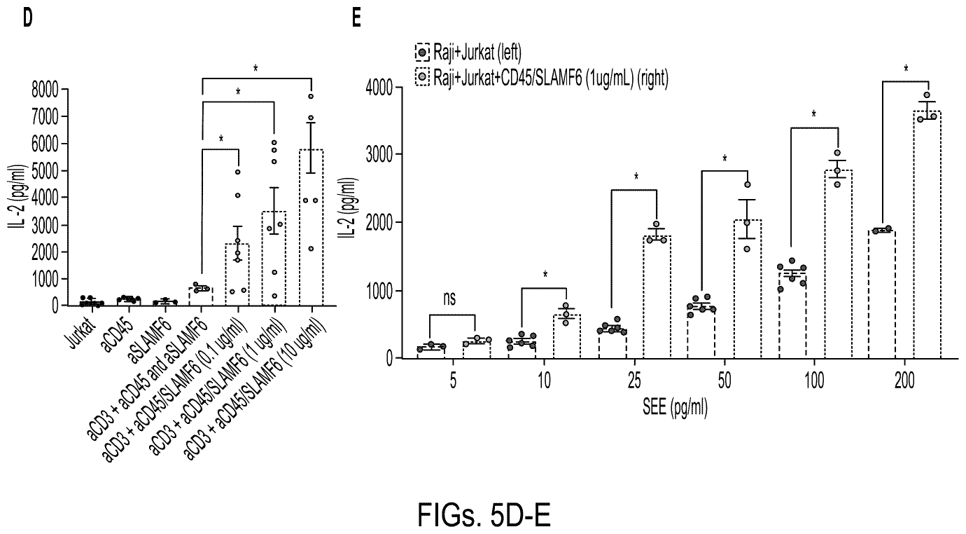

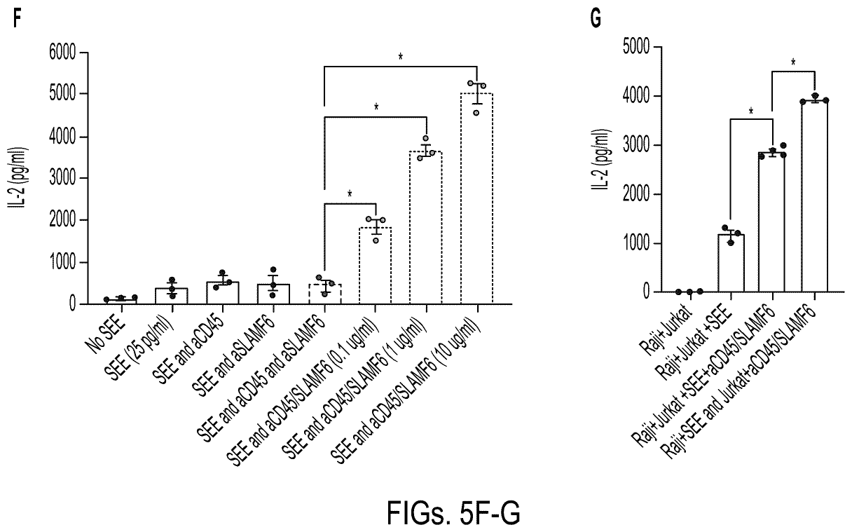

Having shown that a bispecific antibody can augment T cell activation by promoting receptor clustering, we next sought to evaluate whether T cell inhibition can similarly be achieved following forced disruption of receptors' colocalization. Specifically, we hypothesized that SLAMF6-mediated T cell signaling could be inhibited if the SLAMF6 receptor can be tethered away from the IS. We planned to accomplish this using a bispecific anti-CD45/SLAMF6 antibody. CD45 is a receptor protein tyrosine phosphatase expressed on all leukocytes. It is a large glycoprotein of 180-220 kDa with a bulky ectodomain region. While its presence is essential for the initiation of T cell activation, the large CD45 phosphatase is ultimately excluded from the narrow-spaced and matured immunologic synapse as a result of steric hindrance. Choudhuri, K., et al., T-cell receptor triggering is critically dependent on the dimensions of its peptide-MHC ligand. Nature, 2005. 436(7050): p. 578-82; Cordoba, S. P., et al., The large ectodomains of CD45 and CD148 regulate their segregation from and inhibition of ligated T-cell receptor. Blood, 2013. 121(21): p. 4295-302; Chang, V. T., et al., Initiation of T cell signaling by CD45 segregation at ‘close contacts’. Nat Immunol, 2016. 17(5): p. 574-582. To spatially segregate SLAMF6 from the CD3, we sought to create a bispecific monoclonal anti-CD45-SLAMF6 antibody that would bind the SLAMF6 receptor, tethering it to CD45 and thereby excluding it from the immunologic synapse and the CD3 contact zone (FIG. 5A).

We designed the anti-CD45/SLAMF6 bispecific antibody as a fusion immunoglobulin of CD45-IgG-hole (SEQ ID NO: 5) and SLAMF6-IgG-knob (SEQ ID NO: 9) (FIG. 9 and FIGS. 10A-B). Binding to the intended targets was validated using an ELISA assay, with the immobilized ectodomain of CD45 peptide and SLAMF6 peptide used as antigen bait (FIG. 5B). We next sought to evaluate whether treatment with the anti-CD45/SLAMF6 antibody would interfere with SLAMF6 enrichment in the IS. To visualize this, we used confocal microscopy imaging of GFP-tagged SLAMF6 and OFPSpark tagged CD45 in a Jurkat-Raji co-culture. Specifically, Jurkat T cells expressing GFP tagged SLAMF6 were co-cultured with Far Red fluorescently tagged Raji B cells loaded with SEE. Enrichment of SLAMF6 in areas of synapse formation was seen in 79% of the imaged synapses (FIG. 5Ci first row and 5Cii). Next, we transfected T cells to express GFP-SLAMF6 and OFPSpark-CD45. Once again, we saw SLAMF6 enrichment in the IS, but this time we could also visualize CD45 exclusion from the synapse (FIG. 5Ci second row). Finally, we pre-treated the Jurkat T cells with anti-CD45/SLAMF6 antibody prior to the co-culture with Raji B cells (FIG. 5Ci third row). Anti-CD45/SLAMF6 treatment resulted in significant decrease in overall number of synapses, as well as in a reduction of SLAMF6 enrichment in the IS (FIG. 5Cii). On visual inspection, SLAMF6 localization was similar to that of CD45; with both being absent from the IS. These findings suggest that SLAMF6, restrained by the bulky CD45, was indeed excluded from the IS in mature synapses.

We next sought to evaluate the functional effect of anti-CD45/SLAMF6 on T cell activation. Distinct of our original hypothesis, stimulation of Jurkat T cells with anti-CD45/SLAMF6 antibodies in presence of anti-CD3 resulted in enhanced T cell activation as compared to either anti-CD3 alone or in combination with soluble anti-SLAMF6 (FIG. 5D). IL-2 release increased with increasing doses of bispecific antibody (FIG. 5D). We next repeated the experiment using the Jurkat-Raji co-culture system. Once again, we found that the antibody had a net activating effect on T cells (FIG. 5E). At a fixed concentration of 1 ug/ml of anti-CD45/SLAMF6, enhanced T cell activation was seen across increasing doses of SEE added (FIG. 5E). A dose response increase in T cell activation was also seen with increasing levels of the bispecific antibody used, with maximal activation seen at a dose of 10 ug/ml (FIG. 5F). On the other hand, stimulation with anti-SLAMF6 (1 ug/ml) and/or anti-CD45 (1 ug/ml) did not significantly enhance the T cell activation as compared to the addition of SEE alone (FIG. 5F).

Receptor ligation by the anti-CD45/SLAMF6 has two possible mechanisms of clustering these two receptors: the binding of SLAMF6 and CD45 in trans, between T cells and APC, or in cis, along the T cell surface. In an attempt to better understand how anti-CD45/SLAMF6 resulted in T cell activation despite decreased enrichment of SLAMF6 in the IS, we sought to investigate whether the antibody binds in trans or in cis. Specifically, the aim of this experiment was to compare T cell activation when the anti-CD45/SLAMF6 was added directly into a T and B cells co-culture (trans binding) as compared to adding the anti-CD45/SLAMF6 antibodies to T cells alone (and washing off unbound antibody) prior to co-culturing with B cells (cis binding). In the first condition, SEE was added to the co-culture to initiate the Jurkat-Raji crosslinking, and this was followed by the addition of the bispecific antibody, thereby preferentially inducing trans binding. Alternatively, in the second condition, we pretreated the Jurkat cells with the bispecific antibody before co-culturing, followed by a wash and subsequent addition of SEE to allow for Jurkat-Raji interaction (FIG. 5G). IL-2 levels were increased when anti-CD45/SLAMF6 was added to the Jurkat-Raji co-culture system (trans binding). However, the IL-2 levels were still further increased when the anti-CD45/SLAMF6 antibody was added to Jurkat T cells before the T and B cell co-culture, thus allowing the antibody to bind in cis. This led us to conclude that the anti-CD45/SLAMF6 antibody function in cis along the T cell surface was predominantly responsible for the IL-2 release, and may explain the increased T cell activation despite hindered SLAMF6 clustering in the IS.

Our data show that while anti-CD45/SLAMF6 reduces SLAMF6 clustering in the synapses, the bispecific antibody still functions to enhance T cell activation and IL-2 release downstream of the TCR signal. We propose that the antibody exerts its function predominantly via binding in cis along the T cell surface.

Co-Stimulation of Primary Mononuclear Cells with Anti-CD45 SLAMF6 Augments T Cell Activation.

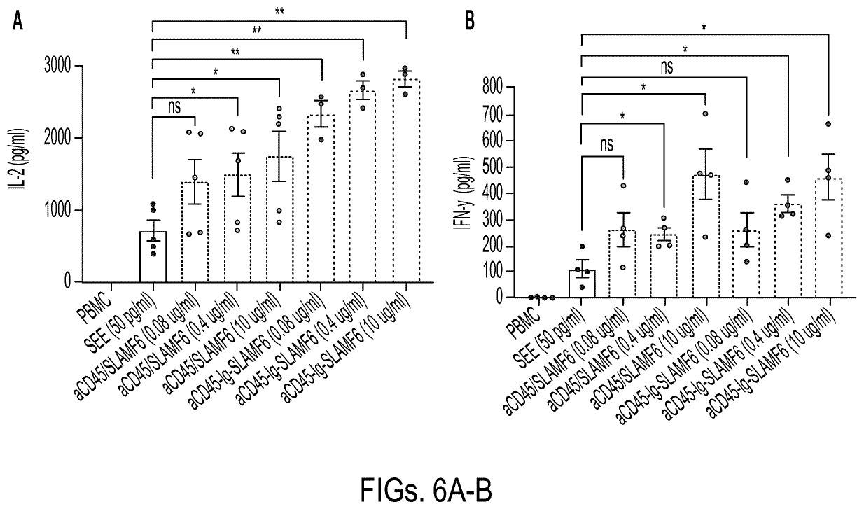

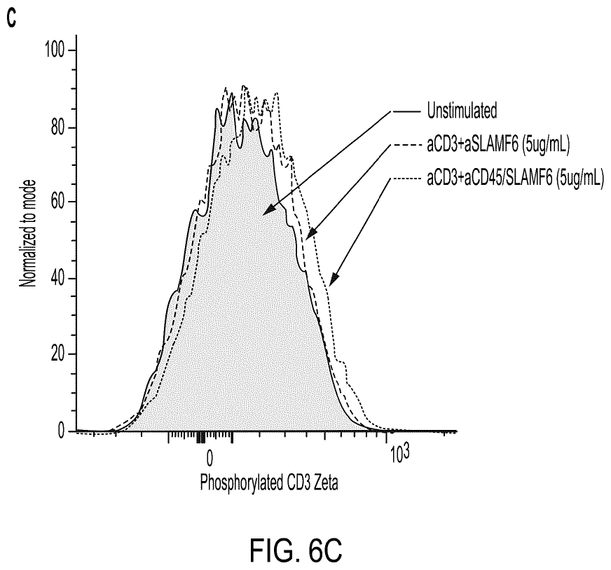

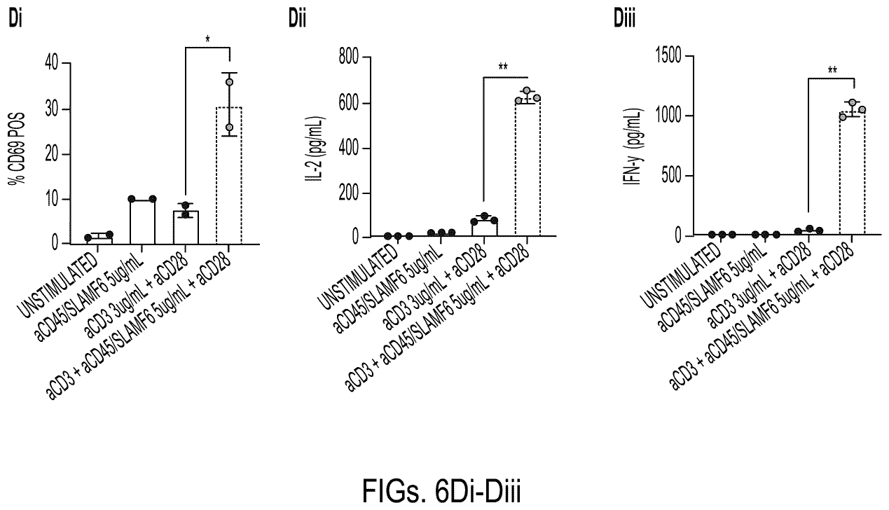

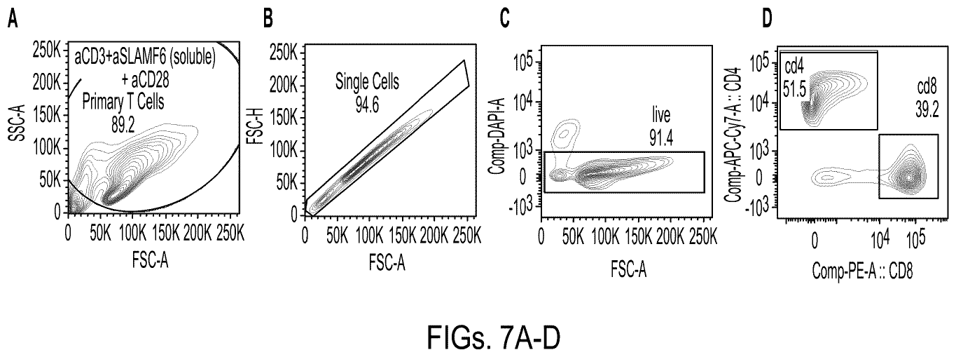

Observing the stimulatory effect of the anti-CD45/SLAMF6 in an isolated Jurkat T cell system, as well as a Jurkat-Raji co-culture, we next sought to evaluate the performance of the bispecific antibody in a more complex microenvironment of mixed primary immune cells. For this, to best mimic the in vivo microenvironment, we chose an ex vivo human peripheral blood mononuclear cells (PBMC) assay. PBMC from healthy donors were activated by the addition of SEE (50 pg/ml). Increasing doses of monovalent anti-CD45/SLAMF6 or bivalent anti-CD45-Ig-SLAMF6 antibodies were added. In presence of SEE, the addition of the bispecific antibody resulted in increased IL-2 (FIG. 6A) and IFN-γ (FIG. 6B) release. We observed higher levels of IL-2 release with the bivalent antibody, indicative of its ability to bind more antigen sites resulting in greater receptor clustering as compared to the monovalent anti-CD45/SLAMF6 antibodies.

SLAMF6 is expressed on a wide variety of immune cells, and we could not be sure if the T cell activation in the PBMC assay was a result of anti-CD45/SLAMF6 binding directly to the T cells, or indirectly as a result of APC activation. This led us to evaluate the effect of anti-CD45/SLAMF6 on primary T cells in culture. Following a brief 5 minutes stimulation of primary T cells with anti-CD3 in presence or absence of anti-CD45/SLAMF6 antibodies, a trend towards increased phosphorylation of CD3 zeta chain was detected (FIG. 6C). After a 24 hours incubation under the same stimulation conditions, we observed a significant increase in cell surface CD69 expression (FIG. 6Di) as well IL-2 (FIG. 6Dii) and IFN-γ (FIG. Diii) release.

Thus, we show that anti-CD45/SLAMF6 bispecific antibody is able to enhance T cell activation in an isolated Jurkat-Raji co-culture, in primary T cells, and in a more complex PBMC microenvironment, where it functions to directly activate T cells. This suggests that, in primed T cells, this bispecific antibody has an excitatory effect on T cell activation.

T cell activation is a result of a primary TCR-CD3 complex signal that is fine-tuned by secondary co-stimulatory or co-inhibitory signals transmitted by cell surface receptors that cluster together with the TCR in the contact zones of the immunologic synapse. SLAMF6 is a transmembrane T cell co-receptor with contradictory reports in the literature as to whether its net effect is to activate or inhibit the TCR signal. Yigit, B., et al., SLAMF6 as a Regulator of Exhausted CD8(+) T Cells in Cancer. Cancer Immunol Res, 2019. 7(9): p. 1485-1496; Hajaj, E., et al., SLAMF6 deficiency augments tumor killing and skews toward an effector phenotype revealing it as a novel T cell checkpoint. Elife, 2020. 9. In this work we show that the spatial compartmentalization of SLAMF6 with respect to the TCR-CD3 complex in the IS contributes to whether the co-receptor enhances or dampens TCR signal transduction. Specifically, T cell proliferation is inhibited, and there is a trend towards decreased IL-2 release, when the SLAMF6 receptor is engaged but physically removed from the CD3 receptor. On the other hand, when the clustering of SLAMF6 with CD3 is enforced by means of conjugated beads, cross-linking or bispecific anti-CD3/SLAMF6 antibody, T cell activation is significantly enhanced compared to stimulation with anti-CD3 or anti-CD3+anti-SLAMF6 combinations. Thus, the spatial distribution of the SLAMF6 receptors in reference to the TCR-CD3 mediates the net signaling effect following receptor ligation. Similar spatial reorganization of co-receptors affecting T cell function has recently been described for another T cell co-receptor, LAG3. Within minutes following LAG3 ligation, the receptor was noted to colocalize with the TCR-CD3 and migrate to the IS, where it exerted its inhibitory function by causing dissociation of LCK from the TCR, dampening TCR activation. Guy, C. M. M. D. C., P. C.; Temirov, J.; Vignali, K. M.; Liu, X; Zhang, H; Kriwacki, R; Bruchez M. P.; Watkins, S. C.; Workman, C. J.; Vignali, D. A. A., LAG3 associates with TCR-CD3 complexes and suppresses signaling by driving co-receptor-Lck dissociation. Nat Immunol, 2022.

The spatial clustering of receptors is essential as it allows for further interaction of the cytoplasmic tails and their associated signaling molecules, propagating the activation signal forward. It is possible that SLAMF6 and/or TCR ligation results in cytoplasmic assembly of signaling units that result in directed movement of the co-receptor along the cell surface. However, our group has previously shown that while vital for T cell activation, the SLAMF6 ectodomain is not necessary to initiate SLAMF6 trafficking to the synapse, suggesting migration signals beyond the ectodomain activation are responsible. Dragovich, et al. (2019). In the case of SLAMF6, following antigen engagement, the SLAM associated protein (SAP) binds to the SLAMF6 intracellular tail and promotes phosphorylation of SLAMF6's ITSM domain by the SRC kinases LCK and FYN. Both SAP and the SRC kinases, in turn, bridge the activation with the TCR-CD3 complex where LCK phosphorylates and activates ZAP70, initiating TCR activation. In our analysis of SLAMF6 interactome, we confirm the association of a number of TCR signaling proteins with the activated SLAMF6 receptor. The clustering of SLAMF6 with the CD3 in the IS allows the intracellular signaling proteins downstream of SLAMF6 to bridge and amplify the TCR signal. On the other hand, we found that when SLAMF6 is removed from the synapse, the intracellular signaling proteins identified in the SLAMF6 interactome are the same as when SLAMF6 is in the IS. We therefore propose that SLAMF6 is able to “steal” signaling proteins, many of which (i.e., LCK) are required in the TCR activation pathway. In such instances of SLAMF6 being removed from the CD3, the net effect is inhibition following SLAMF6 ligation. The concept of inhibiting TCR signaling be removing LCK from the tail of the CD3 complex has been reported recently for the inhibitory receptor LAG3. Guy, et al. (2022). We conclude that the clustering or removing of SLAMF6 from the TCR-CD3 complex modulate cell activation by either contributing or removing key signaling proteins essential for TCR activation.

Bispecific antibodies are designed to simultaneously recognize two different antigen binding sites. Trans binding can bridge cells together, while cis binding can bring the antigen expressing moieties closer together in physical space along the cell surface membrane. In cancer immunology, some bispecific antibodies bind in trans to simultaneously engage tumor-associated antigens and CD3, augmenting the anti-tumor response by physically linking tumor cells to T cells. Kantarjian, H., et al., Blinatumomab versus Chemotherapy for Advanced Acute Lymphoblastic Leukemia. N Engl J Med, 2017. 376(9): p. 836-847; Berek, J. S., et al., Catumaxomab for the treatment of malignant ascites inpatients with chemotherapy-refractory ovarian cancer: a phase II study. Int J Gynecol Cancer, 2014. 24(9): p. 1583-9. Alternatively, other bispecific antibodies bind in cis; an example of which is the targeting of PD-1 with the CD45 phosphatase, resulting in reduced PD-1 phosphorylation and enhanced T cell activity. Fernandes, R. A., et al., Immune receptor inhibition through enforced phosphatase recruitment. Nature, 2020. 586(7831): p. 779-784. In this work we bioengineered two bispecific antibodies, anti-CD3/SLAMF6 and anti-CD45/SLAMF6, targeting the CD3 and the SLAMF6 receptors, and CD45 and SLAMF6 receptors respectively. We hypothesized that anti-CD3/SLAMF6, by bridging the two receptors together, would enhance T cell activation. Indeed, we found that stimulation of T cells with anti-CD3/SLAMF6 augments activation to a greater extent than either anti-CD3 or anti-SLAMF6 alone or in an admixed combination. Thus, we suggest that antibodies targeting in cis clustering of T cell co-receptors such as SLAMF6 may have immunotherapeutic potential to regulate T cell function in disease. Furthermore, while bispecific antibodies that activate CD3, such as anti-CD3/SLAMF6, are fraught with the risk of non-specific T cell activation and adverse inflammatory immune related effects, bispecific antibodies targeting co-receptor function, such as anti-CD45/SLAMF6, would be expected to minimize this risk. Specifically, unlike anti-CD3/SLAMF6, anti-CD45/SLAMF6 would not be expected to activate resting T cells in the absence of TCR engagement and may therefore be a safer therapeutic intervention to target only the activated T cells, minimizing off-target effects.

Similarly, and somewhat unexpectedly, we found that anti-CD45-SLAMF6 antibody can also enhance T cell activation. CD45 is a transmembrane protein tyrosine phosphatase expressed on most hematopoietic cells. CD45 localization on T cells, while critical for cell activation, is such that it is excluded from the IS due to its bulky steric hindrance. Cordoba, et al. (2016); Chang, et al. (2013). Additionally, as mentioned above, bispecific antibodies in development rely on the CD45 phosphatase to co-localize and dephosphorylate the associated co-receptors through in cis interaction. Fernandes, et al., (2020). We thus hypothesized that by tethering the SLAMF6 to the large and bulky ectodomain of CD45 phosphatase, we could exclude SLAMF6 from the immunologic synapse due to steric hindrance, resulting in net inhibition of T cell function. Cordoba, et al. (2016); Chang, et al. (2013). Contrary to our expectation, stimulation with anti-CD45/SLAMF6 enhanced rather than inhibited T cell activation. We repeatedly found that stimulation with anti-CD45/SLAMF6 resulted in net activation effect on T cell function. Several explanations exist to explain our findings. First, CD45 is critical for T cell survival and CD45 deficient mice have a block in T cell development that is attributed to the ability of CD45 to dephosphorylate a negative regulatory tyrosine on LCK. Thus, the CD45 functions to activate LCK kinase, priming it for recruitment to the SLAMF6 and TCR, further propagating the activation signal. Rheinlander, A., B. Schraven, and U. Bommhardt, CD45 in human physiology and clinical medicine. Immunol Lett, 2018. 196: p. 22-32; Philipsen, L., et al., De novo phosphorylation and conformational opening of the tyrosine kinase Lck act in concert to initiate T cell receptor signaling. Sci Signal, 2017. 10(462); Saunders, A. E. and P. Johnson, Modulation of immune cell signalling by the leukocyte common tyrosine phosphatase, CD45. Cell Signal, 2010. 22(3): p. 339-48. Therefore, the proximity of CD45 to SLAMF6 that was achieved with our bispecific antibody may have resulted in augmented LCK activation, promoting SLAMF6 phosphorylation and overall T cell activation. Second, microscopy imaging shows that while SLAMF6 clustering at the IS is reduced following treatment with anti-CD45/SLAMF6 in the mature synapse, it was not completely abolished. Indeed, initial recruitment of CD45 to the IS is required for cell signal propagation, with expulsion of CD45 due to steric hindrance occurring only subsequently to its initial recruitment. Rheinlander, et al. (2018). Finally, it is possible that while we intended our bispecific antibody to bind in cis, some degree of in trans binding occurred, promoting cell clustering and net activation.

Taken together, we show that SLAMF6 activity is dependent on its localization along the cell surface with respect to the TCR site. Activation of SLAMF6 in proximity to the CD3 shows that SLAMF6 is an activating T cell co-receptor. SLAMF6 interacts with a number of proximal TCR signaling proteins, suggesting translocation to the TCR site following receptor ligation. Enhanced T cell activity can be achieved by stimulating with an anti-CD3/SLAMF6 bispecific antibody, with a dose specific response seen in our assays. On the other hand, when SLAMF6 is removed from the TCR site, it is able to “steal” with it many of the essential proximal signaling proteins that are required for TCR signal propagation. Thus, a neutralizing, and sometimes inhibiting, effect can be seen. We conclude that spatial localization of T cell co-receptors along the T cell surface may be a novel mechanism to target T cell activity in disease.

Our work has several limitations. We show that despite differences in cell activation, mass spectrometry analysis of the SLAMF6 interactome when clustered with CD3 as compared to when it is removed from CD3 is not remarkably different. We explain this by a “steal” phenomenon where SLAMF6 localization away from the CD3 removes essential signaling proteins away from the CD3, dampening T cell signaling. A limitation to our explanation is that we were not able to detect CD3 in our IP, thus we could not prove differential CD3 association between the two conditions. Another limitation of the IP is that we did not have results for protein phosphorylation states. Yet another limitation is that while we show that the synthesized bispecific antibodies in this work are functional with the promise to enhance T cell activity, we were not able to achieve T cell inhibition by removing SLAMF6 from the IS. While we believe that this was a result of in cis interaction between CD45 and SLAMF6, the exact mechanism remains to be evaluated in future works. Finally, we show functional activity of the synthesized antibodies in biochemical, co-culture and PBMC experiments, we were not able to evaluate the antibody function in animal models. Future work will focus on obtaining humanized mice and/or synthesizing mouse-specific antibodies to better evaluate the therapeutic potential of bispecific SLAMF6 activating antibodies in tumor immunology.

### Materials and Methods

### General Reagents

RPMI medium 1640, DMEM, PBS and FBS were purchased from Life Technologies. Staphylococcus Enterotoxin E (SEE) was acquired from Toxin Technology. BCA protein assay kit was purchased from Thermo Scientific (#23227).

### Peripheral Blood Mononuclear Cell (PBMC) Isolation

10-15 ml of whole blood was collected into EDTA tubes from healthy volunteers who provided informed consent (IRB-AAAB3287). Mononuclear cells were isolated using Lymphoprep density gradient centrifugation (STEMCELL Technologies).

### Cells

Jurkat T cells and Raji B cells were obtained from the American Type Culture Collection (ATCC). The cells were cultured in RPMI 1640 medium supplemented with 10% FBS and 100 U/ml penicillin and streptomycin. Primary CD3⁺ T cells were isolated from PBMCs using the EasySep Human T Cell Isolation Kit (#17951) and were grown in RPMI 1640 medium supplemented with 10% FBS and 100 U/ml penicillin and streptomycin, non-essential amino acids (2 nM) and L-glutamine (2 mM). HEK293T cells were obtained from ATCC and cultured in DMEM media supplemented with 10% FBS and 100 U/ml penicillin and streptomycin.

### SLAMF6 Knockout Jurkat T Cells

SLAMF6 was knocked out (KO) in Jurkat T cells by CRISPR-Cas9 using two of the lentiCRISPR v2 plasmid purchased from GeneScript. Two sets of lentiviral particles were generated as before each set containing one of the different lentiCRISPR v2 plasmids. Viral particles were transduced by centrifugation and cells were selected with puromycin.

### OFPSpark-CD45 and GFP-SLAMF6 Expressing Jurkat T Cells

SLAMF6-GFP fusion expression constructs were generated through PCR amplification and cloning of SLAMF6 (DNASU #HsCD00446754) into pEGFP-N1 vector (Invitrogen). OFPSpark-CD45 DNA constructs were obtained from Sino Biologics (#HG10086-ACR). DNA expression constructs were introduced into the SLAMF6 KO Jurkat T cells by nucleofection according to the optimization protocol (Lonza Nucleofector II).

### V5-SLAMF6 Stable Jurkat T Cell Line

V5-SLAMF6 expression construct was purchased from DNASU (#HsCD00938682). Plasmid DNA was isolated using the maxi plasmid purification kit (Qiagen). For lentiviral production, plasmid DNA expressing V5 tagged SLAMF6 was co-transfected with pMD2G envelope and psPAX2 packaging plasmids in HEK293T cells using SuperFect transfection reagent (Qiagen). Two million Jurkat T cells were lentivirus transduced by spinoculation at 800×g for 30 min at 32° C. Blasticidin selection was used for the generation of V5-SLAMF6 expressing Jurkat clones. SLAMF6 expression was confirmed by flow cytometry analysis using PE-fluorescent anti-SLAMF6 antibody staining (BioLegend #317207).

### Antibodies and Stimulation

Anti-CD3 (UCHT1, BioLegend #300465) and anti-SLAMF6 (BioLegend #317202) antibodies were used for stimulation. For immobilized stimulation, plates were coated with anti-CD3 (3 ug/ml, unless otherwise indicated) and anti-SLAMF6 (5 ug/ml, unless otherwise indicated); for soluble stimulation the antibodies were added in suspension at the same concentration. Antibody-coupled Dynabeads (Invitrogen #14311D) were created according to manufacturer's protocol. Anti-CD3/IgG Isotype, anti-SLAMF6/IgG Iso and anti-CD3/anti-SLAMF6 conjugated beads were used for stimulation in a final suspension of 0.1-0.2 mg beads per ml.

Bispecific antibodies used in this study were made by a rational structure-guided approach resulted in a set of substitutions that were reported to lead to over 90% heterodimers with a high thermal stability. The single-chain fragment variable (scFv) of the anti-SLAMF6 Ab and the anti-CD45 Ab were linked to the fragment crystallizable region (Fc) of the two heavy chains included T350V, T366L, K392L, and T394W mutations in the first Fc chain and T350V, L351Y, F405A, and Y407V mutations in the second Fc chain. The bispecific antibodies were generated by subcloning the binding domains of OKT3, SLAMF6, and CD45 into pVax1 vector (Thermo #V26020) using HindIII and Xba1 restriction sites (Sequences are included in Sup. Tab. 1). Expi293 cells (Thermo #A1435101) were grown with serum free expression medium until confluency on 37° C. CO₂ shaker at 110 rpm. 200 million cells were transfected with Gibco expifectamime transfection kit (Thermo #A14524) following manufacture's recommendations. On day 5 post transfection, supernatant was collected and antibodies were separated using protein A agarose beads (Thermo #20333) and elution buffer (Thermo #21004). Antibodies were supplemented with 1M HEPS buffer and concentration was determined by OD measurement at 280 nm and by running PAGE gel against lug of BSA as a control.

### Cell Proliferation Assay

Jurkat T cells were activated with soluble anti-CD3 or anti-CD3+anti-SLAMF6 and cultured for 72 hours. Number of cells was assessed by automated counting (Invitrogen Countess II) in the presence of trypan blue. Primary CD3⁺ T cells were isolated, stained with 1 μM of carboxyfluorescein succinimidyl ester (CFSE; BioLegend #79898) then activated with immobilized anti-CD3 or anti-CD3/anti-SLAMF6 for 120 hours. Cells were then assayed for proliferation via flow cytometry after a period of five days under stimulatory conditions.

### Flow Cytometry Analysis

At the completion of stimulation, cells were collected, and surface stained for CD4-FITC (BL #300506) or CD4-AF700 (BL #300526, CD8-BV605 (BL #301040), CD69-BV421 (BL #310930), CD25-FITC (BL #302604), PD1-BV711 (BL #329928), CD45-BV421 (BL #304129) and CCR7-APC (BL #353213). Intracellular staining for IL-2-PE (BL #500307) was performing following fixation (BL #420801) and permeabilization (BL #421002). Intracellular staining for phosphorylated CD3 zeta-AF647 (BD #558489) was performed following fixation and above and permeabilization (BL #76344). Flow cytometry was performed on the BD LSRII and analyzed with FlowJo v10.8.1 software.

### Confocal Microscopy

To evaluate conjugate formation by confocal microscopy, Jurkat T cells were transfected with GFP-SLAMF6 and/or mCherry-LifeAct-7 (AddGene #54491) expressing constructs. Raji B cells were pre-stained with CellTrace Far Red dye (Invitrogen #C34572, 1:1000 dilution for 20 minutes in PBS). In some conditions, 1×10⁶ Jurkat T cells were pre-treated with either anti-SLAMF6 or anti-CD45/SLAMF6 as per the experimental design. 1×10⁶ Raji B cells were coated with 2 mg/ml SEE in FCS-free RPMI for 2 hours. Next, 2-3×10⁵ in 100 uL Jurkat T cells were mixed with 2-3×10⁵ in 100 uL Raji B cells. The coculture was placed on a glass bottom culture dish (MatTek Corporation) and rested for 15 min to allow conjugates to form. Subsequently, confocal images from each stimulation were acquired on Zeiss LSM 900 confocal microscope and analyzed with ZEN Blue software.

### The Enzyme-Linked Immunosorbent Assay (ELISA)

To determine the concentration of secreted proteins after stimulation, human IFN-γ (BioLegend #430101) and human IL-2 (BioLegend #431801) detection kits were used. To quantify anti-CD45 and anti-SLAMF6 antibody binding, polystyrene high binding microplates (Corning) were coated with immobilized CD45 (SinoBiological #14197-H08H) or SLAMF6 (SinoBiological #11945-H08H) recombinant ectodomain proteins, respectively. To quantify anti-CD3 and/or anti-SLAMF6 antibody binding to CD3, immobilized SLAMF6 KO Jurkat T cell lysates were used as antigen bait. Primary antibody binding was detected using a secondary HRP goat antibody recognizing human Fc (Sino-Biologic #SSA001-200).

### Immunoprecipitation

Jurkat T cells expressing V5 tagged SLAMF6 were stimulated with immobilized anti-CD3 and anti-SLAMF6 as described above. 30×10⁶ Jurkat T cells from each condition were lysed in an IP lysis buffer (1% NP-40, 25 mMTris pH 8.0, 150 mM NaCl, 1 mM EDTA, 5% Glycerol) containing protease and phosphatase inhibitors. Cell lysates were incubated with anti-V5 monoclonal antibody coupled to agarose beads to enrich V5-tagged SLAMF6, according to the manufacturer's protocols (MBL #PM003-8). Pull-down lysates were separated by Tris-glycine gels and submitted for mass spectrometry analysis at Quantitative Proteomics and Metabolomics Center at Columbia University and NYU Langone's Proteomics Laboratory. Proteins identified were analyzed in the context of known signaling pathways, identified using public databases (uniport.org and reactome.org). Protein-protein interaction were explored using STRING analysis (https://string-db.org/).

### Statistics

Values are reported as mean±SD or median±95% CI. Statistical analyses were performed using Student's paired t-test for normally distributed data and Mann-Whitney U test for skewed data. All statistical analyses were performed using GraphPad Prism (version 8.0). Illustrations were created using BioRender.

### Example 2—T Cell Stimulation with Anti-CD43/SLAMF6 Bispecific Antibody Enhances T Cell Activity

An anti-CD43/SLAMF6 bispecific antibody as a fusion immunoglobulin of CD43-IgG-hole (SEQ ID NO: 7) and SLAMF6-IgG-knob (SEQ ID NO: 9) was also designed. The anti-CD43/SLAMF6 bispecific antibody was also validated to bind to its intended targets (data not shown). Experiments evaluating the effect of the anti-CD43/SLAMF6 bispecific antibody on localization suggested that SLAMF6 was excluded from the IS in mature synapses. Further experiments showed that while anti-CD3/SLAMF6 reduces SLAMF6 clustering in the synapses, the bispecific antibody still functions to enhance T cell activation and IL-2 release downstream of the TCR signal.

### Example 3—In Vivo Study

In some embodiments, experiments are designed to test these bispecific antibodies in vivo using syngeneic tumor model, in comparison to anti-SLAMF6 monoclonal antibodies.

This example describes animal studies with anti-CD3/SLAMF6, anti-CD45/SLAMF6, and anti-CD43/SLAMF6 bispecific antibodies described herein.

The anti-CD3/SLAMF6, anti-CD45/SLAMF6, and anti-CD43/SLAMF6 bispecific antibodies described herein, expressed as recombinant proteins, are analyzed in animal studies include mouse models.

The anti-CD3/SLAMF6, anti-CD45/SLAMF6, and anti-CD43/SLAMF6 bispecific antibodies described herein, will be produced under cGMP conditions as a recombinant protein for use in Phase I clinical trials.

The devices, systems, and methods disclosed herein are not to be limited in scope to the specific embodiments described herein. Indeed, various modifications of the devices, systems, and methods in addition to those described will become apparent to those of skill in the art from the foregoing description.

### Example 4—In Vitro Binding of Anti-CD45 Abs

FIG. 11A-B show binding curves to human-CD45RO-ECD-His-coated plates was quantified by ELISA. FIG. 11A shows binding curves for anti-CD45 antibody clones 023, 026, 027, and 028, CD45RO Monoclonal Antibody (UCHL1) (eBioscience™), anti-HEL-human IgG1 isotype control, and blank. FIG. 11B shows binding curves for anti-CD45 antibody clones 031, and 042, CD45RO Monoclonal Antibody (UCHL1) (eBioscience™), anti-HEL-human IgG1 isotype control, and blank. EC50 values were calculated with GraphPad Prism (v10.2.1). Table 1 shows EC50 values for or anti-CD45 antibody clones 023, 026, 027, and 028, CD45RO Monoclonal Antibody (UCHL1) (eBioscience™), and anti-HEL-human IgG1 isotype control. Table 2 shows EC50 values for or anti-CD45 antibody clones 031 and 042, CD45RO Monoclonal Antibody (UCHL1) (eBioscience™), and anti-HEL-human IgG1 isotype control. These results show that the anti-CD45 antibody clones are capable of binding to CD45, with lower EC₅₀ values when compared to the commercially-available CD45RO Monoclonal Antibody.

TABLE 1

| Antibody | EC₅₀(nM) |
|---|---|
| Clone 023 | 0.2193 |
| Clone 026 | 0.1326 |
| Clone 027 | 0.07336 |
| Clone 028 | 0.03192 |
| CD45RO Monoclonal Antibody | 0.3282 |
| (UCHL1) (eBioscience ™) | |
| anti-HEL-human IgG1 isotype control | No Binding |

TABLE 2

| Antibody | EC₅₀(nM) |
|---|---|
| Clone 031 | 0.1394 |
| Clone 042 | 0.1439 |
| CD45RO Monoclonal Antibody | 0.5439 |
| (UCHL1) (eBioscience ™) | |
| anti-HEL-human IgG1 isotype control | No Binding |

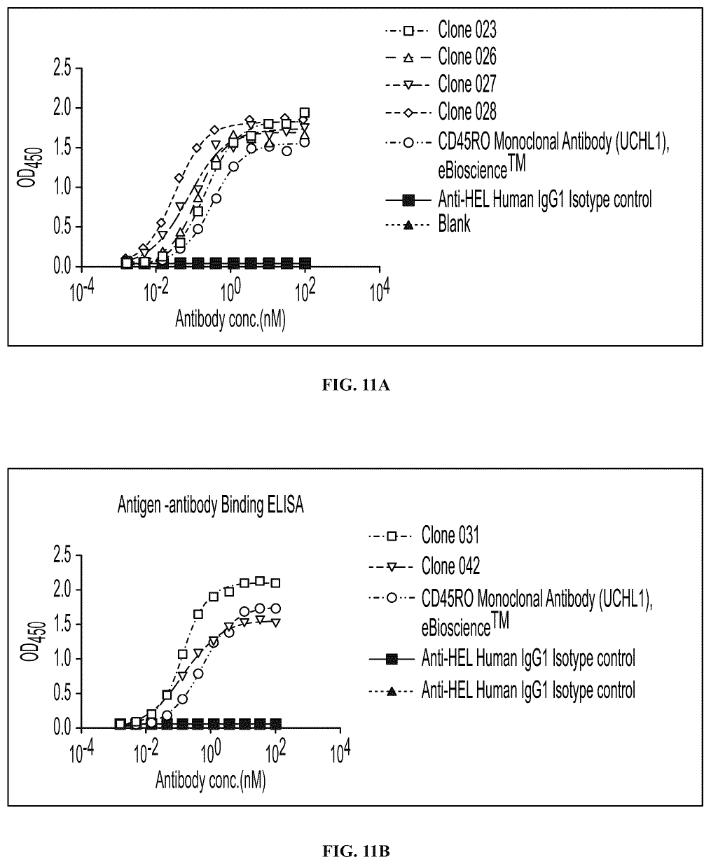

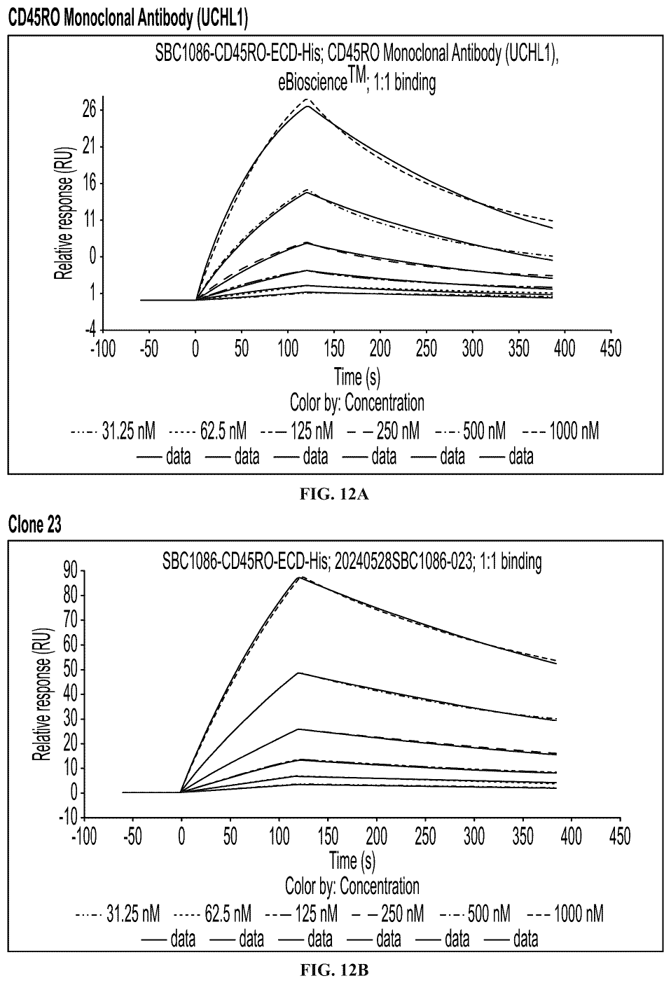

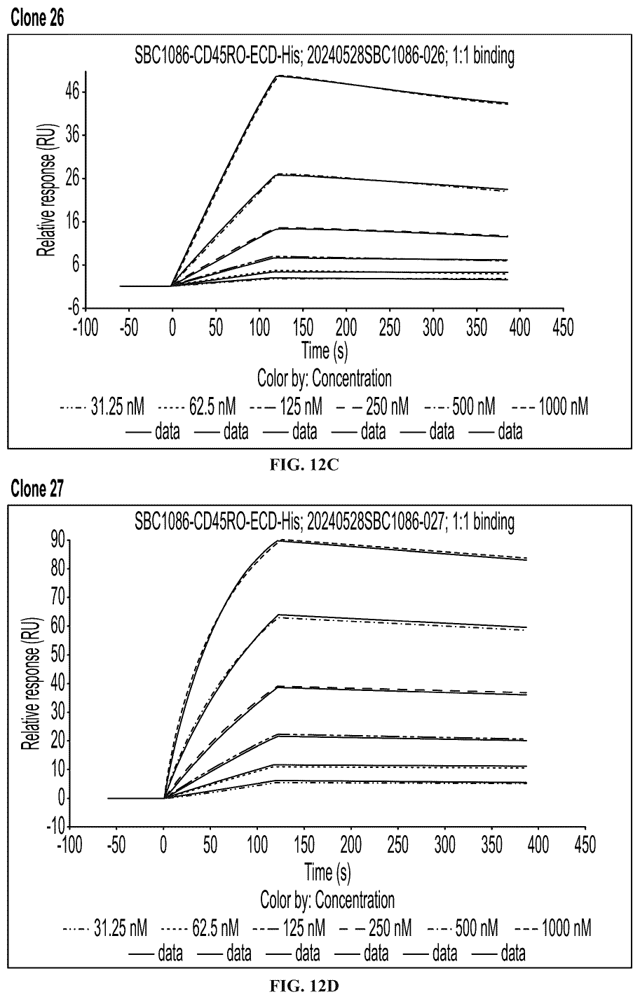

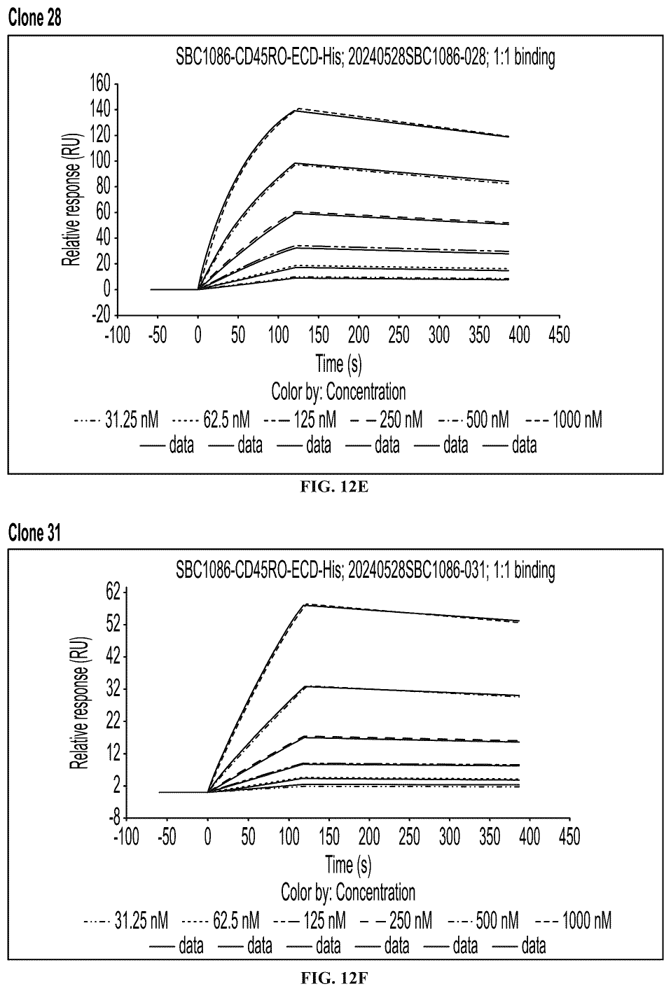

FIG. 12A-E shows SRP analysis of binding of anti-CD45 antibodies to captured human-CD45RO-ECD-His. FIG. 12A shows binding of CD45RO Monoclonal Antibody (UCHL1) to captured human-CD45RO-ECD-His. FIG. 12B shows binding of anti-CD45 antibody clone 23 to captured human-CD45RO-ECD-His. FIG. 12C shows binding of anti-CD45 antibody clone 26 to captured human-CD45RO-ECD-His. FIG. 12D shows binding of anti-CD45 antibody clone 27 to captured human-CD45RO-ECD-His. FIG. 12E shows binding of anti-CD45 antibody clone 28 to captured human-CD45RO-ECD-His. FIG. 12F shows binding of anti-CD45 antibody clone 31 to captured human-CD45RO-ECD-His. Table 3 shows the binding ka (1/Ms), kd (1/s), and KD (M) for CD45RO Monoclonal Antibody (UCHL1), and anti-CD45 antibody clones 023, 026, 027, 028, and 031.

TABLE 3

| Ligand | 1:1 binding | kd(1/s) | KD(M) |
|---|---|---|---|
| CD45RO Monoclonal Antibody | 8.61E+03 | 3.73E−03 | 4.33E−07 |
| Clone 023 | 4.06E+03 | 1.95E−03 | 4.79E−07 |
| Clone 026 | 2.03E+03 | 5.24E−04 | 2.57E−07 |
| Clone 027 | 7.58E+04 | 2.82E−04 | 3.72E−09 |
| Clone 023 | 1.48E+04 | 5.91E−04 | 3.99E−08 |
| Clone 031 | 4.22E+03 | 3.35E−04 | 7.95E−08 |

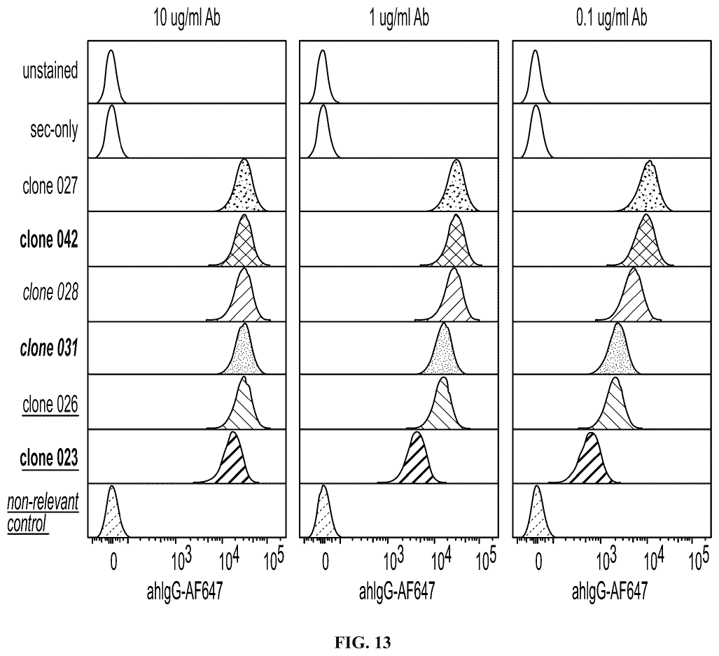

FIG. 13 shows binding of anti-CD45 antibodies to cell-expressed CD45 in Jurkat cells by flow cytometry. FIG. 13 shows binding for anti-CD45 antibody clones 027, 042, 028, 031, 026, and 023 compared to unstained, sec-only, and non-relevant control. Wild type Jurkat T cells were incubated with either 10 μg/ml (left), 1 μg/ml (middle), or 0.1 μg/ml (right) of each anti-CD45 clone on ice for 30 minutes, washed twice, and then incubated for 30 minutes on ice with a fluorescent anti-human-Fc secondary antibody. Following two additional washes, cells were analyzed on a BD LSRFortessa™ flow cytometer. These results show that anti-CD45 antibodies bind to cell-expressed CD45 in Jurkat cells.

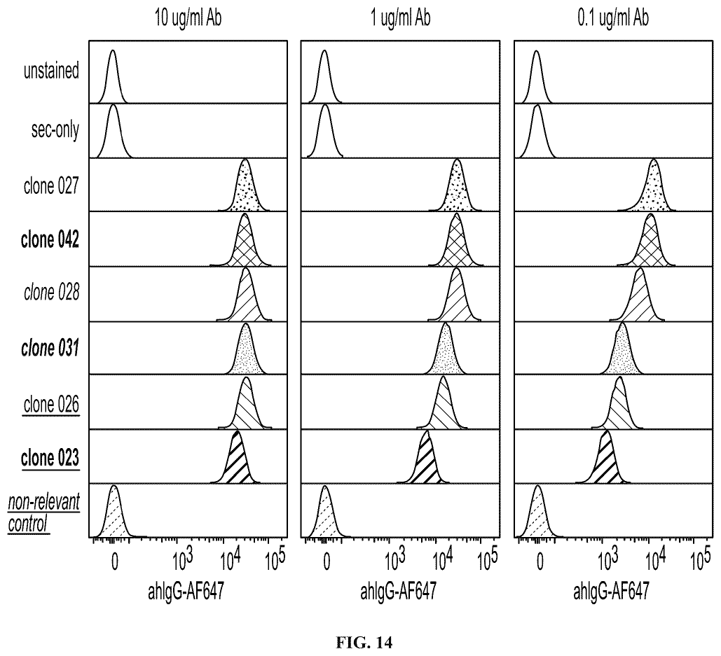

FIG. 14 shows binding of anti-CD45 antibodies to cell-expressed CD45 in Raji cells by flow cytometry. FIG. 14 shows binding for anti-CD45 antibody clones 027, 042, 028, 031, 026, and 023 compared to unstained, sec-only, and non-relevant control. Wild type Raji B cells were incubated with either 10 μg/ml (left), 1 μg/ml (middle), or 0.1 μg/ml (right) of each anti-CD45 clone on ice for 30 minutes, washed twice, and then incubated for 30 minutes on ice with a fluorescent anti-human-Fc secondary antibody. Following two additional washes, cells were analyzed on a BD LSRFortessa™ flow cytometer. These results show that anti-CD45 antibodies bind to cell-expressed CD45 in Raji cells.

What is claimed is:

1. A bispecific antibody or a fragment thereof, comprising:
a first arm comprising a first variable heavy chain domain and a first variable light chain domain, wherein a portion of the first arm is capable of binding to a portion of a CD3 protein, a portion of a CD45 protein, or a portion of a CD43 protein; and
a second arm comprising a second variable heavy chain domain and a second variable light chain domain, wherein a portion of the second arm is capable of binding to a portion of a SLAMF6 protein.

2. The bispecific antibody of claim 1, wherein the first arm is encoded by a first polypeptide chain and the second arm is encoded by a second polypeptide chain that associate together.

3. The bispecific antibody of claim 1, wherein the first arm or the second arm comprises a glycine-serine linker between the first variable heavy domain and first variable light chain domain.

4. The bispecific antibody of claim 2, wherein the first and second arms each further comprise a fragment, crystallizable (Fc) region.

5. The bispecific antibody of claim 4, wherein the Fc region of the first arm comprises knob mutations and the Fc region of the second arm comprise hole mutations, or vice versa.

6. The bispecific antibody of claim 1, wherein the bispecific antibody is bivalent.

7. The bispecific antibody of claim 1, wherein the first arm and second arm are encoded on a first polypeptide chain.

8. The bispecific antibody of claim 2, wherein the first polypeptide chain further comprises:
a third arm comprising a third variable heavy chain domain and a third variable light chain domain that is the same as the first variable heavy chain domain and first variable light chain domain; and
wherein the second polypeptide chain further comprises:
a fourth arm comprising a fourth variable heavy chain domain and a fourth variable light chain domain, that is the same as the second variable heavy chain domain and second variable light chain domain.

9. The bispecific antibody of claim 8, wherein the first arm or second arm comprises a glycine-serine linker between the first variable heavy domain and first variable light chain domain.

10. The bispecific antibody of claim 8, wherein the third arm or fourth arm comprises a glycine-serine linker between the third variable heavy domain and third variable light chain domain.

11. The bispecific antibody of claim 8, wherein the first and second polypeptide chains each further comprises a fragment, crystallizable (Fc) region wherein the Fc region of the first polypeptide chain comprises knob mutations and the Fc region of the second polypeptide chain comprises hole mutations, or vice versa. wherein the Fc region of the first polypeptide chain is positioned between the first arm and third arm and the Fc region of the second polypeptide chain is positioned between the second arm and fourth arm.

12. The bispecific antibody of claim 11, wherein the bispecific antibody is tetravalent.

13. The bispecific antibody of claim 1, wherein the first variable heavy chain domain comprises an amino acid sequence of the variable heavy chain portion of SEQ ID NO: 2, wherein the first variable light chain domain comprises an amino acid sequence of the variable light chain portion of SEQ ID NO: 2.

14. The bispecific antibody of claim 1, wherein the first arm comprises SEQ ID NO: 2, wherein the second arm comprises SEQ ID NO: 9.

15. The bispecific antibody of claim 1, wherein the first variable heavy chain domain comprises an amino acid sequence of the variable heavy chain portion of SEQ ID NO: 5, SEQ ID NO: 28, SEQ ID NO: 29, SEQ ID NO: 30, SEQ ID NO: 31, SEQ ID NO: 32, or SEQ ID NO: 33, wherein the first variable light chain domain comprises an amino acid sequence of the variable light chain portion of SEQ ID NO: 5, SEQ ID NO: 28, SEQ ID NO: 29, SEQ ID NO: 30, SEQ ID NO: 31, SEQ ID NO: 32, or SEQ ID NO: 33.

16. The bispecific antibody of claim 1, wherein the first arm comprises SEQ ID NO: 5, SEQ ID NO: 28, SEQ ID NO: 29, SEQ ID NO: 30, SEQ ID NO: 31, SEQ ID NO:
32, or SEQ ID NO: 33, wherein the second arm comprises SEQ ID NO: 9.

17. The bispecific antibody of claim 1, wherein the first variable heavy chain domain comprises an amino acid sequence of the variable heavy chain portion of SEQ ID NO: 7, wherein the first variable light chain domain comprises an amino acid sequence of the variable light chain portion of SEQ ID NO: 7.

18. The bispecific antibody of claim 1, wherein the first arm comprises SEQ ID NO: 7, wherein the second arm comprises SEQ ID NO: 9.

19. The bispecific antibody of claim 1, wherein the second variable heavy chain domain comprises an amino acid sequence of the variable heavy chain portion of SEQ ID NO: 9, wherein the first variable light chain domain comprises an amino acid sequence of the variable light chain portion of SEQ ID NO: 9.

20. The bispecific antibody of claim 1, wherein the first arm comprises an amino acid sequence selected from SEQ ID NO: 2, SEQ ID NO: 5, SEQ ID NO: 28, SEQ ID NO: 29, SEQ ID NO: 30, SEQ ID NO: 31, SEQ ID NO: 32, SEQ ID NO: 33, or SEQ ID NO: 7, and wherein the second arm comprises SEQ ID NO: 9.

21. A tetravalent bispecific antibody or a fragment thereof, comprising:
(I) a first fragment antigen-binding (Fab) region, comprising:
a first arm comprising a first variable heavy chain domain and a first variable light chain domain, wherein a portion of the first arm is capable of binding to a portion of a CD3 protein, a portion of a CD45 protein, or a portion of a CD43 protein; and
a second arm comprising a second variable heavy chain domain and a second variable light chain domain, wherein a portion of the second arm is capable of binding to a portion of a CD3 protein, a portion of a CD45 protein, or a portion of a CD43 protein;
(II) a first fragment, crystallizable (Fc) region;
(III) a second Fab region, comprising:
a third arm comprising a first variable heavy chain domain and a first variable light chain domain, wherein a portion of the first arm is capable of binding to a portion of a SLAMF6 protein; and
a fourth arm comprising a second variable heavy chain domain and a second variable light chain domain, wherein a portion of the second arm is capable of binding to a portion of a SLAMF6 protein; and
(IV) a second Fc region, wherein the first and second Fc regions associated together.

22. The tetravalent bispecific antibody of claim 21, wherein one arm of the first Fab region is N-terminal to the first Fc region and wherein the other arm of the first Fab region is C-terminal to the first Fc region, and wherein the first arm of the second Fab region is N-terminal to the second Fc region and wherein the other arm of the second Fab region is C-terminal to the second Fc region.

23. The tetravalent bispecific antibody of claim 21, wherein one arm of the first Fab region is C-terminal to the first Fc region and wherein one arm of the second Fab region is N-terminal to the first Fc region, and wherein the other arm of the first Fab region is C-terminal to the second Fc region and wherein the other arm of the second Fab region is N-terminal to the second Fc region, or wherein one arm of the first Fab region is N-terminal to the first Fc region and wherein one arm of the second Fab region is C-terminal to the first Fc region, and wherein the other arm of the first Fab region is N-terminal to the second Fc region and wherein the other arm of the second Fab region is C-terminal to the second Fc region.

24. A pharmaceutical composition comprising: the bispecific antibody of claim 1; and a pharmaceutically acceptable carrier.

25. A method of preventing or treating cancer in a subject comprising administering to the subject an effective amount of the composition of claim 24.

26. A method of preventing or treating an autoimmune disease in a subject comprising administering to the subject an effective amount of the composition of claim 24.

27. A kit for generating a bispecific antibody or fragment thereof, the kit comprising one or more vectors comprising a polynucleotide sequence encoding any of the bispecific antibodies of any of claim 1.

28. A kit for generating a tetravalent bispecific antibody or fragment thereof, the kit comprising one or more vectors comprising a polynucleotide sequence encoding any of the tetravalent bispecific antibodies of any of claim 21.

29. A kit for generating a bispecific antibody or fragment thereof, the kit comprising:
a first vector comprising a polynucleotide sequence encoding a first arm of the bispecific antibody or fragment thereof, wherein a portion of the first arm is capable of binding to a portion of a CD3, protein, a portion of a CD45 protein, or a portion of a CD43 protein; and
a second vector comprising a polynucleotide sequence encoding a second arm of the bispecific antibody or fragment thereof, wherein a portion of the second arm is capable of binding to a portion of a SLAMF6 protein.

30. One or more host cells comprising: one or more vectors comprising a polynucleotide sequence encoding any of the bispecific antibodies of claim 1.

31. One or more host cells comprising:
a first vector comprising a polynucleotide sequence encoding a first arm of a bispecific antibody or fragment thereof, wherein a portion of the first arm is capable of binding to a portion of a CD3 protein, a portion of a CD45 protein, or a portion of a CD43 protein; and
a second vector comprising a polynucleotide sequence encoding a second arm of the bispecific antibody or fragment thereof, wherein a portion of the second arm is capable of binding to a portion of a SLAMF6 protein.

32. A method of making a bispecific antibody or fragment thereof comprising:
culturing the one or more host cells under conditions suitable for an expression of the one or more vectors; and
recovering the bispecific antibody or fragment thereof.

33. A composition comprising:
one or more vectors comprising a polynucleotide sequence encoding any of the bispecific antibodies of any of claim 1.

34. A composition comprising:
a first vector comprising a polynucleotide sequence encoding a first arm of the bispecific antibody or fragment thereof, wherein a portion of the first arm is capable of binding to a portion of a CD3 protein, a portion of a CD45 protein, or a portion of a CD43 protein; and
a second vector comprising a polynucleotide sequence encoding a second arm of the bispecific antibody or fragment thereof, wherein a portion of the second arm is capable of binding to a portion of a SLAMF6 protein.

35. A means for binding:
a portion of a CD3 protein, a portion of a CD45 protein, or a portion of a CD43 protein; and
a portion of a SLAMF6 protein.


SEQUENCE LISTING (31 sequences)

SEQ ID NO: 1 - protein, 19 aa, synthetic construct
MGWSCIILFLVATATGVHS

SEQ ID NO: 2 - skipped

SEQ ID NO: 3 - DNA, 57 bp, synthetic construct
  source 1..57: mol_type = other DNA
atgggctggagctgcattatcctgttcctggtggccacagccaccggcgtgcacagc

SEQ ID NO: 4 - skipped

SEQ ID NO: 5 - protein, 478 aa, synthetic construct
EVQLQQSGPELEKPGASVKISCKASGYSFTGYNMNWVKQSNGKSLEWIGNIDPYYGGSDYSQKFLGKATLTVDKSSSTAYMHLKSLTSEDSAVYYCARSNMYDGEYYVMDYWGQGTSVTVSSGGGGSGGGGSGGGGSDIVLTQSPASLAVSLGQRATISCRASESVDNFGVSFMNWFQQKPGQPPKLLIYASSNRGSGVPARFSGSGSGTDFSLNVHPMEEDDAAMYFCQQSKAVPRTFGGGTKLEIKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 6 - protein, 476 aa, synthetic construct
DVQLVESGGGLVQPGGSRKLSCAASGFTFSNFGMHWVRQAPEKGLEWVAYVSRGSTTFYYADTVKGRFTISRDNPKNTLFLQMTSLRSEDTAMYYCARSATATWTMDYWGHGTSVTVSSGGGGSGGGGSGGGGSNIMMTQSPSSLAVSAGEKVTMSCKSSQSVFSSSNHMNYLAWYQQKPGQSPKLLIYWASTRESGVPDRFTGSGSGTDFTLTISSVQAEDLAVYYCHQYLSSWTFGGGSKLEIKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 7 - protein, 471 aa, synthetic construct
QVQLQQSAPELARPGASVRMSCKASGYTFTTYTMNWLTQRPGQGLEWIGYINPSSGYTEYNQKFKDKTSLTADTSSSTAYMQLSSLTSEHSAVYYCARASGYSSWFAYWGQGTLVTVSAGGGGSGGGGSGGGGSNIVMTQTPKFLLVSAGDRVTITCKASQSVNNDVGWYQQKPGQSPKLLIYYASNRYTGVPDRFTGSGYGTDFTFTISTVQAEDLAVYFCQQDYNSPLTFGAGTKLELKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 8 - protein, 472 aa, synthetic construct
QVQLQQSGAELARPGASVKMSCKASGYTFTFYAILWVKQMPGQGLEWIGFINPSSGYTSYNQKFKDKATLTADKSSSTAYMQLSSLTSEDSAVYYCARDYGLDYWGQGTTLAVSSGGGGSGGGGSGGGGSDIVMTQAAPSVPVTPGESVSISCRSSKSLLHSNGNTYLYWFLQRPGQSPQLLIYRMSNLASGVPDRFSGSGSGTAFTLGISRVEAGDVGVYYCMQHLEYPFTFGSGTKLEIKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 9 - protein, 476 aa, synthetic construct
DVQLVESGGGLVQPGGSRKLSCAASGFTFSSFGMHWVRQAPEKGLEWVAYINSGSTTFYYADTVKGRFTISRDNPKNTLFLQMTSLGSEDTAMYYCARSATATWTMDYWGQGTSVTVSSGGGGSGGGGSGGGGSNIMMTQSPSSLAVSAGEKVTMSCKSSQSVFVSSNQKNYLAWYQQKPGQSPKLLIYWASTRESAVPDRFTGSGSGTDFTLTIGSVQAEDLAVYYCHQYLSSWTFGGGTKLEIKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 10 - protein, 473 aa, synthetic construct
EVQLQQSGAELVKPGASVKLSCTASGFNIKDTFMHWVKLRPEQGLEWIGRIDPANGYTKYDPRFQGKATIIADTSSNTAYLQLSSLTSEDTAVYYCASGEYYALDYWGQGTSVTVSSGGGGSGGGGSGGGGSDIVLTQSPASLTVSLGQRATISCRASKSVSTSGYSYMHWYQQKPGQPPKLLIYLASNLESGVPARFSGSGSGTDFTLNIHPVEEEDAATYYCQHNRELPYTFGGGTKLEIKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 11 - DNA, 1434 bp, synthetic construct
  source 1..1434: mol_type = other DNA
gaagtgcagctgcagcagagcggcccggaactggaaaaaccgggcgcgagcgtgaaaattagctgcaaagcgagcggctatagctttaccggctataacatgaactgggtgaaacagagcaacggcaaaagcctggaatggattggcaacattgatccgtattatggcggcagcgattatagccagaaatttctgggcaaagcgaccctgaccgtggataaaagcagcagcaccgcgtatatgcatctgaaaagcctgaccagcgaagatagcgcggtgtattattgcgcgcgcagcaacatgtatgatggcgaatattatgtgatggattattggggccagggcaccagcgtgaccgtgagcagcggcggcggcggcagcggcggcggcggcagcggcggcggcggcagcgatattgtgctgacccagagcccggcgagcctggcggtgagcctgggccagcgcgcgaccattagctgccgcgcgagcgaaagcgtggataactttggcgtgagctttatgaactggtttcagcagaaaccgggccagccgccgaaactgctgatttatgcgagcagcaaccgcggcagcggcgtgccggcgcgctttagcggcagcggcagcggcaccgattttagcctgaacgtgcatccgatggaagaagatgatgcggcgatgtatttttgccagcagagcaaagcggtgccgcgcacctttggcggcggcaccaaactggaaattaaaccgaaaagctgcgataaaacccatacctgcccgccgtgcccggcgccggaatttcagggcggcccgagcgtgtttctgtttccgccgaaaccgaaagataccctgtatattacccgcgaaccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgccaggtgagcaacaaagcgctgccggcgccgattgaaaaaaccattagcaaagcgaaaggccagccgcgcgaaccgcaggtgtatgtgtatccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagctttgcgctggtgagcaaactgaccgtggataaaagccgctggcagcagggcaacgtgtttagctgcagcgtgatgcatgaagcgctgcataaccattatacccagaaaagcctgagcctgagcccgggc

SEQ ID NO: 12 - DNA, 1428 bp, synthetic construct
  source 1..1428: mol_type = other DNA
gatgtgcagctggtggaaagcggcggcggcctggtgcagccgggcggcagccgcaaactgagctgcgcggcgagcggctttacctttagcaactttggcatgcattgggtgcgccaggcgccggaaaaaggcctggaatgggtggcgtatgtgagccgcggcagcaccaccttttattatgcggataccgtgaaaggccgctttaccattagccgcgataacccgaaaaacaccctgtttctgcagatgaccagcctgcgcagcgaagataccgcgatgtattattgcgcgcgcagcgcgaccgcgacctggaccatggattattggggccatggcaccagcgtgaccgtgagcagcggcggcggcggcagcggcggcggcggcagcggcggcggcggcagcaacattatgatgacccagagcccgagcagcctggcggtgagcgcgggcgaaaaagtgaccatgagctgcaaaagcagccagagcgtgtttagcagcagcaaccatatgaactatctggcgtggtatcagcagaaaccgggccagagcccgaaactgctgatttattgggcgagcacccgcgaaagcggcgtgccggatcgctttaccggcagcggcagcggcaccgattttaccctgaccattagcagcgtgcaggcggaagatctggcggtgtattattgccatcagtatctgagcagctggacctttggcggcggcagcaaactggaaattaaaccgaaaagctgcgataaaacccatacctgcccgccgtgcccggcgccggaatttcagggcggcccgagcgtgtttctgtttccgccgaaaccgaaagataccctgtatattacccgcgaaccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgccaggtgagcaacaaagcgctgccggcgccgattgaaaaaaccattagcaaagcgaaaggccagccgcgcgaaccgcaggtgtatgtgtatccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagctttgcgctggtgagcaaactgaccgtggataaaagccgctggcagcagggcaacgtgtttagctgcagcgtgatgcatgaagcgctgcataaccattatacccagaaaagcctgagcctgagcccgggc

SEQ ID NO: 13 - DNA, 1416 bp, synthetic construct
  source 1..1416: mol_type = other DNA
caggtgcagctgcagcagagcggcgcggaactggcgcgcccgggcgcgagcgtgaaaatgagctgcaaagcgagcggctatacctttaccttttatgcgattctgtgggtgaaacagatgccgggccagggcctggaatggattggctttattaacccgagcagcggctataccagctataaccagaaatttaaagataaagcgaccctgaccgcggataaaagcagcagcaccgcgtatatgcagctgagcagcctgaccagcgaagatagcgcggtgtattattgcgcgcgcgattatggcctggattattggggccagggcaccaccctggcggtgagcagcggcggcggcggcagcggcggcggcggcagcggcggcggcggcagcgatattgtgatgacccaggcggcgccgagcgtgccggtgaccccgggcgaaagcgtgagcattagctgccgcagcagcaaaagcctgctgcatagcaacggcaacacctatctgtattggtttctgcagcgcccgggccagagcccgcagctgctgatttatcgcatgagcaacctggcgagcggcgtgccggatcgctttagcggcagcggcagcggcaccgcgtttaccctgggcattagccgcgtggaagcgggcgatgtgggcgtgtattattgcatgcagcatctggaatatccgtttacctttggcagcggcaccaaactggaaattaaaccgaaaagctgcgataaaacccatacctgcccgccgtgcccggcgccggaatttcagggcggcccgagcgtgtttctgtttccgccgaaaccgaaagataccctgtatattacccgcgaaccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgccaggtgagcaacaaagcgctgccggcgccgattgaaaaaaccattagcaaagcgaaaggccagccgcgcgaaccgcaggtgtatgtgtatccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagctttgcgctggtgagcaaactgaccgtggataaaagccgctggcagcagggcaacgtgtttagctgcagcgtgatgcatgaagcgctgcataaccattatacccagaaaagcctgagcctgagcccgggc

SEQ ID NO: 14 - DNA, 1416 bp, synthetic construct; identical to SEQ ID NO: 13
  source 1..1416: mol_type = other DNA

SEQ ID NO: 15 - DNA, 1428 bp, synthetic construct
  source 1..1428: mol_type = other DNA
gatgtgcagctggtggaaagcggcggcggcctggtgcagccgggcggcagccgcaaactgagctgcgcggcgagcggctttacctttagcagctttggcatgcattgggtgcgccaggcgccggaaaaaggcctggaatgggtggcgtatattaacagcggcagcaccaccttttattatgcggataccgtgaaaggccgctttaccattagccgcgataacccgaaaaacaccctgtttctgcagatgaccagcctgggcagcgaagataccgcgatgtattattgcgcgcgcagcgcgaccgcgacctggaccatggattattggggccagggcaccagcgtgaccgtgagcagcggcggcggcggcagcggcggcggcggcagcggcggcggcggcagcaacattatgatgacccagagcccgagcagcctggcggtgagcgcgggcgaaaaagtgaccatgagctgcaaaagcagccagagcgtgtttgtgagcagcaaccagaaaaactatctggcgtggtatcagcagaaaccgggccagagcccgaaactgctgatttattgggcgagcacccgcgaaagcgcggtgccggatcgctttaccggcagcggcagcggcaccgattttaccctgaccattggcagcgtgcaggcggaagatctggcggtgtattattgccatcagtatctgagcagctggacctttggcggcggcaccaaactggaaattaaaccgaaaagctgcgataaaacccatacctgcccgccgtgcccggcgccggaatttcagggcggcccgagcgtgtttctgtttccgccgaaaccgaaagataccctgtatattacccgcgaaccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgccaggtgagcaacaaagcgctgccggcgccgattgaaaaaaccattagcaaagcgaaaggccagccgcgcgaaccgcaggtgtatgtgtatccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagctttgcgctggtgagcaaactgaccgtggataaaagccgctggcagcagggcaacgtgtttagctgcagcgtgatgcatgaagcgctgcataaccattatacccagaaaagcctgagcctgagcccgggc

SEQ ID NO: 16 - DNA, 1419 bp, synthetic construct
  source 1..1419: mol_type = other DNA
gaagtgcagctgcagcagagcggcgcggaactggtgaaaccgggcgcgagcgtgaaactgagctgcaccgcgagcggctttaacattaaagatacctttatgcattgggtgaaactgcgcccggaacagggcctggaatggattggccgcattgatccggcgaacggctataccaaatatgatccgcgctttcagggcaaagcgaccattattgcggataccagcagcaacaccgcgtatctgcagctgagcagcctgaccagcgaagataccgcggtgtattattgcgcgagcggcgaatattatgcgctggattattggggccagggcaccagcgtgaccgtgagcagcggcggcggcggcagcggcggcggcggcagcggcggcggcggcagcgatattgtgctgacccagagcccggcgagcctgaccgtgagcctgggccagcgcgcgaccattagctgccgcgcgagcaaaagcgtgagcaccagcggctatagctatatgcattggtatcagcagaaaccgggccagccgccgaaactgctgatttatctggcgagcaacctggaaagcggcgtgccggcgcgctttagcggcagcggcagcggcaccgattttaccctgaacattcatccggtggaagaagaagatgcggcgacctattattgccagcataaccgcgaactgccgtatacctttggcggcggcaccaaactggaaattaaaccgaaaagctgcgataaaacccatacctgcccgccgtgcccggcgccggaatttcagggcggcccgagcgtgtttctgtttccgccgaaaccgaaagataccctgtatattacccgcgaaccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgccaggtgagcaacaaagcgctgccggcgccgattgaaaaaaccattagcaaagcgaaaggccagccgcgcgaaccgcaggtgtatgtgtatccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagctttgcgctggtgagcaaactgaccgtggataaaagccgctggcagcagggcaacgtgtttagctgcagcgtgatgcatgaagcgctgcataaccattatacccagaaaagcctgagcctgagcccgggc

SEQ ID NO: 17-18 - skipped (2 entries)

SEQ ID NO: 19 - protein, 474 aa, synthetic construct
QVQLVQSGSELKKPGASVKVSCKASGYTFTNFGMNWVRQAPGQGLEWMGWINTYSGEATYADDFKGRFVFSLDTSVSTAYLQISSLKAEDTAVYYCARRGGTAEFDYWGQGTLVTVSSGGGGSGGGGSGGGGSEIVLTQSPDFQSVTPKEKVTITCSASSSISSNFLHWYQQKPDQSPKLLIYRTSKLASGVPSRFSGSGSGTDFTLTINSLEAEDAATYYCQQGIYMPLTFGGGTKLEIKRLEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 20 - DNA, 1429 bp, synthetic construct
  source 1..1429: mol_type = other DNA
ccaggtgcagctggtgcagagcggagcagaggtgaagaagccaggagcctctgtgaaggtgagctgcaaggcctccggctacaccttcacacggtataccatgcactgggtgagacaggcacctggacagggcctggagtggatgggctacatcaacccaagccggggctacacaaactataatcagaagtttaaggacagagtgaccatcacagccgataagagcacctccacagcctatatggagctgagctccctgaggtccgaggacaccgccgtgtactattgcgcccgctactatgacgatcactactgtctggattattggggccagggcaccacagtgacagtgtctagcggcggaggaggctctggaggaggaggcagcggcggcggcggctctggcagcgagatcgtgctgacccagtccccagccacactgtccctgtctccaggagagagggccaccctgagctgctccgcctcctctagcgtgtcttacatgaattggtatcagcagaagcccggcaaggcccctaagaggctgatctacgacacctctaagctggcaagcggagtgccctcccgcttctctggcagcggctccggcaccgactttaccctgacaatcaactccctggaggccgaggatgccgccacatactattgtcagcagtggtcctctaatcctttcacctttggccagggcacaaaggtggagatcaagcggctcgagcccaagagctgcgacaagacccacacctgtcctccatgtcctgctccagagtttcaaggcggcccttccgtgttcctgtttcctccaaagcctaaggacaccctgtacatcacccgcgagcctgaagtgacctgtgtggtggtggatgtgtcccacgaggaccccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgccaggtgtccaacaaggccctgcctgctcctatcgagaaaaccatcagcaaggccaagggccagcctagggaacctcaagtgtacgtgtaccctcctagccgggacgagctgaccaagaatcaggtgtccctgacctgcctcgtgaagggcttctacccttccgacatcgccgtggaatgggagagcaatggccagcctgagaacaactacaagacaacccctcctgtgctggacagcgacggctcttttgccctggtgtccaagctgacagtggacaagtccagatggcagcagggcaacgtgttcagctgcagcgtgatgcacgaggccctgcacaaccactacacccagaagtccctgagcctgtctcctggatga

SEQ ID NO: 21 - skipped

SEQ ID NO: 22 - protein, 726 aa, synthetic construct
EVQLQQSGPELEKPGASVKISCKASGYSFTGYNMNWVKQSNGKSLEWIGNIDPYYGGSDYSQKFLGKATLTVDKSSSTAYMHLKSLTSEDSAVYYCARSNMYDGEYYVMDYWGQGTSVTVSSGGGGSGGGGSGGGGSDIVLTQSPASLAVSLGQRATISCRASESVDNFGVSFMNWFQQKPGQPPKLLIYASSNRGSGVPARFSGSGSGTDFSLNVHPMEEDDAAMYFCQQSKAVPRTFGGGTKLEIKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGEVQLQQSGPELEKPGASVKISCKASGYSFTGYNMNWVKQSNGKSLEWIGNIDPYYGGSDYSQKFLGKATLTVDKSSSTAYMHLKSLTSEDSAVYYCARSNMYDGEYYVMDYWGQGTSVTVSSGGGGSGGGGSGGGGSDIVLTQSPASLAVSLGQRATISCRASESVDNFGVSFMNWFQQKPGQPPKLLIYASSNRGSGVPARFSGSGSGTDFSLNVHPMEEDDAAMYFCQQSKAVPRTFGGGTKLEIK

SEQ ID NO: 23 - protein, 722 aa, synthetic construct
DVQLVESGGGLVQPGGSRKLSCAASGFTFSNFGMHWVRQAPEKGLEWVAYVSRGSTTFYYADTVKGRFTISRDNPKNTLFLQMTSLRSEDTAMYYCARSATATWTMDYWGHGTSVTVSSGGGGSGGGGSGGGGSNIMMTQSPSSLAVSAGEKVTMSCKSSQSVFSSSNHMNYLAWYQQKPGQSPKLLIYWASTRESGVPDRFTGSGSGTDFTLTISSVQAEDLAVYYCHQYLSSWTFGGGSKLEIKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGDVQLVESGGGLVQPGGSRKLSCAASGFTFSNFGMHWVRQAPEKGLEWVAYVSRGSTTFYYADTVKGRFTISRDNPKNTLFLQMTSLRSEDTAMYYCARSATATWTMDYWGHGTSVTVSSGGGGSGGGGSGGGGSNIMMTQSPSSLAVSAGEKVTMSCKSSQSVFSSSNHMNYLAWYQQKPGQSPKLLIYWASTRESGVPDRFTGSGSGTDFTLTISSVQAEDLAVYYCHQYLSSWTFGGGSKLEIK

SEQ ID NO: 24 - protein, 712 aa, synthetic construct
QVQLQQSAPELARPGASVRMSCKASGYTFTTYTMNWLTQRPGQGLEWIGYINPSSGYTEYNQKFKDKTSLTADTSSSTAYMQLSSLTSEHSAVYYCARASGYSSWFAYWGQGTLVTVSAGGGGSGGGGSGGGGSNIVMTQTPKFLLVSAGDRVTITCKASQSVNNDVGWYQQKPGQSPKLLIYYASNRYTGVPDRFTGSGYGTDFTFTISTVQAEDLAVYFCQQDYNSPLTFGAGTKLELKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGQVQLQQSAPELARPGASVRMSCKASGYTFTTYTMNWLTQRPGQGLEWIGYINPSSGYTEYNQKFKDKTSLTADTSSSTAYMQLSSLTSEHSAVYYCARASGYSSWFAYWGQGTLVTVSAGGGGSGGGGSGGGGSNIVMTQTPKFLLVSAGDRVTITCKASQSVNNDVGWYQQKPGQSPKLLIYYASNRYTGVPDRFTGSGYGTDFTFTISTVQAEDLAVYFCQQDYNSPLTFGAGTKLELK

SEQ ID NO: 25 - protein, 704 aa, synthetic construct
QVQLQQSGAELARPGASVKMSCKASGYTFTFYAILWVKQMPGQGLEWIGFINPSSGYTSYNQKFKDKATLTADKSSSTAYMQLSSLTSEDSAVYYCARDYGLDYWGQGTTLAVSSGGGGSGGGGSGGGGSDIVMTQAAPSVPVTPGESVSISCRSSKSLLHSNGNTYLYWFLQRPGQSPQLLIYRMSNLASGVPDRFSGSGSGTAFTLGISRVEAGDVGVYYCMQHLEYPFTFGSGTKLEIKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGQVQLQQSGAELARPGASVKMSCKASGYTFTFYAILWVKQMPGQGLEWIGFINPSSGYTSYNQKFKDKATLTADKSSSTAYMQLSSLTSEDSAVYYCARDYGLDYWGQGTTLAVSSGGGGSGGGGSGGGGSDIVMTQAAPSVPVTPGESVSISCRSSKSLLHSNGNTYLYWFLQRPGQSPQLLIYRMSNLASGVPDRFSGSGSGTAFTLGISRVEAGDVGVYYCMQHLEYPFT

SEQ ID NO: 26 - protein, 722 aa, synthetic construct
DVQLVESGGGLVQPGGSRKLSCAASGFTFSSFGMHWVRQAPEKGLEWVAYINSGSTTFYYADTVKGRFTISRDNPKNTLFLQMTSLGSEDTAMYYCARSATATWTMDYWGQGTSVTVSSGGGGSGGGGSGGGGSNIMMTQSPSSLAVSAGEKVTMSCKSSQSVFVSSNQKNYLAWYQQKPGQSPKLLIYWASTRESAVPDRFTGSGSGTDFTLTIGSVQAEDLAVYYCHQYLSSWTFGGGTKLEIKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGDVQLVESGGGLVQPGGSRKLSCAASGFTFSSFGMHWVRQAPEKGLEWVAYINSGSTTFYYADTVKGRFTISRDNPKNTLFLQMTSLGSEDTAMYYCARSATATWTMDYWGQGTSVTVSSGGGGSGGGGSGGGGSNIMMTQSPSSLAVSAGEKVTMSCKSSQSVFVSSNQKNYLAWYQQKPGQSPKLLIYWASTRESAVPDRFTGSGSGTDFTLTIGSVQAEDLAVYYCHQYLSSWTFGGGTKLEIK

SEQ ID NO: 27 - protein, 716 aa, synthetic construct
EVQLQQSGAELVKPGASVKLSCTASGFNIKDTFMHWVKLRPEQGLEWIGRIDPANGYTKYDPRFQGKATIIADTSSNTAYLQLSSLTSEDTAVYYCASGEYYALDYWGQGTSVTVSSGGGGSGGGGSGGGGSDIVLTQSPASLTVSLGQRATISCRASKSVSTSGYSYMHWYQQKPGQPPKLLIYLASNLESGVPARFSGSGSGTDFTLNIHPVEEEDAATYYCQHNRELPYTFGGGTKLEIKPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVYPPSRDELTKNQVSLTCLVKGFYPSDIAVEWESNGQPENNYKTTPPVLDSDGSFALVSKLTVDKSRWQQGNVFSCSVMHEALHNHYTQKSLSLSPGEVQLQQSGAELVKPGASVKLSCTASGFNIKDTFMHWVKLRPEQGLEWIGRIDPANGYTKYDPRFQGKATIIADTSSNTAYLQLSSLTSEDTAVYYCASGEYYALDYWGQGTSVTVSSGGGGSGGGGSGGGGSDIVLTQSPASLTVSLGQRATISCRASKSVSTSGYSYMHWYQQKPGQPPKLLIYLASNLESGVPARFSGSGSGTDFTLNIHPVEEEDAATYYCQHNRELPYTFGGGTKLEIK

SEQ ID NO: 28 - skipped

SEQ ID NO: 29 - protein, 15 aa, synthetic construct
GGGGSGGGGSGGGGS

SEQ ID NO: 30 - DNA, 45 bp, synthetic construct
  source 1..45: mol_type = other DNA
ggaggcggaggttctggcggcggaggaagtggtggcggaggctct

SEQ ID NO: 31 - protein, 612 aa, synthetic construct
QVQLVQSGSELKKPGASVKVSCKASGYTFTNFGMNWVRQAPGQGLEWMGWINTYSGEATYADDFKGRFVFSLDTSVSTAYLQISSLKAEDTAVYYCARRGGTAEFDYWGQGTLVTVSSGGGGSGGGGSGGGGSEIVLTQSPDFQSVTPKEKVTITCSASSSISSNFLHWYQQKPDQSPKLLIYRTSKLASGVPSRFSGSGSGTDFTLTINSLEAEDAATYYCQQGIYMPLTFGGGTKLEIKRLEPKSCDKTHTCPPCPAPEFQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYVLPPSRDELTKNQVSLLCLVKGFYPSDIAVEWESNGQPENNYLTWPPVLDSDGSFFLYSKLTVDKSRWQQGNVFSCSVMHEALHNHYTQKSLSLSPGQVQLVQSGSELKKPGASVKVSCKASGYTFTNFGMNWVRQAPGQGLEWMGWINTYSGEATYADDFKGRFVFSLDTSVSTAYLQISSLKAEDTAVYYCARRGGTAEFDYWGQGTLVTVSSGGGGSGGGGSGGGGSEIVLT